(19) United States
(12) Patent Application Publication
(10) Pub. No.: US 2024/0101988 A1
(43) Pub. Date: Mar. 28, 2024

(54) TEMPERATURE-SENSITIVE CAS9 PROTEIN

(71) Applicant: Novozymes A/S, Bagsvaerd (DK)

(72) Inventors: Carsten Andersen, Vaerloese (DK); Poul Erik Pedersen, Farum (DK); Michael Dolberg Rasmussen, Vallensbaek (DK); Carsten Hjort, Smoerum (DK)

(73) Assignee: Novozymes A/S, Bagsvaerd (DK)

(21) Appl. No.: 18/355,170

(22) Filed: Jul. 19, 2023

Related U.S. Application Data
(63) Continuation of application No. 16/652,920, filed on Apr. 1, 2020, now abandoned.

(30) Foreign Application Priority Data
Nov. 10, 2017 (EP) 17201135.5

(51) Int. Cl. C12N 9/22; C12N 15/63

(52) U.S. Cl. CPC C12N 9/22; C12N 15/63; C07K 2319/35; C12Y 302/01001

(57) ABSTRACT

The present invention relates to temperature-sensitive variants of a Class-II Cas9 protein, polynucleotides encoding said variants, nucleic acid constructs and expression vectors comprising polynucleotides encoding said variants, host cells expressing said variant, methods of transient repression and expression of one or more genome target sequence using said variants, and use of said variants, polynucleotides, nucleic acid constructs, expression vectors, host cells, and methods.

### CROSS-REFERENCE TO RELATED APPLICATIONS

This application is a continuation of pending U.S. patent application Ser. No. 16/652,920 which is a 35 U.S.C. 371 national application of PCT/EP2018/080508, filed Nov. 7, 2018, which claims priority or the benefit from European Patent Application No. 17201135.5, filed Nov. 10, 2017. The contents of these applications are fully incorporated herein by reference.

### REFERENCE TO SEQUENCE LISTING

This application contains a Sequence Listing in computer-readable form. The contents of the electronic sequence listing created on Dec. 1, 2023, named SQ_ST26.xml and 53 KB in size, is hereby incorporated by reference in its entirety.

### FIELD OF THE INVENTION

The present invention relates to temperature-sensitive variants of a Class-II Cas9 protein, polynucleotides encoding said variants, nucleic acid constructs and expression vectors comprising polynucleotides encoding said variants, host cells expressing said variant, methods of expressing and repressing one or more genome target sequence using said variants, and use of said variants, polynucleotides, nucleic acid constructs, expression vectors, host cells, and methods.

### BACKGROUND OF THE INVENTION

The so-called CRISPR-Cas9 genome editing system has been widely used as a tool to modify the genomes of a number of organisms. The power of the CRISPR-Cas9 system lies in its simplicity to target and edit down to a single base pair in a specific gene of interest. The Cas9 protein is a dual-RNA guided endonuclease, with the nuclease activity being directed by so-called guide-RNA (gRNA) molecules. The choice of Cas9 target sequence is made by changing a 20 bp sequence window of the gRNA to match the target DNA sequence. When complexed with the gRNA molecule, the Cas9 protein will then bind its target DNA sequence and create a double-stranded break using two catalytic domains. Cas9 may be further engineered to contain a single amino acid mutation in either one of the two catalytic domains. In this case, Cas9 functions as a nickase, i.e. with single-strand cleavage activity.

In addition to its use within genome editing, the CRISPR-Cas9 system has also been used for control of gene expression. This application, often referred to as CRISPR inhibition or CRISPRi, allow sequence-specific repression or activation of a gene. CRISPR interference utilizes a catalytically inactive (dead) Cas9 variant (termed Cas9d) lacking endonuclease activity. The Cas9d-gRNA complex retains the ability to bind to the target DNA sequence, but cannot introduce any breaks in the DNA strand. By varying the gRNA sequence, one can control the target DNA sequence for the Cas9d-gRNA complex and thereby regulate the expression of virtually any gene in any organism. However, since the complex formed between the Cas9d-gRNA complex and the target DNA (the Cas9d-gRNA-DNA complex) is very stable and difficult to reverse, CRISPR inhibition remains unsuitable for transient control of gene expression.

The homodimeric Rhodobacter sphaeroides light-oxygen-voltage domain polypeptide (RsLOV) dissociates into its two polypeptide monomers, each consisting of 176 amino acids, when exposed to blue light. RsLOV can confer light sensitivity onto other polypeptides when fused with or inserted into them. Some RsLOV-Cas9/Cas9d hybrid constructs have been shown to be both light- and temperature sensitive (Richter F. et al. 2016, Engineering of temperature- and light-switchable Cas9 variants, Nucleic Acids Research, 2016, Vol. 44, No. 20, p. 10003-10014).

WO 2013/176772 suggests fusion proteins consisting of Cas9 and various heterologous polypeptides. The heterologous polypeptide may confer additional properties to the Cas9 fusion protein.

### SUMMARY OF THE INVENTION

The present invention provides temperature-sensitive variants of a Class-II Cas9 protein, polynucleotides encoding said variants, nucleic acid constructs and expression vectors comprising polynucleotides encoding said variants, host cells expressing said variant, methods of expressing and repressing one or more genome target sequence using said variants, and use of said variants, polynucleotides, nucleic acid constructs, expression vectors, host cells, and methods.

The invention is based on the surprising finding that introduction of specific amino acid alterations in the S. pyogenes Cas9 protein results in temperature-sensitive Cas9 variants (tsCas9). When employing these tsCas9 variants in vivo together with a suitable guide-RNA and a target genomic sequence, the initial formation of the tsCas9-gRNA complex as well as the formed tsCas9-gRNA-DNA become temperature-sensitive. Thus, the gRNA and DNA binding properties of the tsCas9 variant can be controlled by shifting the temperature up and/or down, depending on what is desired.

Thus, in a first aspect, the present invention relates to a temperature-sensitive variant of a Class-II Cas9 protein (tsCas9), said variant comprising at least one alteration of one or more amino acid important for protein stability or for stability of a complex formed between the Class-II Cas9 protein, one or more guide-RNA (gRNA), and one or more corresponding genome target sequence, wherein the at least one alteration is a substitution, insertion, or deletion of 1-10 amino acids; preferably the at least one alteration is a substitution or deletion of 1-10 amino acids; most preferably the at least one alteration is a substitution.

In a second aspect, the present invention relates to polynucleotides encoding a tsCas9 of the first aspect.

In a third aspect, the present invention relates to nucleic acid constructs comprising a polynucleotide of the second aspect.

In a fourth aspect, the present invention relates to a nucleic acid construct comprising a polynucleotide of the second aspect.

In a fifth aspect, the present invention relates to a host cell comprising a tsCas9 as defined in the first aspect, a polynucleotide as defined in the second aspect, a nucleic acid construct as defined in the third aspect, and/or an expression vector as defined in the fourth aspect.

In a sixth aspect, the present invention relates to a method of inducing expression of one or more genome target sequence of interest, the method comprising the steps of:
a) providing a host cell according to the fifth aspect, said host cell further comprising one or more suitable gRNA and one or more genome target sequence of interest;
b) cultivating the host cell at a dissociative temperature of the tsCas9, whereby a complex formed in the host cell by the tsCas9 with the one or more suitable gRNA and the one or more genome target sequence of interest disassociates; and subsequently
c) lowering the temperature to a restrictive temperature of the tsCas9 and cultivating the host cell, whereby expression of the one or more target sequence is induced.

In a seventh aspect, the present invention relates to a method of repressing one or more genome target sequence of interest, the method comprising the steps of:
a) providing a host cell according to the fifth aspect, said host cell further comprising one or more suitable gRNA and one or more genome target sequence of interest;
b) cultivating the host cell at a restrictive temperature of the tsCas9, wherein the one or more genome target sequence of interest is expressed; and subsequently
c) lowering the temperature to a permissive temperature of the tsCas9, whereby a complex is formed in the host cell by the tsCas9, the one or more suitable gRNA and the one or more genome target sequence of interest, and whereby expression of the one or more genome target sequence is repressed.

The present invention also relates to use of a tsCas9 according to the first aspect, a polynucleotide according to the second aspect, a nucleic acid construct according to the third aspect, an expression vector according to the fourth aspect, a host cell according to the fifth aspect, and/or a method according to the sixth and seventh aspects.

### BRIEF DESCRIPTION OF DRAWINGS

FIG. 1 shows a schematic view of the two anti-parallel amyL gene copies inserted into the B. licheniformis SJ4671 strain mentioned in Example 1. The copies are spaced by approximately 2.5 Kb originating from non-functional DNA of the B. subtilis chromosome (SEQ ID NO:3).

FIG. 2 shows a schematic view of an additional copy of the amyL gene inserted at the xyl locus (SEQ ID NO: 4) in the SJ4671 strain in Example 1.

FIG. 3 shows a schematic view of an additional copy of the amyL gene inserted at the gnt locus (SEQ ID NO: 5) in Example 1 to form strain SJ6026.

FIG. 4 shows a schematic view of the prsA gene inserted at the mprL locus (SEQ ID NO: 6) in Example 1 to form strain MOL2173.

FIG. 5 shows a schematic view of an expression cassette consisting of the cas9d gene integrated at the forD locus and expressed from the forD promoter, the gDNA(P4199) transcribed from the PamyQsc promoter and the cat gene conferring chloramphenicol resistance (SEQ ID NO:7) in Example 2.

FIGS. 6a and 6b show illustrations of the two B. licheniformis strains PP5007 and PP5021, respectively.

FIG. 7 shows alpha-amylase expression using the PP5007 and PP5021 strains in Example 3 with and without repression.

FIG. 8 shows a schematic view of a DNA fragment inserted in Example 4 at the amyE locus where the GFP gene encoding the green fluorescent protein is expressed from the amyL variant promoter P4199. Furthermore a gDNA(P4199) is expressed from the PamyQ consensus promoter (PamyQsc) The final map of the amyE locus after integration is shown.

FIG. 9 shows a schematic view of an expression cassette inserted in Example 5 at the pel locus with the cas9d gene encoding the Cas9d protein.

FIG. 10 shows a schematic view of the strain PP5336 constructed in Example 5.

FIG. 11 shows an example of the testing of the different variant clones for GFP fluorescence in Example 8 at the different temperatures.

FIGS. 12a, 12B, 12c, 12d, 12e and 12f show the testing of the Cas9d variants in Example 9 in liquid cultures at different temperatures.

FIGS. 13a and 13b shows the testing of the Cas9d variants in Example 9 in liquid cultures after temperature shifts.

FIG. 14 shows a schematic view of an illustration of the CRISPRi complex binding to the promoter region of promoter P4199 to repress the expression of the gene operably linked with the P4199 promoter.

FIG. 15 shows a fed-batch experiment with B. licheniformis strains containing temperature sensitive variants of Cas9d, gDNA(P4199), and four copies of an amylase expressed from the P4199 promoter. The cultures were after inoculation incubated for two days at either 30° C. or 42° C. The temperature was then shifted to 42° C. and 30° C., respectively, and cultures continued growth for additional three days. Samples were taken for measurement of amylase activities.

FIG. 16 shows a fed-batch experiment with B. licheniformis strains containing temperature sensitive variants of Cas9d, gDNA(P4199), and four copies of an amylase expressed from the P4199 promoter. The cultures were after inoculation incubated for two days at either 30° C. or 42° C. The temperature was then shifted from 42° C. to 35° C., 37° C., and 39° C., or from 30° C. to 42° C. Culture TM(P)093 was also spiked with a shift to 48° C. for a short period. The cultures continued growth for additional three days. Samples were taken for measurement of amylase activities.

FIG. 17 shows measurement of GFP after incubation of B. subtilis strains containing temperature-sensitive variants of Cas9d and gDNA(gfp). Two replicates of each strains were cultivated. The data show a trend with increased temperature sensitivity of the constructed variants, which is in good agreement to what was observed on plates.

FIG. 18 shows a fed-batch experiment with B. licheniformis strains containing temperature sensitive variants of Cas9d, gDNA(P4199), and four copies of an amylase expressed from the P4199 promoter. The cultures were after inoculation incubated for two days at either 30° C. or 44° C. The temperature was then shifted to 44° C. and 30° C., respectively, and cultures continued growth for additional three days. Samples were taken for measurement of amylase activities.

### DEFINITIONS

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Temperature-sensitive variant of a Class-II Cas9 protein: The terms “temperature-sensitive variant of a Class-II Cas9 protein” and “tsCas9” are used interchangeably herein.

Permissive temperature: The term “permissive temperature” means a temperature or temperature range, where the tsCas9 behaves as its wildtype parent. The permissive temperature is the temperature or temperature range where the tsCas9 is able to form a complex with one or more gRNA and the corresponding one or more genome target sequence of interest and either cut the target sequence in the case of Cas9, nick the target sequence in the case of Cas9 nickase, or stay bound to the target sequence in the case of the nuclease-null Cas9 variant termed Cas9d. The permissive temperature range is defined mainly by the choice of host cell and the specific temperature-sensitive variant of a Class-II Cas9 protein as well as gRNA(s) applied.

Dissociative temperature: The term “dissociative temperature” means a temperature or temperature range, where a complex formed between the tsCas9 of the instant invention, one or more gRNA, and the corresponding one or more genome target sequence of interest dissociates.

Restrictive temperature: The term “restrictive temperature” means a temperature or temperature range, where the temperature-sensitive variant of Class-II Cas9 protein of the instant invention is unable to form a complex with one or more gRNA and the corresponding one or more genome target sequence of interest.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity” or “sequence complementarity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity (or corresponding sequence complementarity) between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

To determine the % complementarity of two complementary sequences, one of the two sequences needs to be converted to its complementary sequence before the % complementarity can then be calculated as the % identity between the first sequence and the second converted sequences using the above-mentioned algorithm.

Stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

Variant: The present invention relates to variants of the mature polypeptide of SEQ ID NO: 2 comprising at least one amino acid alteration, i.e., at least one substitution, deletion, and/or insertion at one or more (e.g., several) positions. In an embodiment, the number of alterations introduced into the mature polypeptide of SEQ ID NO: 2 is up to 10, e.g., 1, 2, 3, 4, 5, 6, 7, 8, 9, or 10. Alterations include conservative substitutions, wherein an amino acid is substituted with another amino acid with similar physicochemical properties; non-conservative substitutions, wherein an amino acid is substituted with another amino acid with different physicochemical properties; small insertions, typically of typically of 1-30 amino acids; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Common substitutions include, but are not limited to, Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In the context of temperature-sensitive protein variants, a conservative substitution may also be defined a substitution that has no detrimental effect on protein stability at the permissive temperature of the variant.

### Conventions for Designation of Variants

For purposes of the present invention, the mature polypeptide disclosed in SEQ ID NO: 2 is used to determine the corresponding amino acid residue in another Class-II Cas9 protein. The amino acid sequence of another Class-II Cas9 protein is aligned with the mature polypeptide disclosed in SEQ ID NO: 2, and based on the alignment, the amino acid position number corresponding to any amino acid residue in the mature polypeptide disclosed in SEQ ID NO: 2 may be determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another Class-II Cas9 protein can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other protein has diverged from the mature polypeptide of SEQ ID NO: 2 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions: For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411 Phe” or “G205R+S411F”, representing substitutions at positions 205 and 411 of glycine (G) with arginine (R) and serine (S) with phenylalanine (F), respectively.

Deletions: For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions: For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G-K-A |

Multiple alterations: Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations: Where different alterations can be introduced at a position, the different alterations are separated by a comma, e.g., “Arg170Tyr,Glu” and R170Y,E represents a substitution of arginine at position 170 with tyrosine or glutamic acid. Thus, “Tyr167Gly,Ala+Arg170Gly,Ala” designates the following variants:
“Tyr167Gly+Arg170Gly”,
“Tyr167Gly+Arg170Ala”,
“Tyr167Ala+Arg170Gly”, and
“Tyr167Ala+Arg170Ala”.

### DETAILED DESCRIPTION OF THE INVENTION

The present invention provides means and methods for utilizing the versatility and precision of the CRISPR-Cas9 system for expression and repression of expression of one or more genome target sequence of interest. The invention is based on the surprising finding that introduction of specific amino acid substitutions in the Cas9 protein results in destabilized, temperature-sensitive Cas9 variants. When employing these temperature-sensitive Cas9 variants in genome editing or CRISPR inhibition, the stability of the initial Cas9-gRNA complex as well as the Cas9-gRNA-DNA complex also becomes destabilized and temperature-sensitive. Thus, by applying temperature-sensitive Cas9 variants in CRISPR inhibition, the gRNA and DNA binding properties of the Cas9 variant may be controlled by shifting the temperature.

Thus, in one aspect, the present invention relates to a temperature-sensitive variant of a Class-II Cas9 protein (tsCas9), said variant comprising at least one alteration of one or more amino acid important for protein stability or for stability of a complex formed between the Class-II Cas9 protein, one or more guide-RNA (gRNA), and one or more corresponding genome target sequence, wherein the at least one alteration is a substitution, insertion, or deletion of 1-10 amino acids; preferably the at least one alteration is a substitution or deletion of 1-10 amino acids; most preferably the at least one alteration is a substitution.

### Temperature-Sensitive Variants

The present invention provides temperature-sensitive variants of a Class-II Cas9 protein comprising at least one amino acid alteration (e.g., at least one substitution, insertion, and/or deletion) in a position important for Class-II Cas9 protein stability or for stability of a complex formed between a Class-II Cas9 protein, one or more gRNA, and one or more genome target sequence. Temperature-sensitive variants of a Class-II Cas9 protein may be obtained from any parent Class-II Cas9 protein. Preferably, temperature-sensitive variants are obtained from S. pyogenes Cas9 (SEQ ID NO:2).

The temperature-sensitive variants of a Class-II Cas9 protein may be a nickase or nuclease-null variant. Preferably, such variants comprise an amino acid alteration in a position corresponding to position 10 and/or position 840 of SEQ ID NO:2. More preferably, such variants comprise a substitution of aspartic acid for alanine, D10A, and/or a substitution of histidine for alanine, H840A.

Preferably, the at least one amino acid alteration occurs in a position within the sequence of the parent Class-II Cas9 protein, i.e., the at least one amino acid alteration is not an N- or C-terminal extension, insertion, or fusion.

The at least one alteration in a position important for Class-II Cas9 protein stability or for stability of a complex formed between a Class-II Cas9 protein, one or more gRNA, and one or more genome target sequence may occur in a position selected from the group consisting of T13, N14, S29, K31, F32, L35, K44, N46, S55, E57, T62, R63, K65, R66, R69, R70, R71, Y72, R74, R75, R78, S104, F105, V107, E108, R115, H116, V126, H129, Y136, H160, K163, F164, R165, P176, S179, E223, N235, E260, D261, D269, T310, P316, Y325, H328, H329, K336, R340, Y347, F351, F352, I363, D364, R403, P411, Q413, I414, Y430, F432, I448, Y450, P454, L455, R457, N459, S460, R461, F462, R467, T472, F491, M495 N497, Y515, R557, G582, R653, T657, R661, Q695, H698, S719, L720, H721, K735, L738, K742, M751, R765 S777, E779, D850, Q894, E910, L911, F916, K918, Q920, T924, R925, Q926, K929, Q933, R951, S960, S964, K968, R976, V982, Y1013, G1030, A1032, K1085, M1089, P1090, Q1091, T1098, E1099, V1100, T1102, G1103, F1105, K1107 E1108, S1109, K1113, R11414, N1115, S1116, D1117, K1118, K1123, K1124, F1134, D1135, W1126, K1135, R1171, K1197, K1211, S1216, E1219, E1243, R1279, D1284, P1301, H1311, R1333, K1334, R1335, T1337, S1338, H1349, Y1356, and T1358 of SEQ ID NO:2.

Preferably, the at least one alteration occur in a position important for Class-II Cas9 protein-protein interactions including, but not limited to, a position corresponding to P176, S179, E223, E260, D261, T310, P316, P411, Q413, I414, Y430, F432, R457, N459, R461, R557, R653, E910, L911, R951, K1123, W1126, P1301, and H1311 of SEQ ID NO:2.

Also preferably, the at least one alteration occur in a position important for interactions between a Class-II Cas9 protein and one or more gRNA including, but not limited to, a position corresponding to S29, K31, F32, L35, K44, N46, E57, T62, R63, K65, R66, R69, R70, R71, Y72, R74, R75, R78, S104, F105, V107, E108, R115, H116, V126, H129, Y136, H160, K163, F164, R165, Y325, H328, H329, K336, R340, Y347, F351, F352, I363, D364, R403, I448, P454, L455, R457, N459, S460, R461, F462, R467, T472, Y515, R661, S719, L720, H721, K735, L738, K742, M751, S777, E779, D850, K918, Q933, V982, K1085, M1089, P1090, Q1091, T1098, E1099, V1100, T1102, G1103, F1105, E1108, K1113, K1123, K1124, F1134, R1171, K1197, K1211, R1279, H1349, Y1356, and T1358 of SEQ ID NO:2.

Also preferably, the at least one alteration occur in a position important for interactions between a Class-II Cas9 protein and one or more genome target sequence including, but not limited to, a position corresponding to T13, N14, S55, D269, Y450, F491, M495, N497, G582, T657, R661, Q695, H698, R765, S777, Q894, F916, K918, Q920, T924, R925, Q926, K929, S960, S964, K968, R976, Y1013, G1030, A1032, K1107, E1108, S1109, R1114, N1115, S1116, D1117, K1118, D1135, S1216, E1219, E1243, D1284, R1333, K1334, R1335, T1337, and S1338 of SEQ ID NO:2.

Also preferably, the at least one alteration occur in a position important for Class-II Cas9 protein-protein interactions and/or for interactions between a Class-II Cas9 protein and one or more gRNA including, but not limited to, a position corresponding to S29, K31, F32, L35, K44, N46, E57, T62, R63, K65, R66, R69, R70, R71, Y72, R74, R75, R78, S104, F105, V107, E108, R115, H116, V126, H129, Y136, H160, K163, F164, R165, P176, S179, E223, E260, D261, T310, P316, Y325, H328, H329, K336, R340, Y347, F351, F352, I363, D364, R403, P411, Q413, I414, Y430, F432, I448, P454, L455, R457, N459, S460, R461, F462, R467, T472, Y515, R557, R653, R661, S719, L720, H721, K735, L738, K742, M751, S777, E779, D850, E910, L911, K918, Q933, R951, V982, K1085, M1089, P1090, Q1091, T1098, E1099, V1100, T1102, G1103, F1105, E1108, K1113, K1123, K1124, F1134, W1126, R1171, K1197, K1211, R1279, P1301, H1311, H1349, Y1356, and T1358 of SEQ ID NO:2.

Also preferably, the at least one alteration occur in a position important for Class-II Cas9 protein-protein interactions and/or for interactions between a Class-II Cas9 protein and one or more genome target sequence including, but not limited to, a position corresponding to T13, N14, S55, P176, S179, E223, E260, D261, D269, T310, P316, P411, Q413, I414, Y430, F432, Y450, R457, N459, R461, F491, M495, N497, R557, G582, R653, T657, R661, Q695, H698, R765, S777, Q894, E910, L911, F916, K918, Q920, T924, R925, Q926, K929, R951, S960, S964, K968, R976, Y1013, G1030, A1032, K1107, E1108, S1109, R1114, N1115, S1116, D1117, K1118, K1123, W1126, D1135, S1216, E1219, E1243, D1284, P1301, H1311, R1333, K1334, R1335, T1337, and S1338 of SEQ ID NO:2.

Also preferably, the at least one alteration occur in a position important for interactions between a Class-II Cas9 protein and one or more gRNA and/or for interactions between a Class-II Cas9 protein and one or more genome target sequence including, but not limited to, a position corresponding to T13, N14, S29, K31, F32, L35, K44, N46, S55, E57, T62, R63, K65, R66, R69, R70, R71, Y72, R74, R75, R78, S104, F105, V107, E108, R115, H116, V126, H129, Y136, H160, K163, F164, R165, D269, Y325, H328, H329, K336, R340, Y347, F351, F352, I363, D364, R403, I448, Y450, P454, L455, R457, N459, S460, R461, F462, R467, T472, F491, M495, N497, Y515, G582, T657, R661, Q695, H698, S719, L720, H721, K735, L738, K742, M751, R765, S777, E779, D850, Q894, F916, K918, Q920, T924, R925, Q926, K929, Q933, S960, S964, K968, R976, V982, Y1013, G1030, A1032, K1085, M1089, P1090, Q1091, T1098, E1099, V1100, T1102, G1103, F1105, K1107, E1108, S1109, K1113, R1114, N1115, S1116, D1117, K1118, K1123, K1124, F1134, D1135, R1171, K1197, K1211, S1216, E1219, E1243, R1279, D1284, R1333, K1334, R1335, T1337, S1338, H1349, Y1356, and T1358 of SEQ ID NO:2.

More preferably, the at least one amino acid alteration occur in a position corresponding to a position selected from the group consisting of S104, F105, V107, P176, S179, E223, N235, E260, D261, T310, P316, P411, Q413, I414, Y430, F432, R457, N459, P475, W476, R557, R653, Q739, E910, L911, R951, K1123, W1126S, and H1311 of SEQ ID NO:2; most preferably the at least one alteration is in a position corresponding to a position selected from the group consisting of S104, F105, V107, P176, S179, E223, E260, D261, T310, P316, P411, Q413, I414, Y430, F432, R457, N459, P475, W476, R557, R653, Q739, E910, L911, R951, K1123, W1126S, and H1311 of SEQ ID NO:2.

The at least one amino acid alteration occurring in a position important for Class-II Cas9 protein stability or for stability of a complex formed between a Class-II Cas9 protein, one or more gRNA, and one or more genome target sequence may be selected from the group consisting of T13S, T13A, N14D, N14S, N14A, S29A, S29G, S29T, S29V, K31R, K31Q, K31H, K31M, K31L, F32Y, F32H, F32H, F32L, F32I, F32M, L35V, L35A, L35T, L35I, L35M, L35F, L35H, K44R, K44Q, K44H, K44M, K44L, K44D, K44N, K44H, K44M, K44L, N46D, N46S, N46T, N46Q, S55A, E57Q, E57D, E57N, E57H, T62S, T62A, T62V, T62P, R63K, R63H, R63Q, R63E, K65R, K65H, K65Q, K65M, K65L, R66K, R66H, R66Q, R66E, R69K, R69H, R69Q, R69E, R70K, R70H, R70Q, R70E, R71K, R71H, R71Q, R71E, Y72F, Y72H, Y72L, Y72M, Y72I, Y72W, R74K, R74H, R74Q, R74E, R75K, R75H, R75Q, R75E, R78K, R78H, R78Q, R78E, S104A, S104A, S104T, S104V, F105H, F105L, F105M, F105I, F105Y, F105W, V107S, V107A, V107I, V107L, V107T, E108Q, E108D, E108N, R115K, R115H, R115Q, R115E, H116F, H116Y, H116Q, H116E, H116N, H116D, H116S, H116T, V126I, V126L, V126A, V126T, V126S, H129F, H129Y, H129Q, H129E, H129N, H129D, H129S, H129T, Y136F, Y136H, Y136L, Y136M, Y136I, Y136W, H160F, H160Y, H160Q, H160E, H160N, H160D, H160S, H160T, K163R, K163H, K163Q, K163E, K163M, K163L, F164Y, F164H, F164M, F164L, F164I, F164W, R165K, R165H, R165Q, R165E, P176A, P176G, P176S, P176T, P176V, S179A, S179G, S179T, S179V, S179P, E223Q, E223D, E223N, E223H, E223S, E223A, E223I, E223L, E223T, E223V, E223P, E223K, E223Y, E223W, E223F, E223G, E223C, E223R, N235NGSGAGGSY, E260Q, E260S, D261N, D261S, D269N, D269S, D269A, T310A, T310G, P316G, P316D, Y325F, Y325H, Y325L, Y325M, Y325I, Y325W, H328F, H328Y, H328Q, H328E, H328N, H328D, H328S, H328T, H329F, H329Y, H329Q, H329E, H329N, H329D, H329S, H329T, K336R, K336H, K336M, K336L, R340K, R340H, R340Q, R340E, Y347F, Y347H, Y347M, Y347L, Y347I, F351H, F351Y, F351M, F351L, F351I, F351W, F352H, F352Y, F352M, F352L, F352I, F352W, I363F, I363L, I363M, I363V, I363A, I363T, D364E, D364Q, D364H, D364N, D364S, D364T, R403K, R403H, R403Q, R403E, P411A, P411G, Q413N, Q413A, I414V, I414A, Y430F, Y430V, F432H, F432V, I448L, I448M, I448V, I448A, Y450H, Y450L, Y450F, P454A, P454G, P454S, P454T, P454V, L455I, L455V, L455M, L455T, L455N, L455F, L455A, R457K, R457H, R457Q, R457E, R457S, N459D, N459E, N459Q, N459H, N459S, N459T, N459A, S460A, S460G, S460T, S460V, R461K, R461H, R461Q, R461E, R461S, F462Y, F462H, F462L, F462I, F462V, F462W, R467K, R467H, R467Q, R467E, T472A, T472P, T472S, T472V, F491H, F491L, M495K, M495Q, M495L, N497D, N497Q, N497E, N497S, Y515F, Y515H, Y515M, Y515L, R557K, R557S, G582A, G582S, G582T, G582V, R653H, R653N, T657S, T657A, T657N, R661K, R661H, R661Q, R661E, Q695E, Q695N, Q695D, H698F, H698Q, H698N, S719A, S719T, S719V, L720I, L720V, L720M, L720T, L720N, L720F, L720A, H721F, H721Y, H721Q, H721E, H721N, H721D, H721S, H721T, K735R, K735Q, K735H, K735M, K735L, L738I, L738V, L738M, L738T, L738N, L738F, L738A, K742R, K742Q, K742H, K742M, K742L, M751L, M751I, M751V, M751T, M751K, R765K, R765H, R765Q, S777A, S777N, S777D, E779Q, E779H, E779D, E779N, D850N, D850S, D850A, Q894E, Q894N, Q894S, E910Q, E910S, L911V, L911A, F916H, F916L, F916M, F916I, F916A, K918Q, K918R, K918H, K918M, K918L, Q920E, Q920N, Q920S, Q920A, T924S, T924A, R925K, R925H, R925Q, Q926K, Q926E, Q926N, Q926D, K929R, K929H, K929Q, Q933E, Q933H, Q933K, Q933N, R951Q, R951S, S960A, S964A, K968Q, K968H, K968M, K968L, R976K, R976Q, R976H, V982A, V982T, V982L, V982I, Y1013F, Y1013H, G1030A, G1030S, G1030T, G1030V, A1032G, A1032S, A1032T, A1032V, K1085R, K1085Q, K1085H, K1085M, K1085L, M1089L, M1089I, M1089V, M1089T, M1089K, P1090A, P1090G, P1090S, P1090T, P1090V, Q1091D, Q1091S, Q1091E, Q1091H, Q1091K, Q1091N, T1098A, T1098P, T1098S, T1098V, E1099, E1099Q, E1099D, E1099N, E1099H, V1100A, V1100G, V1100T, V1100S, V1100I, V1100L, T1102A, T1102P, T1102S, T1102V, G1103A, G1103P, G1103S, F1105Y, F1105H, F1105L, F1105I, F1105V, F1105W, K1107R, K1107Q, K1107M, E1108Q, E1108D, E1108N, S1109A, S1109T, S1109N, S1109D, K1113R, K1113Q, K1113H, K1113M, K1113L, R1114K, R1114Q, R1114H, N1115D, N1115A, N1115S, S1116A, D1117N, D1117S, D1117A, K1118Q, K1118H, K1118M, K1118L, K1123R, K1123Q, K1123H, K1123M, K1123L, K1123S, K1124R, K1124Q, K1124H, K1124M, K1124L, W1126Y, W1126S, F1134Y, F1134H, F1134L, F1134I, F1134V, F1134W, D1135N, D1135S, D1135A, R1171K, R1171H, R1171Q, R1171E, K1197R, K1197Q, K1197H, K1197M, K1197L, K1211R, K1211Q, K1211H, K1211M, K1211L, S1216A, E1219Q, E1219D, E1219N, E1243Q, E1243D, E1243N, E1243S, E1243A, R1279K, R1279H, R1279Q, R1279E, D1284N, D1284S, D1284A, P1301S, P1301G, H1311Y, H1311S, R1333K, R1333Q, R1333H, K1334R, K1334Q, K1334M, K1334L, R1335K, R1335Q, R1335H, T1337A, T1337S, S1338A, H1349F, H1349Y, H1349Q, H1349E, H1349N, H1349D, H1349S, H1349T, Y1356F, Y1356W, Y1356H, Y1356Q, Y1356E, Y1356N, Y1356D, Y1356S, Y1356T, T1358A, T1358P, T1358S, and T1358V.

Preferably, the at least one amino acid alteration occur in a position important for Class-II Cas9 protein-protein interactions and is selected from the group consisting of P176A, P176G, P176S, P176T, P176V, S179A, S179G, S179T, S179V, S179P, E223Q, E223D, E223N, E223H, E223S, E223A, E223I, E223L, E223T, E223V, E223P, E223K, E223Y, E223W, E223F, E223G, E223C, E223R, E260Q, E260S, D261N, D261S, T310A, T310G, P316G, P316D, P411A, P411G, Q413N, Q413A, I414V, I414A, Y430F, Y430V, F432H, F432V, R457K, R457H, R457Q, R457E, R457S, N459D, N459E, N459Q, N459H, N459S, N459T, N459A, R461K, R461H, R461Q, R461E, R461S, R557K, R557S, R653H, R653N, E910Q, E910S, L911V, L911A, R951Q, R951S, K1123R, K1123Q, K1123H, K1123M, K1123L, K1123S, P1301S, P1301G, H1311Y, and H1311S.

Also preferably, the at least one amino acid alteration occur in a position important for interactions between a Class-II Cas9 protein and one or more gRNA and is selected from the group consisting of S29A, S29G, S29T, S29V, K31R, K31Q, K31H, K31M, K31L, F32Y, F32H, F32H, F32L, F32I, F32M, L35V, L35A, L35T, L35I, L35M, L35F, L35H, K44R, K44Q, K44H, K44M, K44L, E57Q, K44D, K44N, K44H, K44M, K44L, N46D, N46S, N46T, N46Q, E57Q, E57D, E57N, E57H, T62S, T62A, T62V, T62P, R63K, R63H, R63Q, R63E, K65R, K65H, K65Q, K65M, K65L, R66K, R66H, R66Q, R66E, R69K, R69H, R69Q, R69E, R70K, R70H, R70Q, R70E, R71K, R71H, R71Q, R71E, Y72F, Y72H, Y72L, Y72M, Y72I, Y72W, R74K, R74H, R74Q, R74E, R75K, R75H, R75Q, R75E, R78K, R78H, R78Q, R78E, S104A, S104A, S104T, S104V, F105H, F105L, F105M, F105I, F105Y, F105W, V107S, V107A, V107I, V107L, V107T, E108Q, E108D, E108N, R115K, R115H, R115Q, R115E, H116F, H116Y, H116Q, H116E, H116N, H116D, H116S, H116T, V126I, V126L, V126A, V126T, V126S, H129F, H129Y, H129Q, H129E, H129N, H129D, H129S, H129T, Y136F, Y136H, Y136L, Y136M, Y136I, Y136W, H160F, H160Y, H160Q, H160E, H160N, H160D, H160S, H160T, K163R, K163H, K163Q, K163E, K163M, K163L, F164Y, F164H, F164M, F164L, F164I, F164W, R165K, R165H, R165Q, R165E, Y325F, Y325H, Y325L, Y325M, Y325I, Y325W, H328F, H328Y, H328Q, H328E, H328N, H328D, H328S, H328T, H329F, H329Y, H329Q, H329E, H329N, H329D, H329S, H329T, K336R, K336H, K336M, K336L, R340K, R340H, R340Q, R340E, Y347F, Y347H, Y347M, Y347L, Y347I, F351H, F351Y, F351M, F351L, F351I, F351W, F352H, F352Y, F352M, F352L, F352I, F352W, I363F, I363L, I363M, I363V, I363A, I363T, D364E, D364Q, D364H, D364N, D364S, D364T, R403K, R403H, R403Q, R403E, I448L, I448M, I448V, I448A, P454A, P454G, P454S, P454T, P454V, L455I, L455V, L455M, L455T, L455N, L455F, L455A, R457K, R457H, R457Q, R457E, R457S, N459D, N459E, N459Q, N459H, N459S, N459T, N459A, S460A, S460G, S460T, S460V, R461K, R461H, R461Q, R461E, R461S, F462Y, F462H, F462L, F462I, F462V, F462W, R467K, R467H, R467Q, R467E, T472A, T472P, T472S, T472V, Y515F, Y515H, Y515M, Y515L, R661K, R661H, R661Q, R661E, S719A, S719T, S719V, L720I, L720V, L720M, L720T, L720N, L720F, L720A, H721F, H721Y, H721Q, H721E, H721N, H721D, H721S, H721T, K735R, K735Q, K735H, K735M, K735L, L738I, L738V, L738M, L738T, L738N, L738F, L738A, K742R, K742Q, K742H, K742M, K742L, M751L, M751I, M751V, M751T, M751K, S777A, S777N, S777D, E779Q, E779H, E779D, E779N, D850N, D850S, D850A, K918Q, K918R, K918H, K918M, K918L, Q933E, Q933H, Q933K, Q933N, V982A, V982T, V982L, V982I, K1085R, K1085Q, K1085H, K1085M, K1085L, M1089L, M1089I, M1089V, M1089T, M1089K, P1090A, P1090G, P1090S, P1090T, P1090V, Q1091D, Q1091S, Q1091E, Q1091H, Q1091K, Q1091N, T1098A, T1098P, T1098S, T1098V, E1099, E1099Q, E1099D, E1099N, E1099H, V1100A, V1100G, V1100T, V1100S, V1100I, V1100L, T1102A, T1102P, T1102S, T1102V, G1103A, G1103P, G1103S, F1105Y, F1105H, F1105L, F1105I, F1105V, F1105W, E1108Q, E1108D, E1108N, K1113R, K1113Q, K1113H, K1113M, K1113L, K1123R, K1123Q, K1123H, K1123M, K1123L, K1123S, K1124R, K1124Q, K1124H, K1124M, K1124L, F1134Y, F1134H, F1134L, F1134I, F1134V, F1134W, R1171K, R1171H, R1171Q, R1171E, K1197R, K1197Q, K1197H, K1197M, K1197L, K1211R, K1211Q, K1211H, K1211M, K1211L, R1279K, R1279H, R1279Q, R1279E, H1349F, H1349Y, H1349Q, H1349E, H1349N, H1349D, H1349S, H1349T, Y1356F, Y1356W, Y1356H, Y1356Q, Y1356E, Y1356N, Y1356D, Y1356S, Y1356T, T1358A, T1358P, T1358S, and T1358V.

Also preferably, the at least one amino acid alteration occur in a position important for interactions between a Class-II Cas9 protein and one or more genome target sequence and is selected from the group consisting of T13S, T13A, N14D, N14S, N14A, S55A, D269N, D269S, D269A, Y450H, Y450L, Y450F, F491H, F491L, M495K, M495Q, M495L, N497D, N497Q, N497E, N497S, G582A, G582S, G582T, G582V, T657S, T657A, T657N, R661K, R661H, R661Q, R661E, Q695E, Q695N, Q695D, H698F, H698Q, H698N, R765K, R765H, R765Q, S777A, S777N, S777D, Q894E, Q894N, Q894S, F916H, F916L, F916M, F916I, F916A, K918Q, K918R, K918H, K918M, K918L, Q920E, Q920N, Q920S, Q920A, T924S, T924A, R925K, R925H, R925Q, Q926K, Q926E, Q926N, Q926D, K929R, K929H, K929Q, S960A, S964A, K968Q, K968H, K968M, K968L, R976K, R976Q, R976H, Y1013F, Y1013H, G1030A, G1030S, G1030T, G1030V, A1032G, A1032S, A1032T, A1032V, K1107R, K1107Q, K1107M, E1108Q, E1108D, E1108N, S1109A, S1109T, S1109N, S1109D, R1114K, R1114Q, R1114H, N1115D, N1115A, N1115S, S1116A, D1117N, D1117S, D1117A, K1118Q, K1118H, K1118M, K1118L, D1135N, D1135S, D1135A, S1216A, E1219Q, E1219D, E1219N, E1243Q, E1243D, E1243N, E1243S, E1243A, D1284N, D1284S, D1284A, R1333K, R1333Q, R1333H, K1334R, K1334Q, K1334M, K1334L, R1335K, R1335Q, R1335H, T1337A, T1337S, and S1338A.

Also preferably, the at least one amino acid alteration occur in a position important for Class-II Cas9 protein-protein interactions and/or in a position important for interactions between a Class-II Cas9 protein and one or more gRNA selected from the group consisting of S29A, S29G, S29T, S29V, K31R, K31Q, K31H, K31M, K31L, F32Y, F32H, F32H, F32L, F32I, F32M, L35V, L35A, L35T, L35I, L35M, L35F, L35H, K44R, K44Q, K44H, K44M, K44L, E57Q, K44D, K44N, K44H, K44M, K44L, N46D, N46S, N46T, N46Q, E57Q, E57D, E57N, E57H, T62S, T62A, T62V, T62P, R63K, R63H, R63Q, R63E, K65R, K65H, K65Q, K65M, K65L, R66K, R66H, R66Q, R66E, R69K, R69H, R69Q, R69E, R70K, R70H, R70Q, R70E, R71K, R71H, R71Q, R71E, Y72F, Y72H, Y72L, Y72M, Y72I, Y72W, R74K, R74H, R74Q, R74E, R75K, R75H, R75Q, R75E, R78K, R78H, R78Q, R78E, S104A, S104A, S104T, S104V, F105H, F105L, F105M, F105I, F105Y, F105W, V107S, V107A, V107I, V107L, V107T, E108Q, E108D, E108N, R115K, R115H, R115Q, R115E, H116F, H116Y, H116Q, H116E, H116N, H116D, H116S, H116T, V126I, V126L, V126A, V126T, V126S, H129F, H129Y, H129Q, H129E, H129N, H129D, H129S, H129T, Y136F, Y136H, Y136L, Y136M, Y136I, Y136W, H160F, H160Y, H160Q, H160E, H160N, H160D, H160S, H160T, K163R, K163H, K163Q, K163E, K163M, K163L, F164Y, F164H, F164M, F164L, F164I, F164W, R165K, R165H, R165Q, R165E, P176A, P176G, P176S, P176T, P176V, S179A, S179G, S179T, S179V, S179P, E223Q, E223D, E223N, E223H, E223S, E223A, E223I, E223L, E223T, E223V, E223P, E223K, E223Y, E223W, E223F, E223G, E223C, E223R, E260Q, E260S, D261N, D261S, T310A, T310G, P316G, P316D, Y325F, Y325H, Y325L, Y325M, Y325I, Y325W, H328F, H328Y, H328Q, H328E, H328N, H328D, H328S, H328T, H329F, H329Y, H329Q, H329E, H329N, H329D, H329S, H329T, K336R, K336H, K336M, K336L, R340K, R340H, R340Q, R340E, Y347F, Y347H, Y347M, Y347L, Y347I, F351H, F351Y, F351M, F351L, F351I, F351W, F352H, F352Y, F352M, F352L, F352I, F352W, I363F, I363L, I363M, I363V, I363A, I363T, D364E, D364Q, D364H, D364N, D364S, D364T, R403K, R403H, R403Q, R403E, P411A, P411G, Q413N, Q413A, I414V, I414A, Y430F, Y430V, F432H, F432V, I448L, I448M, I448V, I448A, P454A, P454G, P454S, P454T, P454V, L455I, L455V, L455M, L455T, L455N, L455F, L455A, R457K, R457H, R457Q, R457E, R457S, N459D, N459E, N459Q, N459H, N459S, N459T, N459A, S460A, S460G, S460T, S460V, R461K, R461H, R461Q, R461E, R461S, F462Y, F462H, F462L, F462I, F462V, F462W, R467K, R467H, R467Q, R467E, T472A, T472P, T472S, T472V, Y515F, Y515H, Y515M, Y515L, R557K, R557S, R653H, R653N, R661K, R661H, R661Q, R661E, S719A, S719T, S719V, L720I, L720V, L720M, L720T, L720N, L720F, L720A, H721F, H721Y, H721Q, H721E, H721N, H721D, H721S, H721T, K735R, K735Q, K735H, K735M, K735L, L738I, L738V, L738M, L738T, L738N, L738F, L738A, K742R, K742Q, K742H, K742M, K742L, M751L, M751I, M751V, M751T, M751K, S777A, S777N, S777D, E779Q, E779H, E779D, E779N, D850N, D850S, D850A, E910Q, E910S, L911V, L911A, K918Q, K918R, K918H, K918M, K918L, Q933E, Q933H, Q933K, Q933N, R951Q, R951S, V982A, V982T, V982L, V982I, K1085R, K1085Q, K1085H, K1085M, K1085L, M1089L, M1089I, M1089V, M1089T, M1089K, P1090A, P1090G, P1090S, P1090T, P1090V, Q1091D, Q1091S, Q1091E, Q1091H, Q1091K, Q1091N, T1098A, T1098P, T1098S, T1098V, E1099, E1099Q, E1099D, E1099N, E1099H, V1100A, V1100G, V1100T, V1100S, V1100I, V1100L, T1102A, T1102P, T1102S, T1102V, G1103A, G1103P, G1103S, F1105Y, F1105H, F1105L, F1105I, F1105V, F1105W, E1108Q, E1108D, E1108N, K1113R, K1113Q, K1113H, K1113M, K1113L, K1123R, K1123Q, K1123H, K1123M, K1123L, K1123S, K1124R, K1124Q, K1124H, K1124M, K1124L, F1134Y, F1134H, F1134L, F1134I, F1134V, F1134W, R1171K, R1171H, R1171Q, R1171E, K1197R, K1197Q, K1197H, K1197M, K1197L, K1211R, K1211Q, K1211H, K1211M, K1211L, R1279K, R1279H, R1279Q, R1279E, P1301S, P1301G, H1311Y, H1311S, H1349F, H1349Y, H1349Q, H1349E, H1349N, H1349D, H1349S, H1349T, Y1356F, Y1356W, Y1356H, Y1356Q, Y1356E, Y1356N, Y1356D, Y1356S, Y1356T, T1358A, T1358P, T1358S, and T1358V.

Also preferably, the at least one amino acid alteration occur in a position important for Class-II Cas9 protein-protein interactions and/or in a position important for interactions between a Class-II Cas9 protein and one or more genome target sequence selected from the group consisting of T13S, T13A, N14D, N14S, N14A, S55A, P176A, P176G, P176S, P176T, P176V, S179A, S179G, S179T, S179V, S179P, E223Q, E223D, E223N, E223H, E223S, E223A, E223I, E223L, E223T, E223V, E223P, E223K, E223Y, E223W, E223F, E223G, E223C, E223R, E260Q, E260S, D261N, D261S, D269N, D269S, D269A, T310A, T310G, P316G, P316D, P411A, P411G, Q413N, Q413A, I414V, I414A, Y430F, Y430V, F432H, F432V, Y450H, Y450L, Y450F, R457K, R457H, R457Q, R457E, R457S, N459D, N459E, N459Q, N459H, N459S, N459T, N459A, R461K, R461H, R461Q, R461E, R461S, F491H, F491L, M495K, M495Q, M495L, N497D, N497Q, N497E, N497S, R557K, R557S, G582A, G582S, G582T, G582V, R653H, R653N, T657S, T657A, T657N, R661K, R661H, R661Q, R661E, Q695E, Q695N, Q695D, H698F, H698Q, H698N, R765K, R765H, R765Q, S777A, S777N, S777D, Q894E, Q894N, Q894S, E910Q, E910S, L911V, L911A, F916H, F916L, F916M, F916I, F916A, K918Q, K918R, K918H, K918M, K918L, Q920E, Q920N, Q920S, Q920A, T924S, T924A, R925K, R925H, R925Q, Q926K, Q926E, Q926N, Q926D, K929R, K929H, K929Q, R951Q, R951S, S960A, S964A, K968Q, K968H, K968M, K968L, R976K, R976Q, R976H, Y1013F, Y1013H, G1030A, G1030S, G1030T, G1030V, A1032G, A1032S, A1032T, A1032V, K1107R, K1107Q, K1107M, E1108Q, E1108D, E1108N, S1109A, S1109T, S1109N, S1109D, R1114K, R1114Q, R1114H, N1115D, N1115A, N1115S, S1116A, D1117N, D1117S, D1117A, K1118Q, K1118H, K1118M, K1118L, K1123R, K1123Q, K1123H, K1123M, K1123L, K1123S, D1135N, D1135S, D1135A, S1216A, E1219Q, E1219D, E1219N, E1243Q, E1243D, E1243N, E1243S, E1243A, D1284N, D1284S, D1284A, P1301S, P1301G, H1311Y, H1311S, R1333K, R1333Q, R1333H, K1334R, K1334Q, K1334M, K1334L, R1335K, R1335Q, R1335H, T1337A, T1337S, and S1338A.

Also preferably, the at least one amino acid alteration occur in a position important for interactions between a Class-II Cas9 protein and one or more gRNA and/or in a position important for interactions between a Class-II Cas9 protein and one or more genome target sequence selected from the group consisting of T13S, T13A, N14D, N14S, N14A, S29A, S29G, S29T, S29V, K31R, K31Q, K31H, K31M, K31L, F32Y, F32H, F32H, F32L, F32I, F32M, L35V, L35A, L35T, L35I, L35M, L35F, L35H, K44R, K44Q, K44H, K44M, K44L, E57Q, K44D, K44N, K44H, K44M, K44L, N46D, N46S, N46T, N46Q, S55A, E57Q, E57D, E57N, E57H, T62S, T62A, T62V, T62P, R63K, R63H, R63Q, R63E, K65R, K65H, K65Q, K65M, K65L, R66K, R66H, R66Q, R66E, R69K, R69H, R69Q, R69E, R70K, R70H, R70Q, R70E, R71K, R71H, R71Q, R71E, Y72F, Y72H, Y72L, Y72M, Y72I, Y72W, R74K, R74H, R74Q, R74E, R75K, R75H, R75Q, R75E, R78K, R78H, R78Q, R78E, S104A, S104A, S104T, S104V, F105H, F105L, F105M, F105I, F105Y, F105W, V107S, V107A, V107I, V107L, V107T, E108Q, E108D, E108N, R115K, R115H, R115Q, R115E, H116F, H116Y, H116Q, H116E, H116N, H116D, H116S, H116T, V126I, V126L, V126A, V126T, V126S, H129F, H129Y, H129Q, H129E, H129N, H129D, H129S, H129T, Y136F, Y136H, Y136L, Y136M, Y136I, Y136W, H160F, H160Y, H160Q, H160E, H160N, H160D, H160S, H160T, K163R, K163H, K163Q, K163E, K163M, K163L, F164Y, F164H, F164M, F164L, F164I, F164W, R165K, R165H, R165Q, R165E, D269N, D269S, D269A, Y325F, Y325H, Y325L, Y325M, Y325I, Y325W, H328F, H328Y, H328Q, H328E, H328N, H328D, H328S, H328T, H329F, H329Y, H329Q, H329E, H329N, H329D, H329S, H329T, K336R, K336H, K336M, K336L, R340K, R340H, R340Q, R340E, Y347F, Y347H, Y347M, Y347L, Y347I, F351H, F351Y, F351M, F351L, F351I, F351W, F352H, F352Y, F352M, F352L, F352I, F352W, I363F, I363L, I363M, I363V, I363A, I363T, D364E, D364Q, D364H, D364N, D364S, D364T, R403K, R403H, R403Q, R403E, I448L, I448M, I448V, I448A, Y450H, Y450L, Y450F, P454A, P454G, P454S, P454T, P454V, L455I, L455V, L455M, L455T, L455N, L455F, L455A, R457K, R457H, R457Q, R457E, R457S, N459D, N459E, N459Q, N459H, N459S, N459T, N459A, S460A, S460G, S460T, S460V, R461K, R461H, R461Q, R461E, R461S, F462Y, F462H, F462L, F462I, F462V, F462W, R467K, R467H, R467Q, R467E, T472A, T472P, T472S, T472V, F491H, F491 L, M495K, M495Q, M495L, N497D, N497Q, N497E, N497S, Y515F, Y515H, Y515M, Y515L, G582A, G582S, G582T, G582V, T657S, T657A, T657N, R661K, R661H, R661Q, R661E, Q695E, Q695N, Q695D, H698F, H698Q, H698N, S719A, S719T, S719V, L720I, L720V, L720M, L720T, L720N, L720F, L720A, H721F, H721Y, H721Q, H721E, H721N, H721D, H721S, H721T, K735R, K735Q, K735H, K735M, K735L, L738I, L738V, L738M, L738T, L738N, L738F, L738A, K742R, K742Q, K742H, K742M, K742L, M751L, M751I, M751V, M751T, M751K, R765K, R765H, R765Q, S777A, S777N, S777D, E779Q, E779H, E779D, E779N, D850N, D850S, D850A, Q894E, Q894N, Q894S, F916H, F916L, F916M, F916I, F916A, K918Q, K918R, K918H, K918M, K918L, Q920E, Q920N, Q920S, Q920A, T924S, T924A, R925K, R925H, R925Q, Q926K, Q926E, Q926N, Q926D, K929R, K929H, K929Q, Q933E, Q933H, Q933K, Q933N, S960A, S964A, K968Q, K968H, K968M, K968L, R976K, R976Q, R976H, V982A, V982T, V982L, V982I, Y1013F, Y1013H, G1030A, G1030S, G1030T, G1030V, A1032G, A1032S, A1032T, A1032V, K1085R, K1085Q, K1085H, K1085M, K1085L, M1089L, M1089I, M1089V, M1089T, M1089K, P1090A, P1090G, P1090S, P1090T, P1090V, Q1091D, Q1091S, Q1091E, Q1091H, Q1091K, Q1091N, T1098A, T1098P, T1098S, T1098V, E1099, E1099Q, E1099D, E1099N, E1099H, V1100A, V1100G, V1100T, V1100S, V1100I, V1100L, T1102A, T1102P, T1102S, T1102V, G1103A, G1103P, G1103S, F1105Y, F1105H, F1105L, F1105I, F1105V, F1105W, K1107R, K1107Q, K1107M, E1108Q, E1108D, E1108N, S1109A, S1109T, S1109N, S1109D, K1113R, K1113Q, K1113H, K1113M, K1113L, R1114K, R1114Q, R1114H, N1115D, N1115A, N1115S, S1116A, D1117N, D1117S, D1117A, K1118Q, K1118H, K1118M, K1118L, K1123R, K1123Q, K1123H, K1123M, K1123L, K1123S, K1124R, K1124Q, K1124H, K1124M, K1124L, F1134Y, F1134H, F1134L, F1134I, F1134V, F1134W, D1135N, D1135S, D1135A, R1171K, R1171H, R1171Q, R1171E, K1197R, K1197Q, K1197H, K1197M, K1197L, K1211R, K1211Q, K1211H, K1211M, K1211L, S1216A, E1219Q, E1219D, E1219N, E1243Q, E1243D, E1243N, E1243S, E1243A, R1279K, R1279H, R1279Q, R1279E, D1284N, D1284S, D1284A, R1333K, R1333Q, R1333H, K1334R, K1334Q, K1334M, K1334L, R1335K, R1335Q, R1335H, T1337A, T1337S, S1338A, H1349F, H1349Y, H1349Q, H1349E, H1349N, H1349D, H1349S, H1349T, Y1356F, Y1356W, Y1356H, Y1356Q, Y1356E, Y1356N, Y1356D, Y1356S, Y1356T, T1358A, T1358P, T1358S, and T1358V.

More preferably, the at least one amino acid alteration is selected from the group consisting of S104A, F105H, V107S, P176S, S179A, E223A, N235NGSGAGGSY, E260Q, D261N, T310A, T310G, P316G, P316D, P411A, P411G, Q413N, Q413A, I414V, I414A, Y430F, Y430V, F432H, F432V, R457K, N459S, P475S, W476H, R557K, R653H, Q739S, E910S, L911A, R951Q, K1123S, W1126S, and H1311Y; most preferably, the at least one amino acid alteration is selected from the group consisting of S104A, F105H, V107S, P176S, S179A, E223A, E260Q, D261N, T310A, T310G, P316G, P316D, P411A, P411G, Q413N, Q413A, I414V, I414A, Y430F, Y430V, F432H, F432V, R457K, N459S, P475S, W476H, R557K, R653H, Q739S, E910S, L911A, R951Q, K1123S, W1126S, and H1311Y.

Even more preferably, the at least one amino acid alteration include N235NGSGAGGSY; S104A, F105H, and V107S; P176S and S179A; E223A; T310A and P316G; P411A, Q413N, and I414V; P411A, Q413N, I414V, R457K, and N495S; Y430F and F432H; R457K and N495S; P475S and W476H; R557K; R653H; R951Q; S104A, F105H, V107S, P176S, S179A, E223S, E260Q, D261N, R653H, Q739S, and H1311Y; S104A, F105H, V107S, E260Q, D261N, T310A, P316G, Y430F, F432H, Q739S, and H1311Y; S104A, F105H, V107S, T310A, P316G, Q739S, and R951Q; S104A, F105H, V107S, E260Q, D261N, Y430F, F432H, and Q739S; S104A, F105H, V107S, Q739S, R951Q, and H1311Y; S104A, F105H, V107S, P411A, Q413N, and I414V; S104A, F105H, V107S, P411A, Q413N, I414V, R457K, and N459S; T310G P316D; P411G, Q413A, and I414A; Y430V and F432V; E910S and L911A; and K1123S and W1126S; most preferably the at least one amino acid alteration include S104A, F105H, and V107S; P176S and S179A; E223A; T310A and P316G; P411A, Q413N, and I414V; P411A, Q413N, I414V, R457K, and N495S; Y430F and F432H; R457K and N495S; P475S and W476H; R557K; R653H; R951Q; S104A, F105H, V107S, P176S, S179A, E223S, E260Q, D261N, R653H, Q739S, and H1311Y; S104A, F105H, V107S, E260Q, D261N, T310A, P316G, Y430F, F432H, Q739S, and H1311Y; S104A, F105H, V107S, T310A, P316G, Q739S, and R951Q; S104A, F105H, V107S, E260Q, D261N, Y430F, F432H, and Q739S; S104A, F105H, V107S, Q739S, R951Q, and H1311Y; S104A, F105H, V107S, P411A, Q413N, and I414V; S104A, F105H, V107S, P411A, Q413N, I414V, R457K, and N459S; T310G P316D; P411G, Q413A, and I414A; Y430V and F432V; E910S and L911A; and K1123S and W1126S.

Preferably the at least one amino acid alteration include N235NGSGAGGSY.

Preferably the at least one amino acid alteration include S104A, F105H, and V107S.

Preferably the at least one amino acid alteration include P176S and S179A.

Preferably the at least one amino acid alteration include E223A.

Preferably the at least one amino acid alteration include T310A and P316G.

Preferably the at least one amino acid alteration include P411A, Q413N, and I414V.

Preferably the at least one amino acid alteration include P411A, Q413N, I414V, R457K, and N495S.

Preferably the at least one amino acid alteration include Y430F and F432H.

Preferably the at least one amino acid alteration include R457K and N495S.

Preferably the at least one amino acid alteration include P475S and W476H.

Preferably the at least one amino acid alteration include R557K.

Preferably the at least one amino acid alteration include R653H.

Preferably the at least one amino acid alteration include R951Q.

Preferably the at least one amino acid alteration include S104A, F105H, V107S, P176S, S179A, E223S, E260Q, D261N, R653H, Q739S, and H1311Y.

Preferably the at least one amino acid alteration include S104A, F105H, V107S, E260Q, D261N, T310A, P316G, Y430F, F432H, Q739S, and H1311Y.

Preferably the at least one amino acid alteration include S104A, F105H, V107S, T310A, P316G, Q739S, and R951Q.

Preferably the at least one amino acid alteration include S104A, F105H, V107S, E260Q, D261N, Y430F, F432H, and Q739S.

Preferably the at least one amino acid alteration include S104A, F105H, V107S, Q739S, R951Q, and H1311Y.

Preferably the at least one amino acid alteration include S104A, F105H, V107S, P411A, Q413N, and I414V.

Preferably the at least one amino acid alteration include S104A, F105H, V107S, P411A, Q413N, I414V, R457K, and N459S.

Preferably the at least one amino acid alteration include T310G and P316D.

Preferably the at least one amino acid alteration include P411G, Q413A, and I414A.

Preferably the at least one amino acid alteration include Y430V and F432V.

Preferably the at least one amino acid alteration include E910S and L911A.

Preferably the at least one amino acid alteration include K1123S and W1126S; The temperature-sensitive variants of the invention are characterized by a permissive temperature or temperature range, where the variant behaves as the wildtype protein and is able to form a complex with one or more gRNA and one or more genome target sequences of interest, and by a restrictive temperature or temperature range, where the variant adopts the mutant phenotype and is unable to form a complex with one or more gRNA and one or more genome target sequence of interest. The permissive and restrictive temperatures or temperature ranges depend on thermostability of a given tsCas9 and the temperature requirements of a given host cell. In general, the permissive and restrictive temperature are in the range from 25° C. to 45° C. Preferably, the permissive and restrictive temperature or temperature range should be separated by at least 1° C. Alternatively, the permissive and restrictive temperature ranges may overlap.

Depending on the host cell, the permissive temperature may be a temperature selected from 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.

The permissive temperature may also be a temperature range selected from 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.

The permissive temperature may also be a temperature range selected from 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.

The permissive temperature may also be a temperature range selected from 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.

The permissive temperature may also be a temperature range selected from 25-31° C., 26-32° C., 27-33° C., 28-34° C., 29-35° C., 30-36° C., 31-37° C., 32-38° C., 33-39° C., 34-40° C., 35-41° C., 36-42° C., 37-43° C., 38-44° C., and 39-45° C.

The permissive temperature may also be a temperature range selected from 25-32° C., 26-33° C., 27-34° C., 28-35° C., 29-36° C., 30-37° C., 31-38° C., 32-39° C., 33-40° C., 34-41° C., 35-42° C., 36-43° C., 37-44° C., and 38-45° C.

The permissive temperature may also be a temperature range selected from 25-33° C., 26-34° C., 27-35° C., 28-36° C., 29-37° C., 30-38° C., 31-39° C., 32-40° C., 33-41° C., 34-42° C., 35-43° C., 36-44° C., and 37-45° C.

The permissive temperature may also be a temperature range selected from 25-34° C., 26-35° C., 27-36° C., 28-37° C., 29-38° C., 30-39° C., 31-40° C., 32-41° C., 33-42° C., 34-43° C., 35-44° C., and 36-45° C.

The permissive temperature may also be a temperature range selected from 25-35° C., 26-36° C., 27-37° C., 28-38° C., 29-39° C., 30-40° C., 31-41° C., 32-42° C., 33-43° C., 34-44° C., and 35-45° C.

For Bacillus host cells, the permissive temperature range is selected from 30-32° C., 30-33° C., 29-32° C., 30-34° C., 29-33° C., 30-35° C., 29-34° C., 30-36° C., 28-34° C., 31-35° C., 28-35° C., 26-34° C., and 27-34° C. Preferably, the permissive temperature range is selected from 30-32° C., 29.5-32.5° C., 29-33° C., 28.5-33.5° C., 28-34° C., 27.5-34.5° C., 27-35° C., 26.5-35.5° C., and 26-36° C. More preferably, the permissive temperature range is selected from 29-33° C., 28.5-33.5° C., 28-34° C., 27.5-34.5° C., and 27-35° C. Even more preferably, the permissive temperature range is selected from 28.5-33.5° C., 28-34° C., and 27.5-34.5° C. Most preferably, the permissive temperature range is 28-34° C.

Depending on the host cell, the restrictive temperature may be a temperature selected from 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., and 45° C.

The restrictive temperature may also be a temperature range selected from 25-28° C., 26-29° C., 27-30° C., 28-31° C., 29-32° C., 30-33° C., 31-34° C., 32-35° C., 33-36° C., 34-37° C., 35-38° C., 36-39° C., 37-40° C., 38-41° C., 39-42° C., 40-43° C., 41-44° C., and 42-45° C.

The restrictive temperature may also be a temperature range selected from 25-29° C., 26-30° C., 27-31° C., 28-32° C., 29-33° C., 30-34° C., 31-35° C., 32-36° C., 33-37° C., 34-38° C., 35-39° C., 36-40° C., 37-41° C., 38-42° C., 39-43° C., 40-44° C., 41-45° C.

The restrictive temperature may also be a temperature range selected from 25-30° C., 26-31° C., 27-32° C., 28-33° C., 29-34° C., 30-35° C., 31-36° C., 32-37° C., 33-38° C., 34-39° C., 35-40° C., 36-41° C., 37-42° C., 38-42° C., 39-44° C., and 40-45° C.

The restrictive temperature may also be a temperature range selected from 25-31° C., 26-32° C., 27-33° C., 28-34° C., 29-35° C., 30-36° C., 31-37° C., 32-38° C., 33-39° C., 34-40° C., 35-41° C., 36-42° C., 37-43° C., 38-44° C., and 39-45° C.

The restrictive temperature may also be a temperature range selected from 25-32° C., 26-33° C., 27-34° C., 28-35° C., 29-36° C., 30-37° C., 31-38° C., 32-39° C., 33-40° C., 34-41° C., 35-42° C., 36-43° C., 37-44° C., and 38-45° C.

The restrictive temperature may also be a temperature range selected from 25-33° C., 26-34° C., 27-35° C., 28-36° C., 29-37° C., 30-38° C., 31-39° C., 32-40° C., 33-41° C., 34-42° C., 35-43° C., 36-44° C., and 37-45° C.

The restrictive temperature may also be a temperature range selected from 25-34° C., 26-35° C., 27-36° C., 28-37° C., 29-38° C., 30-39° C., 31-40° C., 32-41° C., 33-42° C., 34-43° C., 35-44° C., and 36-45° C.

The restrictive temperature may also be a temperature range selected from 25-35° C., 26-36° C., 27-37° C., 28-38° C., 29-39° C., 30-40° C., 31-41° C., 32-42° C., 33-43° C., 34-44° C., and 35-45° C.

For Bacillus host cells, the restrictive temperature range is selected from 38-40° C., 38-41° C., 37-40° C., 38-42° C., 37-41° C., 37-42° C., 36-40° C., 36-41° C., 36-42° C., and 35-43° C. Preferably, the restrictive temperature range is selected from 39-40° C., 38.5-41.5° C., 38-42° C., 37.5-42° C., 38-42.5° C., 36.5-41.5° C., 36.5-42° C., 37-42° C., 37-42.5° C., and 36-43° C. More preferably, the restrictive temperature range is selected from 38-42° C., 37.5-42° C., 38-42.5° C., 36.5-41.5° C., 36.5-42° C., 37-42° C., 37-42.5° C., 36-43° C., and 36-44° C. Even more preferably, the restrictive temperature range is selected from 36.5-41.5° C., 36.5-42° C., 37-42° C., and 37-42.5° C. Most preferably, the restrictive temperature range is 37-42° C.

In a preferred embodiment, the permissive temperature is 28-34° C., and the restrictive temperature is 37-42° C.

Also in a preferred embodiment, the permissive temperature is 28-34° C., and the restrictive temperature is 37-44° C.

Also in a preferred embodiment, the permissive temperature is 27-36° C., and the restrictive temperature is 37-42° C.

Also in a preferred embodiment, the permissive temperature is 27-36° C., and the restrictive temperature is 37-44° C.

A tsCas9 of the present invention may further be characterized by a dissociative temperature or temperature range, where a complex formed between the tsCas9, one or more gRNA, and the corresponding one or more genome target sequence of interest dissociates. The dissociative temperature may be a range selected from 40-50° C., 41-50° C., 42-50° C., 43-50° C., 44-50° C., and 45-50° C. Preferably, the dissociative temperature range is selected from 41-50° C., 42-50° C., and 43-50° C. More preferably, the dissociative temperature range is selected from 41-50° C., 41.5-50° C., 42-50° C., 42.5-50° C., and 43-50° C. Even more preferably, the dissociative temperature range is selected from 41.5-50° C., 42-50° C., and 42.5-50° C. Most preferably, the dissociative temperature range is 42-50° C.

### Preparation of Temperature-Sensitive Variants

The present invention also relates to methods for obtaining a temperature-sensitive variant of a Class-II Cas9 protein, comprising: (a) introducing into a parent Class-II Cas9 protein an alteration at one or more (e.g., several) positions important for Class-II Cas9 protein stability or for stability of a complex formed between a Class-II Cas9 protein, one or more gRNA, and one or more genome target sequence, and, optionally, (b) recovering the variant.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to polynucleotides encoding a temperature-sensitive variant of a Class-II Cas9 protein. In a preferred embodiment, the polynucleotide encoding a tsCas9 has a sequence identity to the mature polypeptide coding sequence of SEQ ID NO:1 of at least 80%, e.g., at least 85%, at least 90%, at least 91%, at least 92%, at least 93%, at least 94%, at least 95%, at least 96%, at least 97%, at least 98%, at least 99%, or 100%.

The present invention also relates to isolated polynucleotides encoding a temperature-sensitive variant of a Class-II Cas9 protein. The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Streptococcus, or a related organism and thus, for example, may be an allelic or species variant of the polypeptide encoding region of the polynucleotide.

Modification of a polynucleotide encoding a temperature-sensitive variant of the present invention may be necessary for synthesizing polypeptides substantially similar to such variant. The term “substantially similar” to the polypeptide refers to non-naturally occurring forms of the polypeptide.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase 1, Trichoderma reesei xylanase 1l, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome. For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMB1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier.

The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of a variant of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The bacterial host cell may also be any Lactobacillus cell including, but not limited to, Lactobacillus acidophilus, Lactobacillus amylovorus, Lactobacillus brevis, Lactobacillus (para)casei, Lactobacillus cellobiosus, Lactobacillus crispatus, Lactobacillus curvatus, Lactobacillus delbrueckii subsp. bulgaricus, L. delbrueckii subsp. lactis, Lactobacillus fermentum, Lactobacillus gallinarum, Lactobacillus gasseri, Lactobacillus helveticus, Lactobacillus johnsonii, Lactobacillus plantarum, Lactobacillus reuteri, Lactobacillus rhamnosus, and Lactobacillus salivarius cell.

The bacterial host cell may also be any Lactococcus cell including, but not limited to, Lactococcus chungangensis, Lactococcus formosensis, Lactococcus fujiensis, Lactococcus garvieae, Lactococcus lactis, Lactococcus piscium, Lactococcus plantarum, Lactococcus raffinolactis, and Lactococcus taiwanensis.

The bacterial host cell may also be any Escherichia cell. In a preferred embodiment, the bacterial host cell is E. coli.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell. For eukaryotic host cells, the temperature-sensitive variants of the invention must comprise at least one nuclear localization sequence (NLS) fused to the tsCas9 protein in order to ensure its localization in the nucleus of the cell; preferably the Simian virus 40 (SV40) T antigen nuclear localization signal (NLS) is fused on the N- and/or C-terminus of the tsCas9 protein.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as S. cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods for Inducing or Repressing Expression

The present invention also relates to methods for inducing or repressing expression of one or more genome target sequence of interest.

In a preferred aspect, the present invention relates to a method of inducing expression of one or more genome target sequence of interest, the method comprising the steps of:
a) providing a host cell of the invention, said host cell further comprising one or more suitable gRNA and one or more genome target sequence of interest;
b) cultivating the host cell at a dissociative temperature of the tsCas9, whereby a complex formed in the host cell by the tsCas9 with the one or more suitable gRNA and the one or more genome target sequence of interest disassociates; and subsequently
c) lowering the temperature to a restrictive temperature of the tsCas9 and cultivating the host cell, whereby expression of the one or more target sequence is induced.

The method for inducing expression of one or more genome target sequence of interest may also comprise the following step after providing the host cell and before step (b) of: cultivating the host cell at a permissive temperature of the tsCas9, whereby a complex is formed in the host cell by the tsCas9, the one or more suitable gRNA and the one or more genome target sequence of interest, whereby expression of the one or more genome target sequence is repressed.

It may be of interest to only transiently induce the expression of the one or more genome target sequence of interest. Accordingly, in a preferred embodiment, the method of inducing expression of one or more genome target sequence of interest may further comprise the additional step of:
d) increasing the temperature to a permissive temperature of the tsCas9, wherein a complex is formed in the host cell by the tsCas9, the one or more suitable gRNA, and the one or more genome target sequence of interest, and cultivating the host cell, whereby expression of the one or more genome target sequence of interest is repressed.

In another preferred aspect, the present invention relates to a method of repressing one or more genome target sequence of interest, the method comprising the steps of:
a) providing a host cell of the invention, said host cell further comprising one or more suitable gRNA and one or more genome target sequence of interest;
b) cultivating the host cell at a restrictive temperature of the tsCas9, wherein the one or more genome target sequence of interest is expressed; and subsequently
c) lowering the temperature to a permissive temperature of the tsCas9, whereby a complex is formed in the host cell by the tsCas9, the one or more suitable gRNA and the one or more genome target sequence of interest, and whereby expression of the one or more genome target sequence is repressed.

The method for repressing expression of one or more genome target sequence of interest may also comprise the following step after providing the host cell and before step (b) of: cultivating the host cell at a dissociative temperature of the tsCas9 to ensure that no complex is formed in the host cell between the tsCas9, the one or more gRNA and the one or more genome target sequence of interest.

It may be of interest to only transiently repress the expression of the one or more genome target sequence of interest. Accordingly, in a preferred embodiment, the method of repressing expression of one or more genome target sequence of interest may further comprise the additional steps of:
d) raising the temperature to a dissociative temperature of the tsCas9, whereby the complex disassociates; and subsequently
e) lowering the temperature to a restrictive temperature of the tsCas9, whereby expression of the one or more target sequence is induced.

For methods of inducing or repressing expression, the host cells is preferably a Bacillus host cell; preferably the host cell is selected from the group of Bacillus species consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis; more preferably the host cell is Bacillus licheniformis.

For Bacillus host cells, the permissive temperature is a range selected from 30-32° C., 30-33° C., 29-32° C., 30-34° C., 29-33° C., 30-35° C., 29-34° C., 30-36° C., 28-34° C., 31-35° C., 28-35° C., 26-34° C., and 27-34° C. Preferably, the permissive temperature range is selected from 30-32° C., 29.5-32.5° C., 29-33° C., 28.5-33.5° C., 28-34° C., 27.5-34.5° C., 27-35° C., 26.5-35.5° C., and 26-36° C. More preferably, the permissive temperature range is selected from 29-33° C., 28.5-33.5° C., 28-34° C., 27.5-34.5° C., and 27-35° C. Even more preferably, the permissive temperature range is selected from 28.5-33.5° C., 28-34° C., and 27.5-34.5° C. Most preferably, the permissive temperature range is 28-34° C.

For Bacillus host cells, the restrictive temperature is a range selected from 38-40° C., 38-41° C., 37-40° C., 38-42° C., 37-41° C., 37-42° C., 36-40° C., 36-41° C., 36-42° C., and 35-43° C. Preferably, the restrictive temperature range is selected from 39-40° C., 38.5-41.5° C., 38-42° C., 37.5-42° C., 38-42.5° C., 36.5-41.5° C., 36.5-42° C., 37-42° C., 37-42.5° C., and 36-43° C. More preferably, the restrictive temperature range is selected from 38-42° C., 37.5-42° C., 38-42.5° C., 36.5-41.5° C., 36.5-42° C., 37-42° C., 37-42.5° C., 36-43° C., and 36-44° C. Even more preferably, the restrictive temperature range is selected from 36.5-41.5° C., 36.5-42° C., 37-42° C., and 37-42.5° C., Most preferably, the restrictive temperature range is 37-42° C.,

In a preferred embodiment, the permissive temperature is 28-34° C., and the restrictive temperature is 37-42° C.

For methods of inducing or repressing expression, the tsCas9 has a dissociative temperature that is a range selected from 40-50° C., 41-50° C., 42-50° C., 43-50° C., 44-50° C., and 45-50° C. Preferably, the dissociative temperature range is selected from 41-50° C., 42-50° C., and 43-50° C. More preferably, the dissociative temperature range is selected from 41-50° C., 41.5-50° C., 42-50° C., 42.5-50° C., and 43-50° C. Even more preferably, the dissociative temperature range is selected from 41.5-50° C., 42-50° C., and 42.5-50° C. Most preferably, the dissociative temperature range is 42-50° C.

### Guide-RNA

The gRNA in CRISPR-Cas9 genome editing constitutes the re-programmable part that makes the system so versatile. In the natural S. pyogenes system, the gRNA is actually a complex of two RNA polynucleotides, a first crRNA containing about 20 nucleotides that determine the specificity of the Cas9 enzyme and the tracr RNA which hybridizes to the crRNA to form an RNA complex that interacts with Cas9 (see Jinek et al., 2012, A programmable dual-RNA-guided DNA endonuclease in adaptive bacterial immunity, Science 337: 816-821). The terms crRNA and tracrRNA are used interchangeably with the terms tracr-mate RNA and tracr RNA herein.

Since the discovery of the CRISPR-Cas9 system single polynucleotide gRNAs have been developed and successfully applied just as effectively as the natural two part gRNA complex.

In a preferred embodiment, the single gRNA or RNA complex comprises a first RNA comprising 20 or more nucleotides that are at least 85% complementary to and capable of hybridizing to the one or more genome target sequence; preferably the 20 or more nucleotides are at least 90%, 95%, 97%, 98%, 99% or even 100% complementary to and capable of hybridizing to the one or more genome target sequence.

In another preferred embodiment, the Bacillus host cell comprises a single gRNA comprising the first and second RNAs in the form of a single polynucleotide and wherein the tracr mate sequence and the tracr sequence form a stem-loop structure when hybridized with each other.

### Genome Target Sequence

The one or more genome target sequence of interest that may be either induced or repressed is at least 20 nucleotides in length in order to allow its hybridization to the corresponding 20 nucleotide sequence of the gRNA. The at least one genome target sequence can be located anywhere in the genome but will often be within a coding sequence or open reading frame.
the one or more genome target sequence of interest should be flanked by a functional PAM sequence for a Class-II Cas9 protein. For an overview of PAM sequences, see, for example, Shah et al, 2013, Protospacer recognition motifs, RNA Biol. 10(5): 891-899.

Preferably, the one or more genome target sequence is comprised in an open reading frame encoding a polypeptide or in a promoter region. Also preferably, the one or more genome target sequence of interest encode one or more enzyme selected from the group consisting of hydrolase, isomerase, ligase, lyase, oxidoreductase, or a transferase; preferably the one or more enzyme is an alpha-amylase, alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, mannosidase, mutanase, oxidase, pectinolytic enzyme, peroxidase, phosphodiesterase, phytase, polyphenoloxidase, proteolytic enzyme, ribonuclease, transglutaminase, and xylanase.

Preferably, if the microorganism host cell is a Bacillus host cell, the one or more genome target sequence of interest to be repressed comprises the mecA and/or the yjbH gene or homologues thereof, as exemplified herein. Other preferred genome target sequences of interest to be repressed comprise protease-encoding genes, especially cytosolic, secreted or membrane-bound proteases that, if expressed, may degrade a recombinantly produced polypeptide.

### Methods of Production of Temperature-Sensitive Variants

The present invention also relates to methods of producing a variant of the invention, comprising: (a) cultivating a host cell of the present invention under conditions suitable for expression of the variant; and (b) recovering the variant.

The host cells are cultivated in a nutrient medium suitable for production of the variant using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the variant to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the such variants. These detection methods include, but are not limited to, use of specific antibodies, formation of an enzyme product, or disappearance of an enzyme substrate. For example, an enzyme assay may be used to determine the activity of the variant.

The variant may be recovered using methods known in the art. For example, the variant may be recovered from the nutrient medium by conventional procedures including, but not limited to, collection, centrifugation, filtration, extraction, spray-drying, evaporation, or precipitation.

The variant may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure variants.

In an alternative aspect, the variant is not recovered, but rather a host cell of the present invention expressing the variant is used as a source of the variant.

The present invention is further described by the following examples that should not be construed as limiting to the scope of the invention.

### EXAMPLES

### Materials and Methods

### Materials

Chemicals used as buffers and substrates were commercial products of at least reagent grade.

PCR amplifications were performed using standard textbook procedures, employing a commercial thermocycler and either Ready-To-Go PCR beads, Phusion polymerase, or RED-TAQ polymerase from commercial suppliers.

LB agar: See EP 0 506 780.

LBPSG agar plates contains LB agar supplemented with phosphate (0.01 M K3PO4), glucose (0.4%), and starch (0.5%); See EP 0 805 867 B1.

TY (liquid broth medium; See WO 94/14968, p. 16.

Oligonucleotide primers were obtained from DNA technology, Aarhus, Denmark. DNA manipulations (plasmid and genomic DNA preparation, restriction digestion, purification, ligation, DNA sequencing) was performed using standard textbook procedures with commercially available kits and reagents.

Ligation mixtures were in some cases amplified in an isothermal rolling circle amplification reaction, using the TempliPhi kit from GE Healthcare.

DNA was introduced into B. subtilis rendered naturally competent, either using a two step procedure (Yasbin et al., 1975, J. Bacteriol. 121: 296-304.), or a one step procedure, in which cell material from an agar plate was resuspended in Spizisen 1 medium (WO 2014/052630), 12 ml shaken at 200 rpm for appr. 4 hours at 37° C., DNA added to 400 microliter aliquots, and these further shaken 150 rpm for 1 hour at the desired temperature before plating on selective agar plates.

DNA was introduced into B. licheniformis by conjugation from B. subtilis, essentially as previously described (EP2029732 B1), using a modified B. subtilis donor strain PP3724, containing pLS20, wherein the methylase gene M.bli1904II (US20130177942) is expressed from a triple promoter at the amyE locus, the pBC16-derived orf beta and the B. subtilis comS gene (and a kanamycin resistance gene) are expressed from a triple promoter at the air locus (making the strain D-alanine requiring), and the B. subtilis comS gene (and a cat gene) are expressed from a triple promoter at the pel locus.

Bacillus subtilis JA1343: JA1343 is a sporulation negative derivative of PL1801 (WO 2005042750). Part of the gene spoIIAC has been deleted to obtain the sporulation negative phenotype.

All of the constructions described in the examples were assembled from synthetic DNA fragments ordered from GeneArt—ThermoFisher Scientific. The fragments were assembled by sequence overlap extension (SOE) as described in the examples.

The temperature-sensitive plasmids used in this patent was incorporated into the genome of B. licheniformis by chromosomal integration and excision according to the method previously described (U.S. Pat. No. 5,843,720). B. licheniformis transformants containing plasmids were grown on LBPG selective medium with erythromycin at 50° C. to force integration of the vector at identical sequences to the chromosome. Desired integrants were chosen based on their ability to grow on LBPG+erythromycin selective medium at 50° C. Integrants were then grown without selection in LBPG medium at 37° C. to allow excision of the integrated plasmid. Cells were plated on LBPG plates and screened for erythromycin-sensitivity. The sensitive clones were checked for correct integration of the desired construct.

Genomic DNA was prepared from several erythromycin sensitive isolates above accordingly to the method previously described (Pitcher et. al, supra) or by using the commercial available QI-Aamp DNA Blood Kit from Qiagen.

### Standard Fed-Batch Cultivation Procedure

All growth media were sterilized by methods known in the art. Unless otherwise described, tap water was used. The ingredient concentrations referred to in the below recipes are before any inoculation.

First inoculum medium: SSB4 agar. Soy peptone SE50MK (DMV) 10 g/l; sucrose 10 g/l;

Di-Sodiumhydrogenphosphate, 2H20 5 g/l; Potassiumdihydrogenphosphate 2 g/l; Citric acid 0.2 g/l; Vitamins (Thiamin-hydrochlorid 1 1.4 mg/l; Riboflavin 0.95 mg/l; Nicotinic amide 7.8 mg/l; Calcium D-pantothenate 9.5 mg/l; Pyridoxal-HCl 1.9 mg/l; D-biotin 0.38 mg/l; Folic acid 2.9 mg/l); Trace metals (MnS04, H20 9.8 mg/l; FeS04, 7H20 39.3 mg/l; CuS04, 5H20 3.9 mg/l; ZnS04, 7H20 8.2 mg/l); Agar 25 g/l. Use of deionized water. pH adjusted to pH 7.3 to 7.4 with NaOH.

Transfer buffer. M-9 buffer (deionized water is used): Di-Sodiumhydrogenphosphate, 2H20 8.8 g/l; Potassiumdihydrogenphosphate 3 g/l; Sodium Chloride 4 g/l; Magnesium sulphate, 7H20 0.2 g/l.

Inoculum shake flask medium (concentration is before inoculation): PRK-50: 1 10 g/l soy grits; Di-Sodiumhydrogenphosphate, 2H20 5 g/l; pH adjusted to 8.0 with NaOH/H3P04 before sterilization.

Make-up medium (concentration is before inoculation): Tryptone (Casein hydrolysate from Difco) 30 g/l; Magnesium sulphate, 7H20 4 g/l; Di-Potassiumhydrogenphosphate 7 g/l; Di-Sodiumhydrogenphosphate, 2H₂0 7 g/l; Di-Ammoniumsulphate 4 g/l; Potassiumsulphate 5 g/l; Citric acid 0.78 g/l; Vitamins (Thiamin-hydrochlorid 34.2 mg/l; Riboflavin 2.8 mg/l; Nicotinic amide 23.3 mg/l; Calcium D-pantothenate 28.4 mg/l; Pyridoxal-HCl 5.7 mg/l; D-biotin 1.1 mg/l; Folic acid 2.5 mg/l); Trace metals (MnS04, H20 39.2 mg/l; FeS04, 7H20 157 mg/l; CuS04, 5H20 15.6 mg/l; ZnS04, 7H20 32.8 mg/l); Antifoam (SB2121) 1.25 ml/l; pH adjusted to 6.0 with NaOH/H3P04 before sterilization.

Feed medium: Sucrose 708 g/l;

Inoculum steps: First the strain was grown on SSB-4 agar slants 1 day at 37° C. The agar was then washed with M-9 buffer, and the optical density (OD) at 650 nm of the resulting cell suspension was measured. The inoculum shake flask (PRK-50) was inoculated with an inoculum of OD (650 nm)×ml cell suspension=0.1. The shake flask was incubated at 37° C. at 300 rpm for 20 hr. The fermentation in the main fermentor (fermentation tank) was started by inoculating the main fermentor with the growing culture from the shake flask. The inoculated volume was 11% of the make-up medium (80 ml for 720 ml make-up media).

Standard lab fermentors were used equipped with a temperature control system, pH control with ammonia water and phosphoric acid, dissolved oxygen electrode to measure oxygen saturation through the entire fermentation.

Fermentation parameters: Temperature: 38° C.; The pH was kept between 6.8 and 7.2 using ammonia water and phosphoric acid; Control: 6.8 (ammonia water); 7.2 phosphoric acid;

Aeration: 1.5 liter/min/kg broth weight.

Agitation: 1500 rpm.

Feed strategy: 0 hr. 0.05 g/min/kg initial broth after inoculation; 8 hr. 0.156 g/min/kg initial broth after inoculation; End 0.156 g/min/kg initial broth after inoculation.

Experimental setup: The cultivation was run for five days with constant agitation, and the oxygen tension was followed on-line in this period. The different strains were compared side by side.

Measurements of GFP fluorescence was performed on full broth cell cultures. The culture was diluted and measured directly on a SpectraMax M2 from Molecular Devices

### Strains

PP3724: Containing pLS20, wherein the methylase gene M.bli1904II (US20130177942) is expressed from a triple promoter at the amyE locus, the pBC16-derived orf beta and the B. subtilis comS gene (and a kanamycin resistance gene) are expressed from a triple promoter at the air locus (making the strain D-alanine requiring), and the B. subtilis comS gene (and a cat gene) are expressed from a triple promoter at the pel locus.

PL1801: This strain is the B. subtilis DN 1885 with disrupted aprE and nprE genes encoding the alkaline protease and neutral protease, respectively (Diderichsen, B., Wedsted, U., Hedegaard, L., Jensen, B. R., Sjøholm, C. (1990) Cloning of aldB, which encodes alpha-acetolactate decarboxylase, an exoenzyme from Bacillus brevis J. Bacterid., 172, 4315-4321).

A164: This strain is a B. subtilis wild type isolate (ATCC 6051 a).

JA1343: This strain is the B. subtilis PL1801 with a disrupted spoIIAC gene (sigF). The genotype is: aprE, nprE, amyE, spoIIAC.

SJ4671: This B. licheniformis strain has two copies of the amyL gene integrated at the original amyL locus on the chromosome. The two copies are inserted in opposite directions so that transcription of the two copies are antiparallel. The copies are spaced by approximately 2.5 Kb originating from non-functional DNA of the B. subtilis chromosome (U.S. Pat. No. 6,100,063).

SJ6026: This B. licheniformis strain has four copies of the amyL gene integrated at the amyL, xyl and gnt loci.

MOL2173: This B. licheniformis strain has four copies of the amyL gene integrated at the amyL, xyl and gnt loci and one additional prsA gene inserted at the mprL locus.

MOL2212: This B. licheniformis strain is an rifampicin resistant isolate of MOL2173.

PP2307: This B. subtilis strain is JA1343 with an expression cassette inserted into the pel gene holding P3 promoter driving comS and a kanamycin marker.

PP5007: This B. licheniformis strain is MOL2212 where the native catL gene is inactivated.

The strain is chloramphenicol sensitive.

PP5021: This B. licheniformis strain is PP5007 where an expression cassette holding the cas9d, gDNA(P4199) and cat is inserted at the forD locus.

PP5168: This strain is the B. subtilis strain JA1343 with the gfp gene, gDNA(P4199) and spec marker inserted at the pel locus.

PP5336: This strain is the B. subtilis strain PP5168 with the cas9d gene, gDNA(GFP) and cat marker inserted at the amyE locus.

PP5200: This strain is B. subtilis strain PP5168 with the cas9d gene, gDNA(GFP) and cat marker inserted at the pel locus.

C1-C48: These strains are B. subtilis strain PP5168 with the temperature sensitive cas9d gene variants, gDNA(GFP) and cat marker inserted at the pel locus.

PP5360: This B. licheniformis strain is PP5007 where an expression cassette holding the temperature sensitive cas9d variant C30, gDNA(P4199) and cat gene is inserted at the forD locus.

PP5541: This B. licheniformis strain is PP5007 where an expression cassette holding the temperature sensitive cas9d variant C25, gDNA(P4199) and cat gene is inserted at the forD locus.

PP5542: This B. licheniformis strain is PP5007 where an expression cassette holding the temperature sensitive cas9d variant C33, gDNA(P4199) and cat gene is inserted at the forD locus.

BKQ3746: This B. licheniformis strain is PP5007 where an expression cassette holding the temperature sensitive cas9d variant C38, gDNA(P4199) and cat gene is inserted at the forD locus.

BKQ3748: This B. licheniformis strain is PP5007 where an expression cassette holding the temperature sensitive cas9d variant C40, gDNA(P4199) and cat gene is inserted at the forD locus.

BKQ3750: This B. licheniformis strain is PP5007 where an expression cassette holding the temperature sensitive cas9d variant C42, gDNA(P4199) and cat gene is inserted at the forD locus.

BKQ3752: This B. licheniformis strain is PP5007 where an expression cassette holding the temperature sensitive cas9d variant C43, gDNA(P4199) and cat gene is inserted at the forD locus.

### Plasmids

pC194: Plasmid isolated from Staphylococcus aureus (Horinouchi and Weisblum, 1982).

pE194: Plasmid isolated from S. aureus (Horinouchi and Weisblum, 1982).

pUB110: Plasmid isolated from (McKenzie et al., 1986)

pPPamyL-attP: Plasmid constructed for this invention in example 6. The plasmid was made by assembly of synthetic sequences to generate a vector holding the: (1) amyL gene encoding the alpha-amylase from B. licheniformis preceded by the cry3A stabilizer for integration (2) the attP and the integrase (int) from TP901-1 described in WO2006042548. The integrase promote integration between the attP site on the plasmid and the attB site on the chromosome of the B. licheniformis host.

### Example 1. Construction of the B. licheniformis Host MOL2212

The full construction of MOL2212 was done in several consecutive steps by a sequential plasmid integrations as described above.

The plasmids for integrations were assembled by PCR amplifications of synthetic DNA. The purified PCR products were used in a subsequent PCR reaction to create a single plasmid using splice overlapping PCR (SOE) using the Phusion Hot Start DNA Polymerase system (Thermo Scientific) as follows. The PCR amplification reaction mixture contained 50 ng of each of the six gel purified PCR products and a thermocycler was used to assemble and amplify the plasmids. The resulting SOE product were used directly for transformation of B. subtilis host JA1343 to establish the plasmids which were later used as vehicles for transfer and integration of DNA into specific loci on the B. licheniformis chromosome.

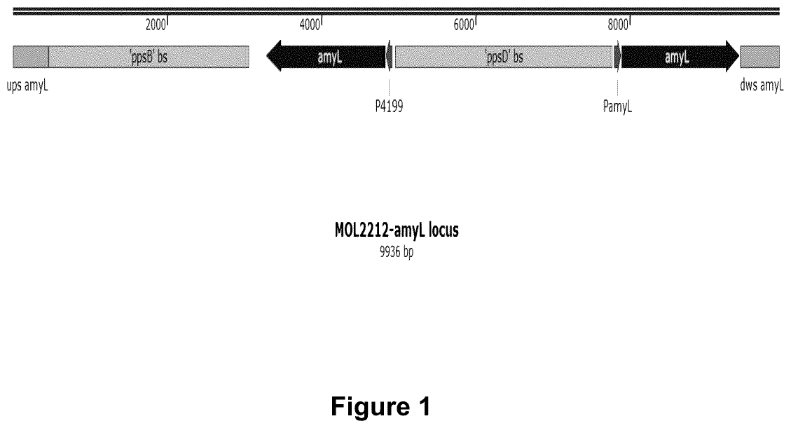

The B. licheniformis strain SJ4671 (U.S. Pat. No. 6,100,063) was used as a host strain for insertion of additional copies of the alpha-amylase gene amyL. The SJ4671 strain already has two copies of the the amyL gene integrated at the original amyL locus on the chromosome. The two copies are inserted in opposite directions so that transcription of the two copies are antiparallel. The copies are spaced by approximately 2.5 Kb originating from non-functional DNA of the B. subtilis chromosome (FIG. 1 and SEQ ID NO:3).

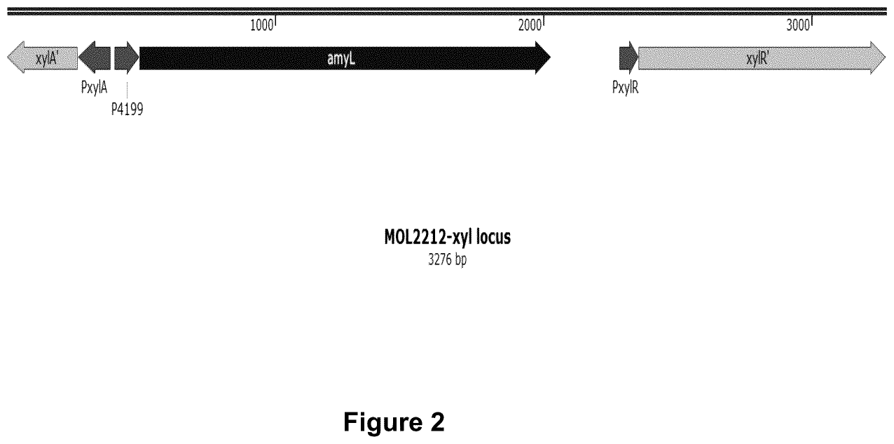

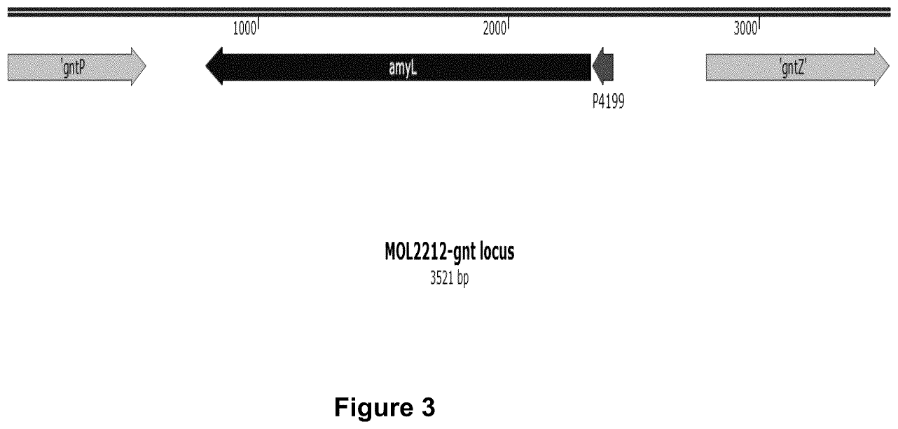

The SJ4671 strain was conjugated with plasmids for further insertions of two more copies of the amyL gene; one copy inserted at the xyl locus (FIG. 2 and SEQ ID NO: 4), and one copy inserted at the gnt locus (FIG. 3 and SEQ ID NO:5). This intermediate strain was named SJ6026 and contained four copies of the alpha-amylase gene, amyL, stably integrated into the chromosome of B. licheniformis.

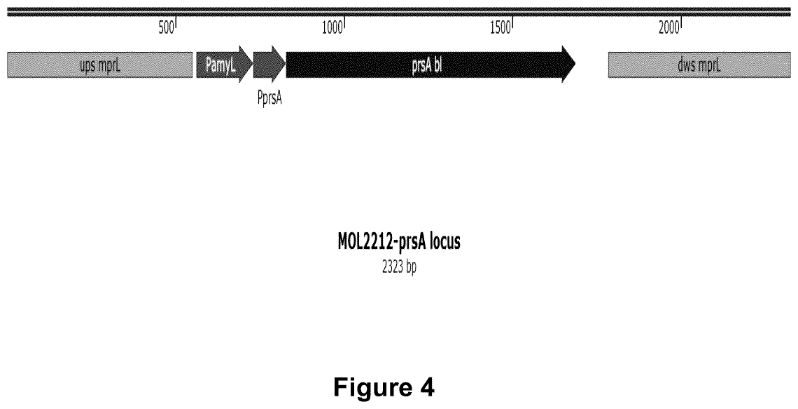

The SJ6026 strain was further engineered by inserting an additional copy of the prsA gene which encodes the chaperone from B. licheniformis. Over-expression of the PrsA chaperone is described in literature to further increase alpha-amylase productivity. The additional prsA gene was inserted at the mprL locus. This insertion lead to over-expression of the PrsA chaperone from B. licheniformis and knock-out of the mprL product—metalloprotease (FIG. 4 and SEQ ID NO:6). This four copy amyL and two copy prsA strain was named MOL2173.

The final strain, named MOL2212, is a derivative of MOL2173 where a spontaneous mutation in the rpoB gene was isolated as a rifampicin resistant strain.

### Example 2. Construction of the B. licheniformis Strains PP5007 and PP5021

The MOL2212 strain was used as a host strain for transformation of a plasmid for inactivation of the native catL gene. A clone was isolated as chloramphenicol sensitive and preserved as PP5007.

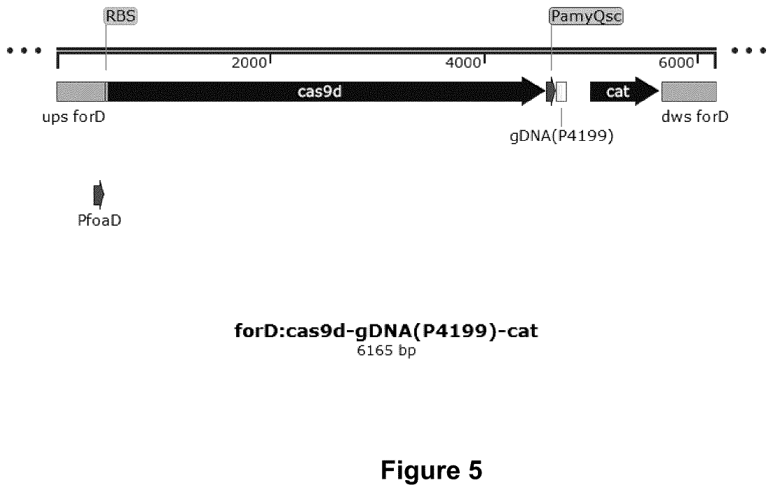

The PP5007 strain was further engineered by transformation of a plasmid which integrate at the forD locus and inserts an expression cassette consisting of the cas9d gene expressed from the forD promoter, the gDNA(P4199) transcribed from the PamyQsc promoter and the cat gene conferring chloramphenicol resistance (FIG. 5 and SEQ ID NO:7). The gDNA(P4199) is transcribed into gRNA(P4199) which directs Cas9d to the P4199 promoter and inhibit transcription. All four amyL gene copies in the final strain PP5021 are expressed from the P4199 promoter and the Cas9d-gRNA complex can potentially bind and inhibit alpha-amylase expression through strong interaction to the P4199 target.

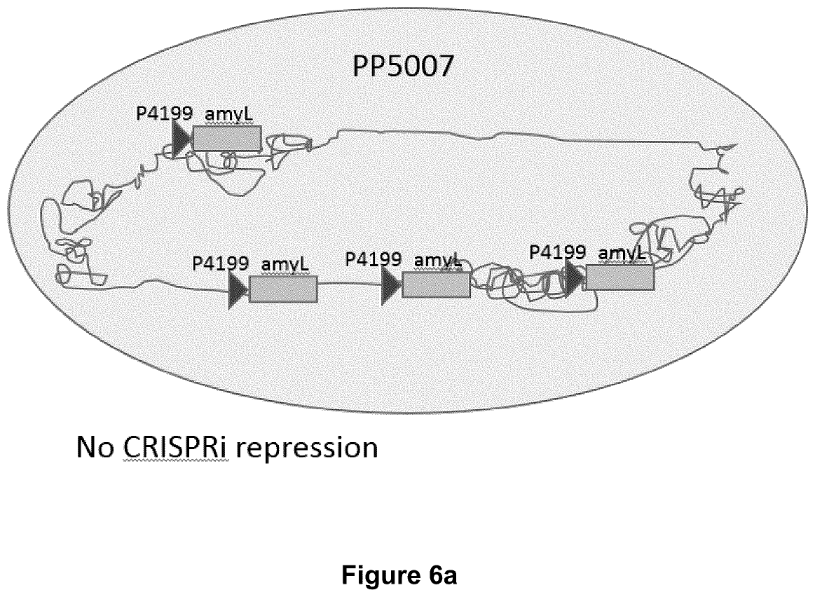

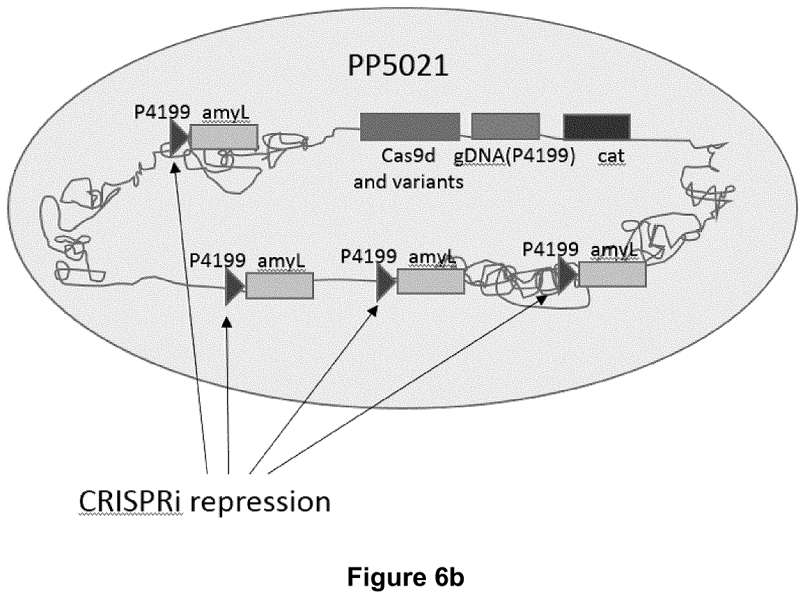

An illustration of the two B. licheniformis strains PP5007 and PP5021 is shown in FIG. 6.

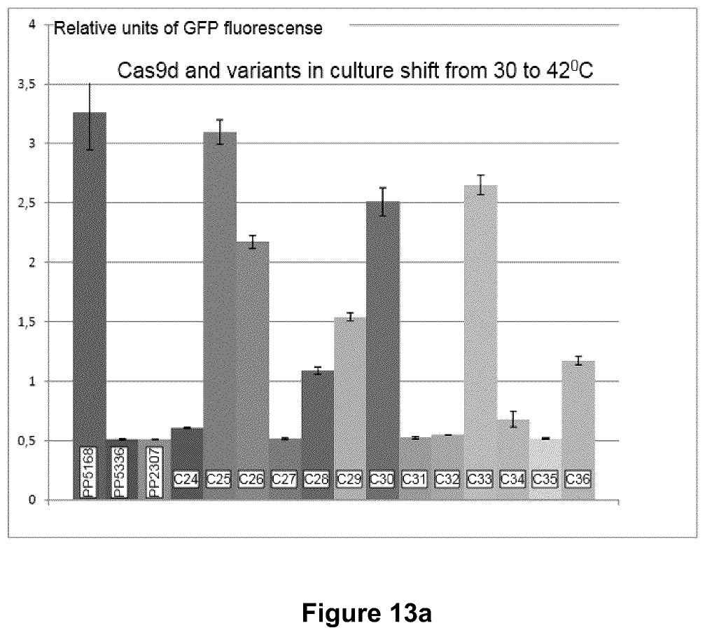

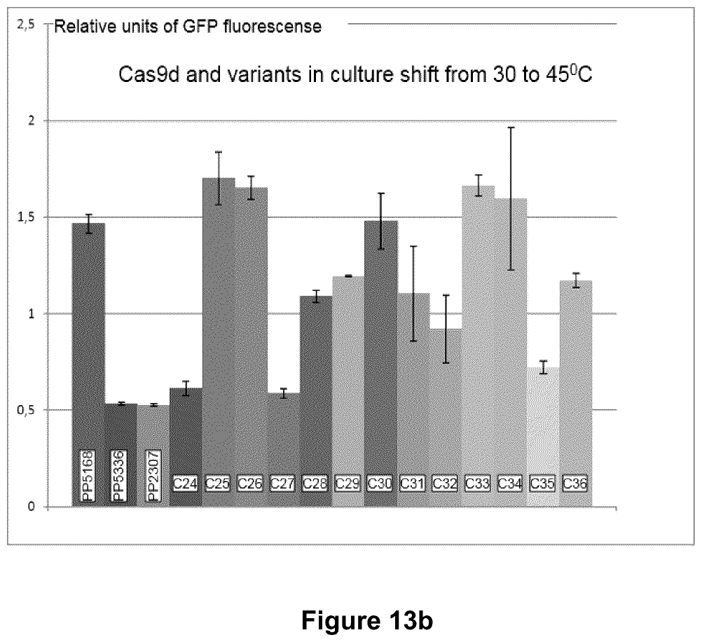

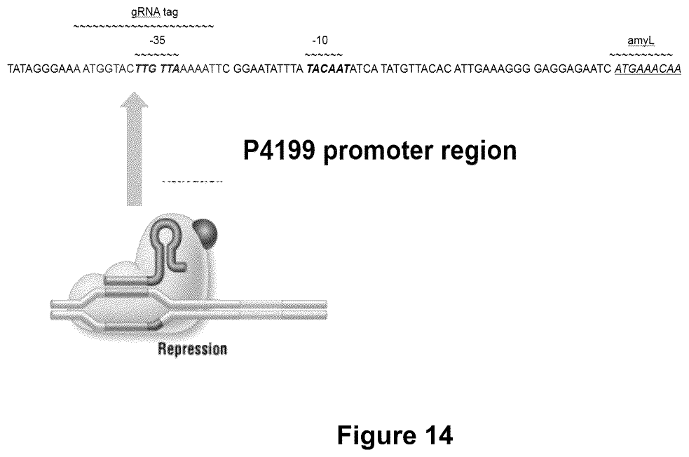

An illustration of the CRISPRi complex binding to the promoter region of P4199 is shown in FIG. 13.

### Example 3. Alpha-Amylase Expression Using the PP5007 and PP5021 Strains

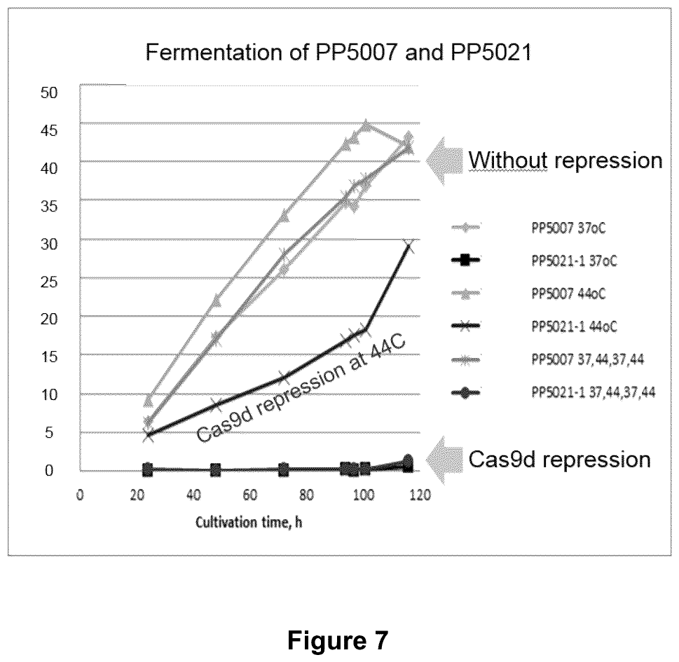

The B. licheniformis strains described in Example 2 was tested with respect to alpha-amylase productivity at different temperatures in fed-batch cultivations as described above. The PP5007 strain has full expression of the amylase from four copies with no inhibition from the CRISPRi complex and can be used as a positive control for the PP5021 where the CRISPRi complex is cloned and active. FIG. 7 clearly shows that the PP5007 strain allow full expression of the amylase enzyme regardless of the cultivation temperature at 37° C. or 44° C. (triangles, stars and diamonds data points). One the contrary, when the strain PP5021 is cultivated at 37° C. the productivity of amylase is close to zero (squares) which shows that the CRISPRi complex formed by the gRNA(P4199), and the Cas9d can inhibit very efficiently the amylase expression from all four copies on the chromosome at 37° C.

Another cultivation with the PP5021 strain was started and kept at the high temperature of 44° C. all the way through the process (X data points). Surprisingly the inhibition of amylase expression is not very efficient and allows considerable production of the amylase to almost to 75% of what is seen for the 37° C. culture experiment. The interpretation of these results is that the CRISPRi inhibition complex at the P4199 promoter cannot be formed as efficient at 44° C. as when the cells are grown at the lower temperature of 37° C. Keeping the temperature at a high level from start to end changes the dynamics, partly prohibits the inhibition and allow expression of amylase under these conditions.

In another experiment with PP5021 the cultivation was started at 37° C. and changed to 44° C. after outgrowth of the culture, then changed back 37° C. and finally up to 44° C. at the end of the cultivation. From the data in FIG. 7 (closed circles) it is evident that changing the temperature between 37° C. and 44° C. does not affect the inhibition of the amylase expression by the CRISPRi complex. The productivity of amylase remains very low all the way through cultivation and the conclusion is that once the CRISPRi inhibition complex is formed at 37° C. it cannot be de-repressed by a sudden change in temperature to 44° C.

With this information at hand we wanted to screen for de-stabilized site-directed variants of Cas9d which had a more suitable temperature induction profile than the current Cas9d protein. The experiments in this example shows that some temperature-sensitivity already exists in the Cas9d complex formation with the gRNA and target DNA but that a relatively high temperature of 44° C. is needed to partly prohibit CRISPRi complex formation and inhibition. At the same time, the currently available Cas9d protein can not be inactivated at high temperature once the CRISPRi inhibition complex is formed and consequently de-repression (induction) will be very difficult during cultivation or at least require very high temperature during cultivation which is incompatible with the growth physiology of the Bacillus organism.

Screening for a temperature-sensitive Cas9d protein will therefore have two goals:
1) Obtaining de-stabilized Cas9d proteins which prohibit CRISPRi complex formation (Cas9d-gRNA) at a lower temperature than the wildtype Cas9d. This will allow a temperature range better suited for—in this case—the physiology of the Bacillus organism during cultivations. In the case of Bacillus, full inhibition at 30-33° C. (functional CRISPRi complex) and full induction at 37° C. (non-functional CRISPRi complex) could be an attractive alternative to the experiments shown for the currently known wildtype (wt) Cas9d described above which need a temperature above 44° C. to be inactivated. For other organisms and processes, alternative temperature ranges could be attractive and likely be designed by site-directed mutagenesis of Cas9d.
2) Obtaining Cas9d protein variants that allow for immediate de-stabilization and de-repression by changing temperature. The current Cas9d molecule forms a complex with the gRNA and target DNA which has a much higher temperature stability once it is established. Screening for Cas9d variants which can be de-stabilized even when already in complex with the gRNA and DNA target would be very valuable. Such Cas9d variants would e.g. allow for a temperature inducible system to be set-up where cell biomass is allowed to build up during cultivation at a temperature where the CRISPRi inhibits expression of a gene and then induce expression by increasing temperature to a level where the CRISPRi complex is de-stabilized.

### Example 4. Chromosomal Integration of GFP and gDNA(P4199) in B. subtilis

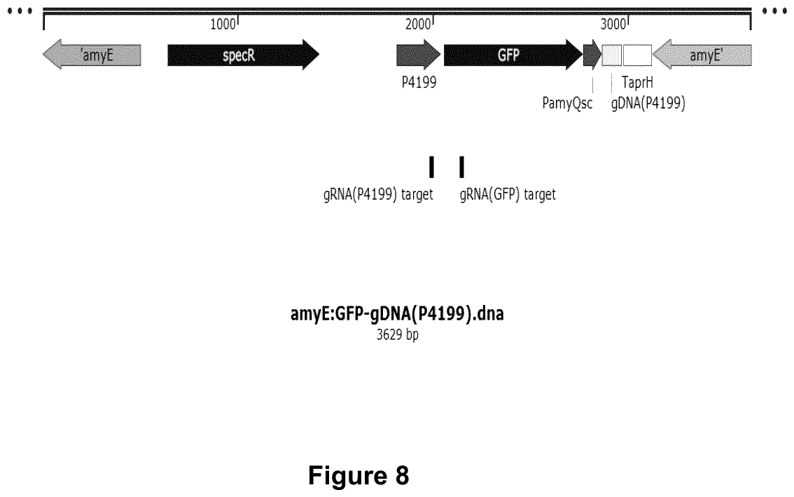

A DNA fragment was inserted at the amyE locus where the GFP gene encoding the green fluorescent protein is expressed from the amyL variant promoter P4199 earlier described in WO1993010249. Furthermore a gDNA(P4199) is expressed from the PamyQ consensus promoter (PamyQsc) described in U.S. Pat. No. 6,255,076. The gDNA(P4199) expresses the gRNA(P4199) with a spacer sequence directing the CRISPRi complex to a sequence on the P4199 promoter. A spectinomycin marker was also included to select for integration. The DNA for integration was ordered as synthetic DNA (GeneArt—ThermoFisher Scientific) and cloned into integration vectors as described in the Material and Methods section. The final map of the amyE locus is shown in FIG. 8. The nucleotide sequence of the locus can be found in SEQ ID NO:8.

The condition for the PCR amplifications is as follows: The respective DNA fragments were amplified by PCR using the Phusion Hot Start DNA Polymerase system (Thermo Scientific). The PCR amplification reaction mixture contained 1 ul (approx. 0.1 ug) of template DNA, 2 ul of sense primer (20 pmol/ul), 2 ul of anti-sense primer (20 pmol/ul), 10 ul of 5×PCR buffer with 7.5 mM MgCl₂, 8 ul of dNTP mix (1.25 mM each), 37 ul water, and 0.5 ul (2 U/ul) DNA polymerase mix. A thermocycler was used to amplify the fragment. The PCR products were purified from a 1.2% agarose gel with 1×TBE buffer using the Qiagen QIAquick Gel Extraction Kit (Qiagen, Inc., Valencia, CA) according to the manufacturer's instructions.

The PCR products were used in a subsequent PCR reactions to create a single plasmid using splice overlapping PCR (SOE) using the Phusion Hot Start DNA Polymerase system (Thermo Scientific) as follows. The PCR amplification reaction mixture contained 50 ng of each of the two gel purified PCR products and the synthetic fragment and a thermocycler was used to assemble the DNA for integration. The resulting SOE product was used directly for transformation to B. subtilis host PP2307 to establish the integration by selecting for spectinomycin.

The final construct has the GFP gene expressed from the P4199 promoter and the gRNA expressed from the PamyQsc promoter on the chromosome (amyE locus). The strain is named PP5168 (FIG. 8).

### Example 5. Chromosomal Integration of cas9d and gDNA(GFP) in B. subtilis

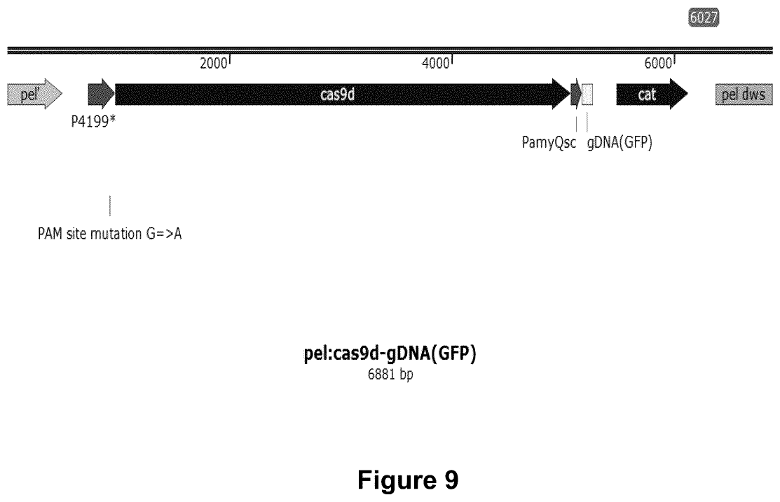

An expression cassette was inserted at the pel locus with the cas9d gene encoding the Cas9d protein (Sander et al., Nature Biotechnology 32, 347-355, 2014). is expressed from the P4199* promoter. The P4199 promoter was earlier described in WO1993010249. The P4199* promoter has a single base change from G to A disrupting a PAM site for CRISPR recognition of the gRNA(P4199). Furthermore a gDNA(GFP) is expressed from the PamyQ consensus promoter (PamyQsc) described in U.S. Pat. No. 6,255,076. The gDNA(GFP) expresses the gRNA(GFP) with a spacer sequence directing the CRISPRi complex to a coding sequence in the GFP gene (see FIG. 9). A Chloramphenicol resistance marker was also included to select for correct integration. The DNA for integration was ordered as synthetic DNA (GeneArt—ThermoFisher Scientific) and cloned into integration vectors as described in the Material and Methods section. The final map of the pel locus is shown in FIG. 9. The nucleotide sequence of the locus can be found in SEQ ID NO:9.

The PCR products were made as described in Example 1 and used in a subsequent PCR reaction to create a single plasmid using splice overlapping PCR (SOE) using the Phusion Hot Start DNA Polymerase system (Thermo Scientific) as follows. The PCR amplification reaction mixture contained 50 ng of each of the two gel purified PCR products and the synthetic fragment and a thermocycler was used to assemble and amplify the DNA for integration. The resulting SOE product was used directly for transformation to B. subtilis PP5168 (Example 4). By selection for chloramphenicol resistance the DNA fragment holding the cas9d gene and the gDNA(GFP) was inserted at the pel locus

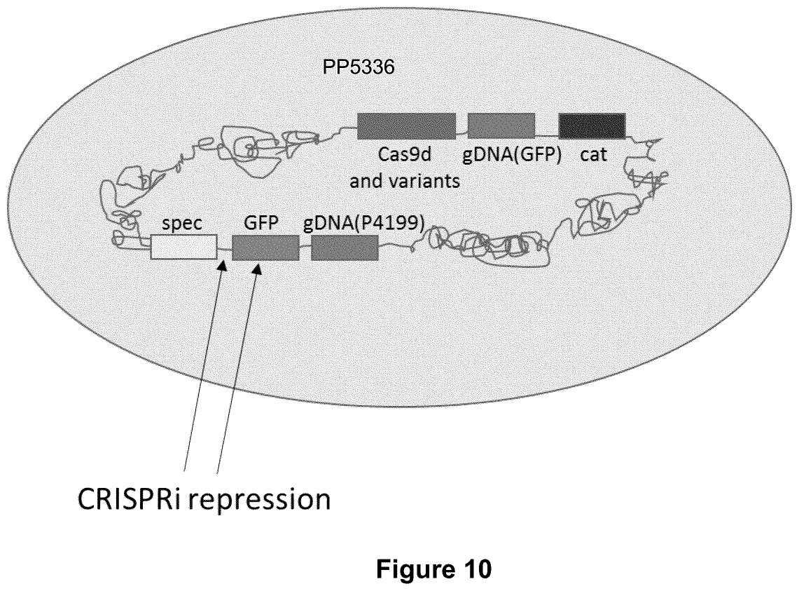

This strain is named PP5336 and expresses both the Cas9d protein, the GFP protein, the gRNA(P4199) and the gRNA(GFP). Please see FIG. 10 for an illustration showing that the GFP expression is blocked both by CRISPR inhibition of the promoter (P4199) and the inside the GFP gene. The strain PP5336 is colorless at temperatures below 45° C. because of the CRISPRi complex (Cas9d-gRNA) inhibition of GFP expression. At temperatures, above 45° C. the strain show green fluorescence because of the inability to form the CRISPRi complex at elevated temperature. The fluorescence properties of PP5336 strain demonstrate that it is a good candidate for screening of variant temperature sensitive variant of Cas9d.

### Example 6. Library of cas9d Variants in B. subtilis

A library of expression cassettes identical to the one described in Example 5 was inserted at the pel locus as described. Different site-directed variants of the Cas9d protein were proposed based on the Cas9 structure (PDB: 5F9R) further described in Example 7. The site-directed variants were introduced by SOE-PCR and cloned as DNA fragments with flanking DNA identity to the pel locus in B. subtilis as earlier described in WO2006042548. The library of different variants were introduced by transformation into competent PP5168 as described in Example 5. The final map of the pel locus is as already shown in FIG. 9 with the only difference being the different amino acid changes in Cas9d. The proposed site-directed variants in Cas9d can be found in Table 1.

TABLE 1

| Cas9d variant | Amino acid alterations |
|---|---|
| C24 | N235NGSGAGGSY |
| C25 | S104A, F105H, V107S |
| C26 | P176S, S179A |
| C27 | E223A |
| C28 | T310A, P316G |
| C29 | P411A, Q413N, I414V |
| C30 | P411A, Q413N, I414V, R457K, N459S |
| C31 | Y430F, F432H |
| C32 | R457K, N459S |
| C33 | P475S, W476H |
| C34 | R557K |
| C35 | R653H |
| C36 | R951Q |

### Example 7. Identification of Amino Acid Alterations that Will Affect the Thermostability of the Cas9-gRNA-DNA Complex

Protein structure (PDB:5F9R) of Cas9 from S. pyogenes in complex with a guide RNA (gRNA) and a DNA fragment was analyzed to identify amino acid of key importance for the thermostability of this complex. Changes in these amino acids will lead to destabilizing domain-domain interactions, ion-sites, flexible regions eg. with high b-factor, salt-bridges, hydrogen bonds and hydrophobic areas in Cas9. Other amino acid changes will destabilize the interactions with the gRNA and the interactions with the DNA.

The changes could either be introduced individually or included as options in one or more libraries, when applying a selective screening system. Cas9 is a large molecule with more than 1300 amino acids and therefore sufficient destabilization for some biological systems might only be possible by introducing several substitutions simultaneously.

Amino acid alterations within different concepts can be found in Tables 2-4.

TABLE 2
Amino acid residues of importance for
Cas9 protein-protein interactions.

| Position | Suggested alterations |
|---|---|
| P176 | Substitution with A, G, S, T, or V |
| S179 | Substitution with A, G, T, V, or P |
| E223 | Substitution with Q, D, N, H, S, A, I, L, T, V, |
| | P, K, Y, W, F, G, C, or R |
| E260 | Substitution with Q or S |
| D261 | Substitution with N or S |
| T310 | Substitution with A or G |
| P316 | Substitution with G or D |
| P411 | Substitution with A or G |
| Q413 | Substitution with N or A |
| I414 | Substitution with V or A |
| Y430 | Substitution with F or V |
| F432 | Substitution with H or V |
| R457 | Substitution with K or S |
| N459 | Substitution with S |
| R461 | Substitution with K or S |
| R557 | Substitution with K or S |
| R653 | Substitution with H or N |
| E910 | Substitution with Q or S |
| L911 | Substitution with V or A |
| R951 | Substitution with Q or S |
| K1123 | Substitution with Q or S |
| W1126 | Substitution with Y or S |
| P1301 | Substitution with S or G |
| H1311 | Substitution with Y or S |
| P176 + S179 | P176S + S179A or P176G + S179N |
| E260 + D261 | E260Q + D261N or E260S + D261S |
| T310 + P316 | T310A + P316G or T310G + P316D |
| P411 + Q413 + | P411A + Q413N + I414V or P411G + |
| I414 | Q413A + I414A |
| Y430 + F432 | Y430F + F432H or Y430V + F432V |
| R457 + N459 | R457K + N459S |
| R457 + N459 + R461 | R457S + N459S + R461S |
| E910 + L911 | E910Q + L911V or E910S + L911A |
| K1123 + W1126 | K1123Q + W1126Y or K1123S + W1126S |
| P1301 + H1311 | P1301S + H1311Y or P1301G + H1311S |

TABLE 3
Amino acid residues of importance
for Cas9 protein-gRNA interactions.

| Position | Suggested alterations |
|---|---|
| S29 | Substitution with A, G, T, or V |
| K31 | Substitution with R, Q, H, M, or L |
| F32 | Substitution with Y, H, L, I, or M |
| L35 | Substitution with V, A, T, I, M, F, or H |
| K44 | Substitution with R, Q, H, M, or L |
| N46 | Substitution with D, S, T, or Q |
| E57 | Substitution with Q, D, N, or H |
| T62 | Substitution with S, A, V, or P |
| R63 | Substitution with K, H, Q, or E |
| K65 | Substitution with R, H, Q, M, or L |
| R66 | Substitution with K, H, Q, or E |
| R69 | Substitution with K, H, Q, or E |
| R70 | Substitution with K, H, Q, or E |
| R71 | Substitution with K, H, Q, or E |
| Y72 | Substitution with F, H, L, M, I, or W |
| R74 | Substitution with K, H, Q, or E |
| R75 | Substitution with K, H, Q, or E |
| R78 | Substitution with K, H, Q, or E |
| S104 | Substitution with A, T, or V |
| F105 | Substitution with H, L, M, I, Y, or W |
| V107 | Substitution with S, A, I, L, or T |
| E108 | Substitution with Q, D, or N |
| R115 | Substitution with K, H, Q, or E |
| H116 | Substitution with F, Y, Q, E, N, D, S, or T |
| V126 | Substitution with I, L, A, T, or S |
| H129 | Substitution with F, Y, Q, E, N, D, S, or T |
| Y136 | Substitution with F, H, L, M, I, or W |
| H160 | Substitution with F, Y, Q, E, N, D, S, or T |
| K163 | Substitution with R, H, Q, E, M , or L |
| F164 | Substitution with Y, H, M, L, I, or W |
| R165 | Substitution with K, H, Q, or E |
| Y325 | Substitution with F, H, L, M, I, or W |
| H328 | Substitution with F, Y, Q, E, N, D, S, or T |
| H329 | Substitution with F, Y, Q, E, N, D, S, or T |
| K336 | Substitution with R, H, M, or L |
| R340 | Substitution with K, H, Q, or E |
| Y347 | Substitution with F, H, M, L, or I |
| F351 | Substitution with H, Y, M, L, I, or W |
| F352 | Substitution with H, Y, M, L, I, or W |
| I363 | Substitution with F, L, M, V, A, or T |
| D364 | Substitution with E, Q, H, N, S, or T |
| R403 | Substitution with K, H, Q, or E |
| I448 | Substitution with L, M, V, or A |
| P454 | Substitution with A, G, S, T, or V |
| L455 | Substitution with I, V, M, T, N, F, or A |
| R457 | Substitution with K, H, Q, or E |
| N459 | Substitution with D, E, Q, H, S, T, or A |
| S460 | Substitution with A, G, T, or V |
| R461 | Substitution with K, H, Q, or E |
| F462 | Substitution with Y, H, L, I, V, or W |
| R467 | Substitution with K, H, Q, or E |
| T472 | Substitution with A, P, S, or V |
| Y515 | Substitution with F, H, M, or L |
| R661 | Substitution with K, H, Q, or E |
| S719 | Substitution with A, T, or V |
| L721 | Substitution with I, V, M, T, N, F, or A |
| H721 | Substitution with F, Y, Q, E, N, D, S, or T |
| K735 | Substitution with R, Q, H, M, or L |
| L738 | Substitution with I, V, M, T, N, F, or A |
| K742 | Substitution with R, Q, H, M, or L |
| M751 | Substitution with L, I, V, T, or K |
| S777 | Substitution with A |
| E779 | Substitution with Q, H, D, or N |
| D850 | Substitution with N, S, or A |
| K918 | Substitution with R, H, M, or L |
| Q933 | Substitution with E, H, K, or N |
| V982 | Substitution with A, T, L, or I |
| K1085 | Substitution with R, Q, H, M, or L |
| M1089 | Substitution with L, I, V, T, or K |
| P1090 | Substitution with A, G, S, T, or V |
| Q1091 | Substitution with E, H, K, or N |
| T1098 | Substitution with A, P, S, or V |
| E1099 | Substitution with Q, D, N, or H |
| V1100 | Substitution with A, G, T, S, I, or L |
| T1102 | Substitution with A, P, S, or V |
| G1103 | Substitution with A, P, or S |
| F1105 | Substitution with Y, H, L, I, V, or W |
| E1108 | Substitution with Q, D, N, or H |
| K1085 | Substitution with R, Q, H, M, or L |
| Q1091 | Substitution with E, N, D, or S |
| K1113 | Substitution with R, Q, H, M, or L |
| K1123 | Substitution with R, Q, H, M, or L |
| K1124 | Substitution with R, Q, H, M, or L |
| F1134 | Substitution with Y, H, L, I, V, or W |
| R1171 | Substitution with K, H, Q, or E |
| K1197 | Substitution with R, Q, H, M, or L |
| K1211 | Substitution with R, Q, H, M, or L |
| A1279 | Substitution with G, S,T, or V |
| R1279 | Substitution with K, H, Q, E, R, Q, H, M, or L |
| H1349 | Substitution with F, Y, Q, E, N, D, S, or T |
| Y1356 | Substitution with F, W, H, Q, E, N, D, S, or T |
| T1358 | Substitution with A, P, S, or |
| S104 + F105 + | S104A + F105H + V107S or S104G + |
| V107 | F105S + V107L |
| P475 + W476 | P475S + W476H or P475G + W476A |

TABLE 4
Amino acid residues of importance
for Cas9 protein-DNA interactions.

| Position | Suggested alterations |
|---|---|
| T13 | Substitution with S or A |
| N14 | Substitution with D, S, or A |
| S55 | Substitution with A |
| F916 | Substitution with H, L, M, I, or A |
| K918 | Substitution with Q, H, M, or L |
| Q920 | Substitution with E, N, S, or A |
| H698 | Substitution with F, Q, or N |
| Q695 | Substitution with E, N, or D |
| S777 | Substitution with A, N, or D |
| R765 | Substitution with K, H, or Q |
| R661 | Substitution with K, H, or Q |
| T924 | Substitution with S or A |
| R925 | Substitution with K, H, or Q |
| Q926 | Substitution with K, E, N, or D |
| K929 | Substitution with R, H, or Q |
| S960 | Substitution with A |
| S964 | Substitution with A |
| K968 | Substitution with Q, H, M, or L |
| N497 | Substitution with D, Q, E, or S |
| M495 | Substitution with K, Q, or L |
| F491 | Substitution with H or L |
| Y450 | Substitution with H, L, or F |
| T657 | Substitution with S, A, or N |
| D269 | Substitution with N, S, or A |
| G582 | Substitution with A, S, T, or V |
| Q894 | Substitution with E, N, or S |
| R976 | Substitution with K, Q, or H |
| Y1013 | Substitution with F or H |
| G1030 | Substitution with A, S, T, or V |
| A1032 | Substitution with G, S, T, or V |
| K1107 | Substitution with R, Q, or M |
| E1108 | Substitution with Q or D |
| S1109 | Substitution with A, T, N, or D |
| R1114 | Substitution with K, Q, or H |
| N1115 | Substitution with D, A, or S |
| S1116 | Substitution with A |
| D1117 | Substitution with N, S, or A |
| K1118 | Substitution with Q, H, M, or L |
| D1135 | Substitution with N, S, or A |
| E1219 | Substitution with Q, D, or N |
| S1216 | Substitution with A |
| E1243 | Substitution with Q, D, N, S, or A |
| D1284 | Substitution with N, S, or A |
| R1333 | Substitution with K, Q, or H |
| K1334 | Substitution with R, Q, M, or L |
| R1335 | Substitution with K, Q, or H |
| T1337 | Substitution with A or S |
| S1338 | Substitution with A |

### Example 8. Screening of the cas9d Library by GFP Fluorescence in B. subtilis

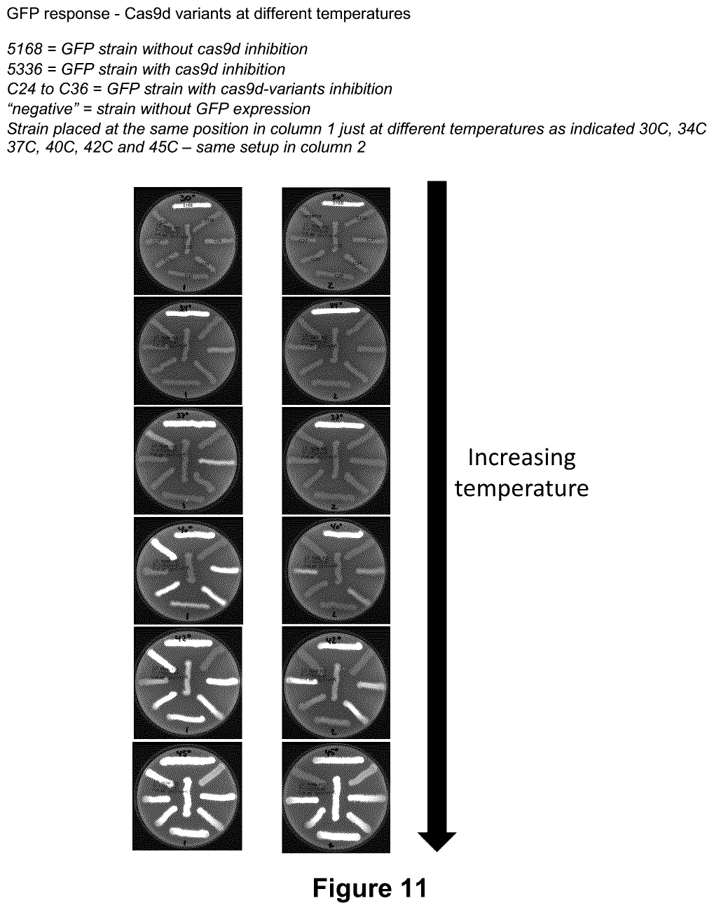

The library of different site-directed Cas9d variants listed in Table 1 were transformed into PP5168 and plated onto LBPG+6 ug/m of chloramphenicol to select for integration at the chromosomal pel locus as described in Example 2. The plates were incubated at 34° C. overnight and all clones were tested for green fluorescence. None of the correctly cloned variants show fluorescence and is equal to the wild type cas9d control strain PP5336. Individual transformants of the different cas9d variants were re-streaked on fresh plates LBPG+6 ug/ml of chloramphenicol and incubated at 30° C., 34 C, 37° C., 40° C., 42° C., 45° C. overnight. The next day the different variant clones were tested for fluorescence at the different temperatures. FIG. 11 shows an example of the plates and their GFP fluorescence. A table listing the qualitative data of the different variants can be seen in Table 5, where the level of fluorescence is scored from zero to four. The zero score corresponds to no fluorescence and the score of four is highest fluorescence.

TABLE 5
Qualitative GFP screening of Cas9d variants.

| Cas9d variant | 30° C. | 34° C. | 37° C. | 40° C. | 42° C. | 45° C. |
|---|---|---|---|---|---|---|
| No Cas9d | 4 | 4 | 4 | 4 | 4 | 4 |
| (PP5168) | | | | | | |
| wt Cas9d | 0 | 0 | 0 | 0 | 0 | 2 |
| (PP5336) | | | | | | |
| C24 | 0 | 0 | 2 | 4 | 4 | 4 |
| C25 | 0 | 1 | 3 | 4 | 4 | 4 |
| C26 | 0 | 0 | 0 | 0 | 3 | 4 |
| C27 | 0 | 0 | 0 | 0 | 3 | 4 |
| C28 | 0 | 0 | 0 | 4 | 4 | 4 |
| C29 | 0 | 0 | 0 | 4 | 4 | 4 |
| C30 | 0 | 0 | 0 | 3 | 4 | 4 |
| C31 | 0 | 0 | 0 | 1 | 3 | 4 |
| C32 | 0 | 0 | 0 | 0 | 2 | 4 |
| C33 | 0 | 0 | 0 | 2 | 4 | 4 |
| C34 | 0 | 0 | 0 | 1 | 4 | 4 |
| C35 | 0 | 0 | 0 | 0 | 1 | 4 |
| C36 | 0 | 0 | 0 | 0 | 1 | 4 |

As a positive control the strain PP5168 show full fluorescence on all plates with a score of four. The PP5336 strain with the Cas9d protein show full repression of GFP fluorescence up to 42° C. and a slight de-repression at 45° C. with a fluorescence score of two. The two Cas9d variants C24 and C25 show de-repression already at 37° C. and 34° C. respectively. The Cas9d variants C28, C29, C30, C31, C33, C34 start to show de-repression at 40° C. and the variants C26, C27, C32; C35 and C36 start to show de-repression at 42° C. Even between variants in the same category differences in fluorescence can be seen. The two variants C28 and C29 go from zero fluorescence at 37° C. to a full score of four at 40° C. C while the variant C31 has to reach 45° C. to show a full score of four. All of the Cas9d variants in the table show a pattern of de-repression as a response to temperature which is very different from the original Cas9d sequence. This will allow a specific design of Cas9d that matches the organism and process for which the CRISPRi inhibition and induction is going to take place.

### Example 9. Testing the Cas9d Variants in Liquid Cultures

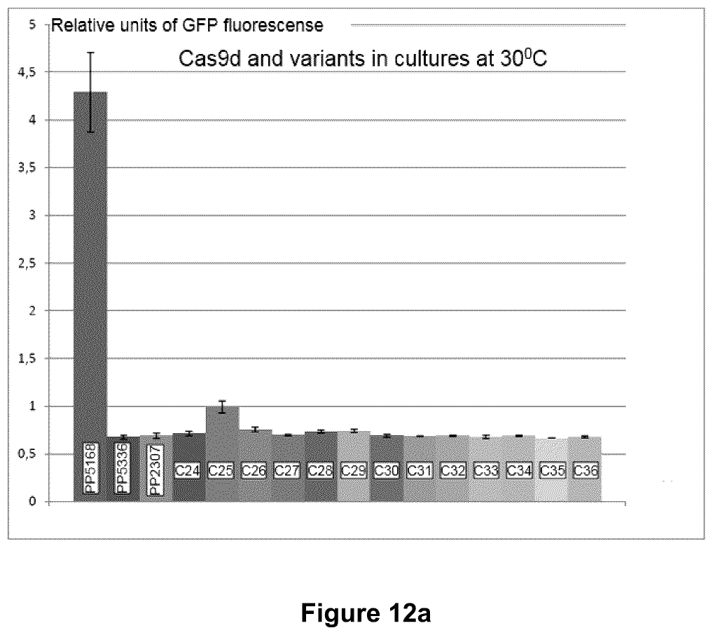

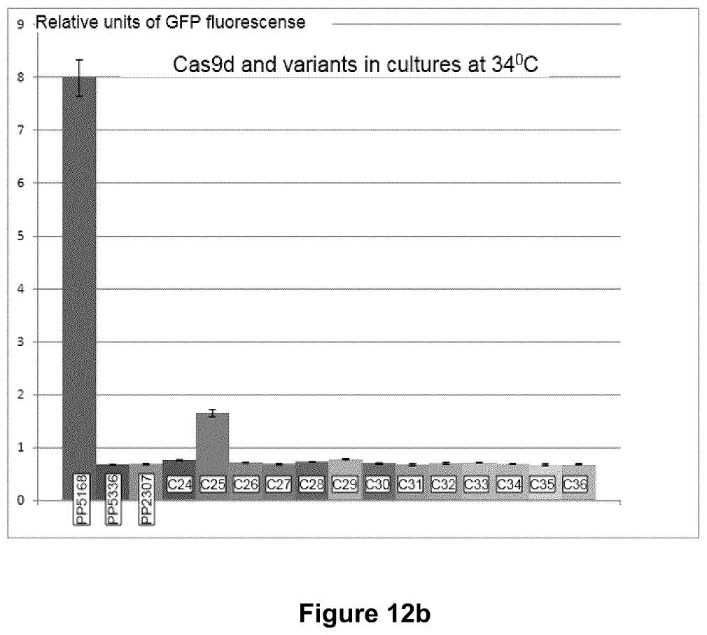

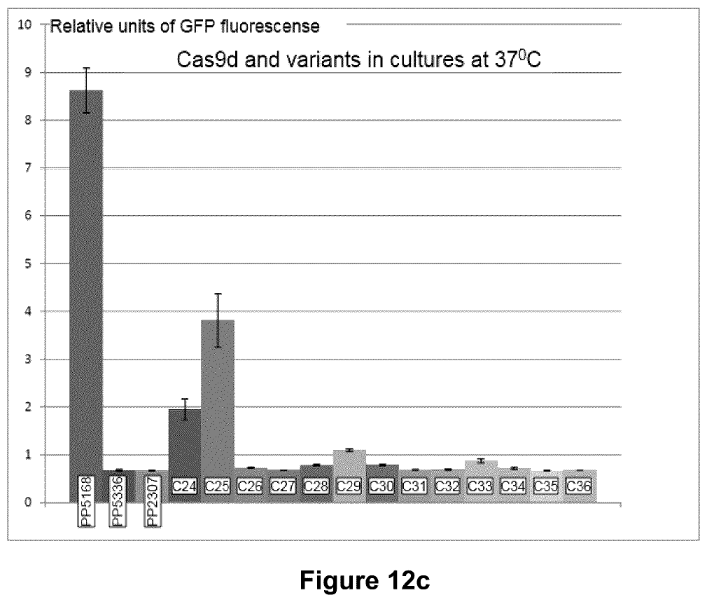

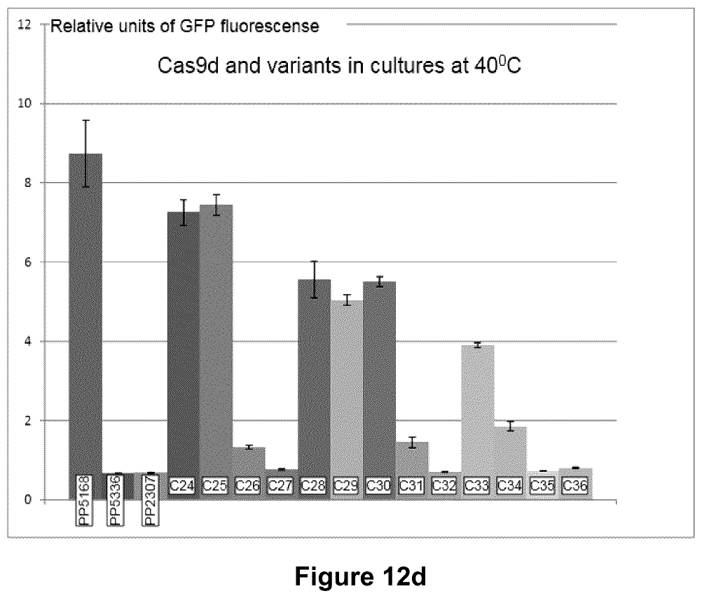

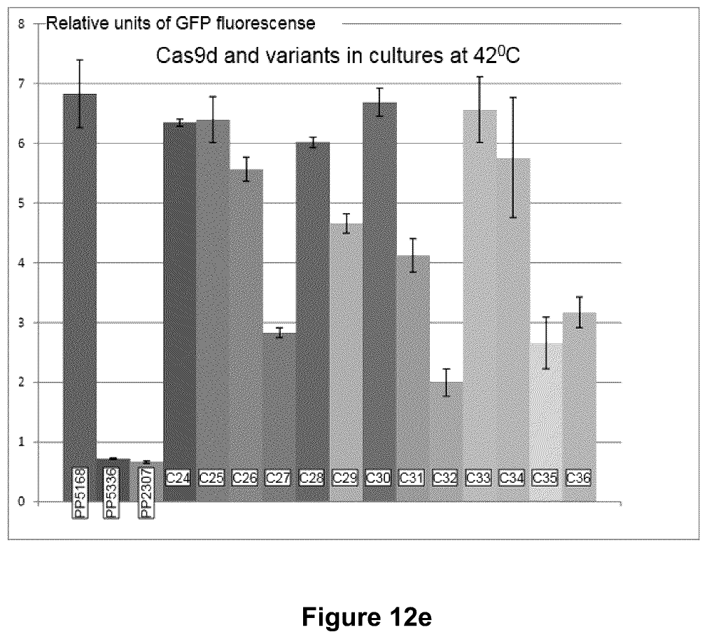

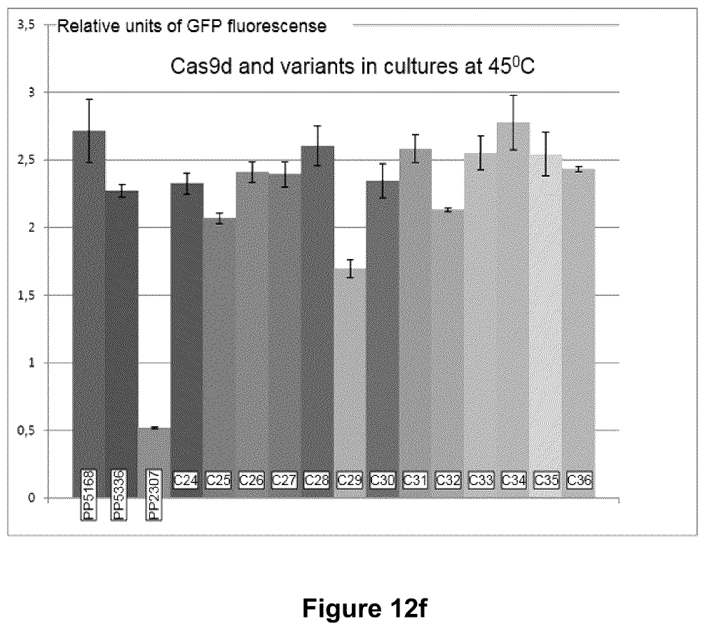

The B. subtilis strains screened for Cas9d variants in Example 8 were also tested in liquid cultures to determine if a response to temperature can be observed as demonstrated on solid media on plates. Three control strains PP5168, PP5336 and PP2307 and the individual 13 variant clones were first inoculated in fresh TY media and incubated at six individual temperatures ranging from 30° C. to 45° C. for 18 hours. The strain PP2307 is a blind control where no GFP gene is present. The positive control strain PP5168 show fluorescence at all temperatures as expected. This strain has no Cas9d gene integrated at the chromosome and cannot form an inhibitory CRISPRi complex to silence the GFP gene. Consequently the strain expresses GFP regardless of the growth and temperature. The PP5336 strain with the wt cas9d gene can form the CRISPRi complex and inhibit expression of the GFP fluorescent protein at all temperatures (FIGS. 12a and 12b), except at 45° C. as seen in FIG. 12f. This is in good agreement with the results from Example 7 where the same two strains PP5168 and PP5336 were tested on solid agar medium. The conclusions from the results on the PP5336 strain is that the wt Cas9d protein can form a stable CRISPRi complex at temperatures up to 42° C. but at 45° C. the complex is not functional and cannot inhibit the expression of GFP. The 13 Cas9d variants grown in liquid medium also show a similar pattern to what was observed on agar plates. The most temperature sensitive variant on plates C25 is also de-repressed in this experiment and is already showing elevated fluorescence at 30° C. The variant C24 start to show elevated fluorescence at 37° C. Both of these variants show strong de-repression at 40° C. and above. The variants C28, C29, C30 and C33 show de-repression between 37° C. and 40° C., and the variants C26 and C31 show strong de-repression at 42° C. The variants C27, C32, C35 and C36 show modest de-repression at 42° C. and full de-repression at 45° C.

The conclusion of this experiment is that the 13 site-directed Cas9d variants all show stability properties which are very different from the original Cas9d. All variants show to a varying degree an increased temperature sensitivity both on solid medium and in fluent cultures.

This finding show that it is possible to engineer the Cas9d protein in way that changes the temperature stability of the Cas9d-gRNA inhibitor complex. The changed temperature range can be employed to control the availability of Cas9d-gRNA complex by a switch in temperature which suits the host organism of choice and the physiological conditions of the preferred growth parameters.

### Example 10. Testing the cas9d Variants in Fluent Cultures—Temperature Shift

In this experiment the three control strains PP5168, PP5336 and PP2307 and the individual 13 variant clones were first inoculated in fresh TY media and incubated for 18 hours at 30° C. The cultures were then divided in two and incubated at 42° C. and 45° C. After 18 hours, the cultures were diluted and analysed for their GFP fluorescence. The results are shown in FIG. 13a. The objective is to determine if a sudden change in temperature can destabilize the inhibitor complex once it is bound to the target DNA. The results in FIG. 13 show that the variants C25, C26, C30 and C33 reacts very efficiently to the temperature change and increase the level of GFP as a result of destabilized inhibitor complex and de-repression of the GFP gene.

This finding clearly show that it is possible to engineer the Cas9d protein which changes the temperature stability of the Cas9d-gRNA inhibitor complex when already bound to its target DNA. The changed temperature range can be employed to control the availability of Cas9d-gRNA complex by a switch in temperature which suits the host organism of choice and the physiological conditions of the preferred growth parameters. By choosing the right Cas9d variant and combine it with a suitable temperature profile it is possible to control the efficacy of the Cas9d-gRNA complex and change the affinity to a DNA target of choice. In this way it is possible to regulated any target gene by a simple change of temperature.

### Example 11. Construction of B. licheniformis Strains with Temperature Sensitive Cas9d Variants C25, C30 and C33

A subset of the best site-directed variants of Cas9d described in example 6 for B. subtilis (C25, C30 and C33) were transferred to B. licheniformis strain PP5007 to study the inhibition profile in this organism. The three different variants of Cas9d were each cloned by SOE-PCR on a plasmid. The resulting plasmids were each introduced to B. licheniformis PP5007 by conjugation. Chromosomal insertion of the expression cassette consisting of the cas9d gene expressed from the forD promoter, the gDNA(P4199) transcribed from the PamyQsc promoter and the cat gene conferring chloramphenicol resistance was obtained by homologous recombination, resulting in chromosomal structure as illustrated in FIG. 5. The gDNA(P4199) is transcribed into gRNA(P4199) which directs Cas9d to the P4199 promoter and inhibit transcription. All four amyL gene copies are expressed from the P4199 promoter and the Cas9d-gRNA complex can potentially bind and inhibit alpha-amylase expression through strong interaction to the P4199 target, as illustrated in FIG. 6.

The final B. licheniformis strains were named as follows:
PP5541—Cas9d variant C25
PP5360—Cas9d variant C30
PP5542—Cas9d variant C33

All three strains are identical to PP5021 described in Example 3 except for the amino acid substitutions in Cas9d described in table 1. The Cas9d variants were screened as temperature sensitive variants in B. subtilis.

### Example 12. Alpha-Amylase Expression in B. licheniformis PP5541, PP5360 and PP5542—Temperature Shift

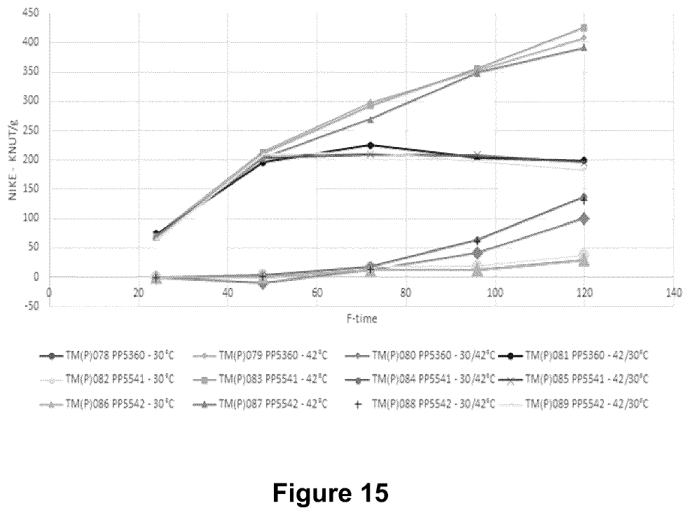

The B. licheniformis strains described in Example 11 were tested with respect to alpha-amylase productivity at different temperatures in fed-batch cultivations as described above. Full amylase productivity was observed when the strains were grown at 42° C. all five days (FIG. 15) showing full de-repression of the Cas9d inhibition complex for all three variants. In another set of experiments the strains were grown for two days either at 30° C. or 42° C. Then the temperatures were shifted to 42° C. and 30° C., respectively, to observe the effect on amylase expression from the P4199 promoter. Culture samples were taken each day to measure the activity of amylase. The results are presented in FIG. 15, which shows a clear effect on the temperature shifts at 48 hours. When the strains were grown at 30° C. for the first two days, the presence of the Cas9d variants with the gRNA(P4199) resulted in repression of amylase productivity. When the temperature was raised to 42° C., the productivity of amylase was increased for all three strains with PP5541(C25) being the most responsive to the elevated temperature. These data show that the inhibition of transcription from the P4199 promoter is de-repressed when the temperature is raised and that the variant C25 is the most temperature sensitive. This is in good agreement with what is observed on plates with the identical Cas9d variants in B. subtilis.

When starting the cultivation at 42° C., good amylase productivity was observed for all the tested strains (FIG. 15). When the temperature was lowered to 30° C. after two days, the productivity of amylase was clearly reduced, indicating a repression of transcription from the P4199 promoter. In conclusion, the cultivated B. licheniformis strains confirmed the temperature sensitivity of the Cas9d variants within an applicable temperature interval to be used for B. licheniformis.

### Example 13. Alpha-Amylase Expression Profile of the Cas9d Variant C25 in B. licheniformis

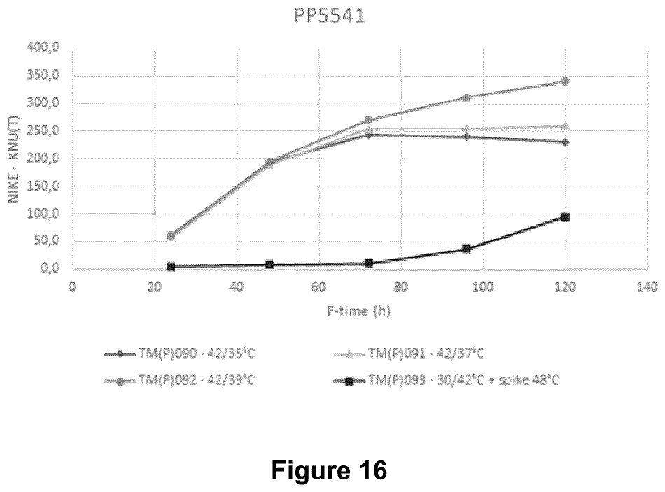

The B. licheniformis strains PP5541 in Example 11 and 12 were further tested with respect to alpha-amylase productivity and response to a temperature shift in fed-batch cultivation. Full amylase productivity was observed when the strain was grown at 42° C. for the first two days in three separate fermentations (FIG. 16). The temperature was then shifted down to either 35° C., 37° C. or 39° C. in each of the three fermentations. In FIG. 16 is shown that the downshift to 35° C. and 37° C. efficiently repress the expression of the amylase, while the downshift to 39° C. still allow some expression.

### Example 14. Library of Cas9d Variants in B. subtilis Based on Temperature-Sensitive Variant C25

To obtain Cas9d variants with increased temperature sensitivity, a library of expression cassettes based on Cas9d variant C25 was constructed as described in Example 5 and inserted in the chromosome at the pel locus. The different site-directed variants of the Cas9d protein were, in addition to the amino acid substitution S104A, F105H, V107S found in C25, based on the Cas9 structure (PDB: 5F9R) described in Example 7. The site-directed variants were introduced by SOE-PCR and cloned as DNA fragments with flanking DNA identity to the pel locus in B. subtilis as earlier described in WO2006042548. The library of different variants was introduced by transformation into competent PP5168 as described in Example 5. The final map of the pel locus is illustrated in FIG. 9 with the only difference being codon modifications to fit the different amino acid changes in Cas9d. An initial screen for identification of improved temperature sensitive Cas9d variants was performed on LBPGS +6 ug/ml of chloramphenicol plates and incubation overnight at 30° C., 34° C., 37° C. and 42° C. Next day, colonies with green coloring at 34° C., 37° C. and 42° C., which appeared to be more thermosensitive than 025, were identified and isolated. The amino acid substitutions in the selected strains were determined by sequencing. The resulting site-directed variants in Cas9d is presented in Table 6.

TABLE 6

| Cas9d clones | Amino acid alterations |
|---|---|
| C37 | S104A, F105H, V107S, P176S, S179A, E223S, |
| | E260Q, D261N, R653H, Q739S, H1311Y |
| C38 | S104A, F105H,V107S, E260Q, D261N, T310A, |
| | P316G, Y430F, F432H, Q739S, H1311Y |
| C39 | S104A, F105H, V107S, T310A, P316G, Q739S, |
| | R951Q, |
| C40 | S104A, F105H, V107S, E260Q, D261N, Y430F, |
| | F432H, Q739S |
| C41 | S104A, F105H, V107S, Q739S, R951Q, H1311Y |
| C42 | S104A, F105H, V107S, P411A, Q413N, I414V |
| C43 | S104A, F105H, V107S, P411A, Q413N, I414V, |
| | R457K, N459S |

### Example 15. Screening of the Improved Temperature Sensitive Cas9d Strains by GFP Fluorescence in B. subtilis

The identified strains with different site-directed Cas9d variants listed in Table 6 were streaked onto LBPG+6 ug/mi of chloramphenicol plates and incubated at 30° C., 34° C., 37° C., and 42° C. overnight. The next day the different variant clones were tested for fluorescence at the different temperatures as shown in FIG. 11. A table listing the qualitative data of the different variants can be seen in Table 7, where the level of fluorescence is scored from zero to four. The zero score corresponds to no fluorescence and the score of four is highest fluorescence.

TABLE 7
Qualitative GFP screening of improved
temperature sensitive Cas9d variants.

| Cas9d variant | 30° C. | 34° C. | 37° C. | 42° C. |
|---|---|---|---|---|
| No Cas9d (PP5168) | 4 | 4 | 4 | 4 |
| wt Cas9d (PP5200) | 0 | 0 | 0 | 0 |
| C25 | 0 | 0 | 2 | 4 |
| C37 | 0 | 1 | 4 | 4 |
| C38 | 1 | 3 | 4 | 4 |
| C39 | 0 | 1 | 4 | 4 |
| C40 | 0 | 1 | 4 | 4 |
| C41 | 0 | 0 | 4 | 4 |
| C42 | 0 | 2 | 4 | 4 |
| C43 | 1 | 2 | 4 | 4 |

As a positive control strain PP5168 shows full fluorescence on all plates with a score of four. Strain PP5200 with the Cas9d protein shows full repression of GFP fluorescence up to 42° C. The Cas9d variants C38 and C43 show slight de-repression already at 30° C. The Cas9d variants C37, C39, C40, and C42 show beginning de-repression at 34° C., whereas C41 show de-repression at 37° C. All the Cas9d variants constructed on basis of C25 showed increased temperature sensitivity compared to C25, with a score of 4 at 37° C.

### Example 16. Testing the Improved Cas9d Variants in Liquid Cultures

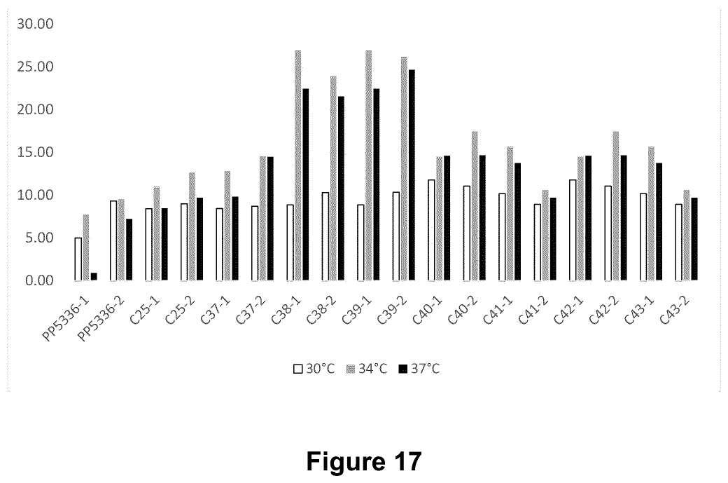

The B. subtilis strains with Cas9d variants described in Example 14 were also tested in liquid cultures to determine the response to temperature. The strains PP5336, expressing the wt Cas9d, C25 expressing a temperature sensitive variant of Cas9d, and the individual seven variants were first inoculated in fresh TY media and incubated at 30° C., 34° C., 37° C., and 42° C. for 18 hours. Two independent cultures were inoculated for each strain. The seven improved Cas9d variants grown in liquid medium show a similar pattern to what was observed on agar plates (FIG. 17). At 42° C. all strains showed significant high GFP activities, and data is for illustrative reasons not included in FIG. 17. The most temperature sensitive variant C38 identified on plates is also de-repressed in this experiment and is already showing high fluorescence at 34° C.

The conclusion of this experiment is that the improved seven site-directed Cas9d variants all show stability properties which are different from Cas9d C25 variant. All variants show to a varying degree an increased temperature sensitivity both on solid medium and in fluent cultures.

### Example 17. Library of Additional Cas9d Variants in B. subtilis

To obtain additional temperature sensitive variants of Cas9d, with amino acid substitutions that could also be de-stabilizing of Cas9d similar to the amino acid substitutions described in Example 6, a library of expression cassettes was constructed as the one described in Example 6 and inserted at the pel locus. The different site-directed variants of the Cas9d protein were based on the Cas9 structure (PDB: 5F9R) further described in Example 7. The site-directed variants were introduced by SOE-PCR and cloned as DNA fragments with flanking DNA identity to the pel locus in B. subtilis as earlier described in WO2006042548. The library of different variants was introduced by transformation into competent PP5168 as described in Example 5. The final map of the pel locus is as already shown in FIG. 9 with the only difference being the different amino acid changes in Cas9d. An initial screen for identification of improved temperature sensitive Cas9d variants was performed on LBPGS plates and incubation overnight at 30° C. and 42° C. Next day, colonies with green coloring at 42° C. were identified and isolated. The resulting site-directed variants in Cas9d is presented in Table 8.

TABLE 8

| Cas9d variant | Amino acid alterations |
|---|---|
| C44 | T310G, P316D |
| C45 | P411G, Q413A, I414A |
| C46 | Y430V, F432V |
| C47 | E910S, L911A |
| C48 | K1123S, W1126S |

### Example 18. Screening of New Temperature Sensitive Cas9d Strains by GFP Fluorescence in B. subtilis

The identified strains with different site-directed Cas9d variants listed in Table 8 were streaked onto LBPG+6 ug/ml of chloramphenicol and incubated at 30° C., 34° C., 37° C., and 42° C. overnight. The next day the different variant clones were tested for fluorescence at the different temperatures similarly to the method illustrated in FIG. 11. A table listing the qualitative data of the different variants can be seen in Table 9, where the level of fluorescence is scored from zero to four. The zero score corresponds to no fluorescence and the score of four is highest fluorescence.

TABLE 9
Qualitative GFP screening of improved
temperature sensitive Cas9d variants.

| Cas9d variant | 30° C. | 34° C. | 37° C. | 42° C. |
|---|---|---|---|---|
| No Cas9d (PP5168) | 4 | 4 | 4 | 4 |
| wt Cas9d (PP5200) | 0 | 0 | 0 | 0 |
| C44 | 1 | 3 | 4 | 4 |
| C45 | 0 | 0 | 1 | 4 |
| C46 | 0 | 2 | 4 | 4 |
| C47 | 0 | 1 | 4 | 4 |
| C48 | 0 | 1 | 4 | 4 |

As a positive control the strain PP5168 show full fluorescence on all plates with a score of four. The PP5200 strain with the wt Cas9d protein show full repression of GFP fluorescence up to 42° C. The Cas9d variant C44 show slight de-repression already at 30° C., the Cas9d variants C46, C47, and C48 show de-repression at 34° C., and C45 show de-repression at 37° C.

### Example 19. Construction of B. licheniformis Strains with Temperature Sensitive Cas9d Variants C38, C40, C42 and C43

Four of the temperature sensitive Cas9d variants (C38, C40, C42, and C43) were selected for plasmid construction to be used for chromosomal integration in forD locus of B. licheniformis. The B. licheniformis PP5007 strain, described in Example 2, was used as parental strain for insertion of the temperature sensitive cas9d variants and a gDNA(P4199) expressed from the PamyQsc promoter. Plasmids were constructed similarly to what is described in Example 2, with the selected Cas9 variants. The resulting plasmids were then introduced to B. licheniformis PP5007 by conjugation. Chromosomal insertion of the expression cassette consisting of the cas9d gene expressed from the forD promoter, the gDNA(P4199) transcribed from the PamyQsc promoter and the cat gene conferring chloramphenicol resistance was obtained by homologous recombination, resulting in chromosomal structure as illustrated in FIG. 5. The constructed strains are shown in Table 10. The gDNA(P4199) is transcribed into gRNA(P4199) which directs Cas9d to the P4199 promoter and inhibit transcription. All four amyL gene copies in strain BKQ3746, BKQ3748, BKQ3750, and BKQ3752 are expressed from the P4199 promoter and the Cas9d-gRNA complex can potentially bind and inhibit alpha-amylase expression through strong interaction to the P4199 target, as illustrated in FIG. 6.

TABLE 10
B. licheniformis strains containing temperature
sensitive cas9d variants, gDNA(P4199), and four copies
of an amylase expressed from the P4199 promoter.

| B. licheniformis | Cas9d | |
|---|---|---|
| strain | variant | Amino acid substitutions |
| BKQ3746 | C38 | S104A, F105H, V107S, E260Q, |
| | | D261N, T310A, P316G, Y430F, |
| | | F432H, Q739S, H1311Y |
| BKQ3748 | C40 | S104A, F105H, V107S, E260Q, |
| | | D261N, Y430F, F432H, Q739S |
| BKQ3750 | C42 | S104A, F105H, V107S, P411A, |
| | | Q413N, 1414V |
| BKQ3752 | C43 | S104A, F105H, V107S, P411A, |
| | | Q413N, 1414V, R457K, N459S |

### Example 20. Alpha-Amylase Expression in B. licheniformis BKQ3746, BKQ3748, BKQ3750, and BKQ3752—Temperature Shift

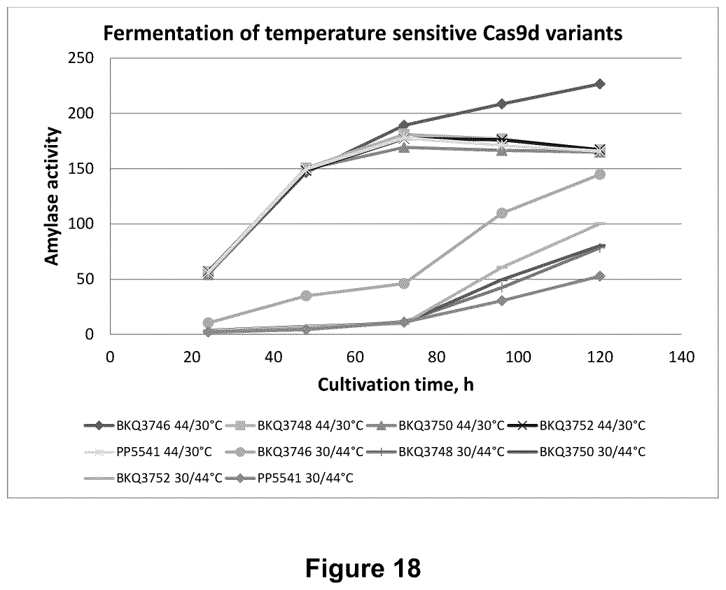

The B. licheniformis strains described in Example 18 were tested with respect to alpha-amylase productivity upon temperature shifts in fed-batch cultivations as described above. The strains were grown for two days either at 30° C. or 44° C. Then the temperatures were shifted to 44° C. and 30° C., respectively, to observe the effect on amylase expression from the P4199 promoter. Culture samples were taken each day to measure the activity of amylase. The results are presented in FIG. 18, which show a clear effect on the temperature shifts. When the strains were grown at 30° C. for the first two days, the presence of the Cas9d variants with the gRNA(P4199) resulted in inhibition of amylase productivity, although BKQ3746 expressing Cas9d variant C38, was found to be leaky. When the temperature was raised to 44° C., the productivity of amylase was increased, with BKQ3746(C38) >BKQ3752(C43) >BKQ3750(C42) >BKQ3748(C40) >PP5541(C25). These data show that the inhibition of transcription from the P4199 promoter is de-repressed when the temperature is raised. This is in good agreement with what is observed on plates with the identical Cas9d variants in B. subtilis. When starting the cultivation at 44° C., good amylase productivity was observed for all the tested strains (FIG. 18). When the temperature was lowered to 30° C. after two days, the productivity of amylase was clearly reduced, indicating a repression of transcription from the P4199 promoter.

In conclusion, the cultivated B. licheniformis strains confirmed the temperature sensitivity of the used Cas9d variants within an applicable temperature interval to be used for B. licheniformis.

### PREFERRED EMBODIMENTS

1) A temperature-sensitive variant of a Class-II Cas9 protein (tsCas9), said variant comprising at least one alteration of one or more amino acid important for protein stability or for stability of a complex formed between the Class-II Cas9 protein, one or more guide-RNA (gRNA), and one or more corresponding genome target sequence, wherein the at least one alteration is a substitution, insertion, or deletion of 1-10 amino acids; preferably the at least one alteration is a substitution or deletion of 1-10 amino acids; most preferably the at least one alteration is a substitution.

2) The tsCas9 according the first embodiment, said variant being a nickase or nuclease-null variant; preferably, said tsCas9 comprising an alteration of an amino acid corresponding to position 10 and/or position 840 of SEQ ID NO:2; more preferably said variant comprises a substitution of aspartic acid for alanine, D10A, and/or a substitution of histidine for alanine, H840A.

3) The tsCas9 according to any of embodiments 1-2, wherein the at least one alteration is in a position corresponding to a position selected from the group consisting of T13, N14, S29, K31, F32, L35, K44, N46, S55, E57, T62, R63, K65, R66, R69, R70, R71, Y72, R74, R75, R78, S104, F105, V107, E108, R115, H116, V126, H129, Y136, H160, K163, F164, R165, P176, S179, E223, N235, E260, D261, D269, T310, P316, Y325, H328, H329, K336, R340, Y347, F351, F352, I363, D364, R403, P411, Q413, I414, Y430, F432, I448, Y450, P454, L455, R457, N459, S460, R461, F462, R467, T472, F491, M495 N497, Y515, R557, G582, R653, T657, R661, Q695, H698, S719, L720, H721, K735, L738, K742, M751, R765 S777, E779, D850, Q894, E910, L911, F916, K918, Q920, T924, R925, Q926, K929, Q933, R951, S960, S964, K968, R976, V982, Y1013, G1030, A1032, K1085, M1089, P1090, Q1091, T1098, E1099, V1100, T1102, G1103, F1105, K1107 E1108, S1109, K1113, R11414, N1115, S1116, D1117, K1118, K1123, K1124, F1134, D1135, W1126, K1135, R1171, K1197, K1211, S1216, E1219, E1243, R1279, D1284, P1301, H1311, R1333, K1334, R1335, T1337, S1338, H1349, Y1356, and T1358 of SEQ ID NO:2.

4) The tsCas9 according to any of embodiments 1-3, wherein the at least one amino acid alteration is in a position corresponding to a position selected from the group consisting of P176, S179, E223, E260, D261, T310, P316, P411, Q413, I414, Y430, F432, R457, N459, R461, R557, R653, E910, L911, R951, K1123, W1126, P1301, and H1311 of SEQ ID NO:2.

5) The tsCas9 according to any of embodiments 1-3, wherein the at least one amino acid alteration is in a position corresponding to a position selected from the group consisting of S29, K31, F32, L35, K44, N46, E57, T62, R63, K65, R66, R69, R70, R71, Y72, R74, R75, R78, S104, F105, V107, E108, R115, H116, V126, H129, Y136, H160, K163, F164, R165, Y325, H328, H329, K336, R340, Y347, F351, F352, I363, D364, R403, I448, P454, L455, R457, N459, S460, R461, F462, R467, T472, Y515, R661, S719, L720, H721, K735, L738, K742, M751, S777, E779, D850, K918, Q933, V982, K1085, M1089, P1090, Q1091, T1098, E1099, V1100, T1102, G1103, F1105, E1108, K1113, K1123, K1124, F1134, R1171, K1197, K1211, R1279, H1349, Y1356, and T1358 of SEQ ID NO:2.

6) The tsCas9 according to any of embodiments 1-3, wherein the at least one amino acid alteration is in a position corresponding to a position selected from the group consisting of T13, N14, S55, D269, Y450, F491, M495, N497, G582, T657, R661, Q695, H698, R765, S777, Q894, F916, K918, Q920, T924, R925, Q926, K929, S960, S964, K968, R976, Y1013, G1030, A1032, K1107, E1108, S1109, R1114, N1115, S1116, D1117, K1118, D1135, S1216, E1219, E1243, D1284, R1333, K1334, R1335, T1337, and S1338 of SEQ ID NO:2.

7) The tsCas9 according to any of embodiments 1-3, wherein the at least one amino acid alteration is in a position corresponding to a position selected from the group consisting of S29, K31, F32, L35, K44, N46, E57, T62, R63, K65, R66, R69, R70, R71, Y72, R74, R75, R78, S104, F105, V107, E108, R115, H116, V126, H129, Y136, H160, K163, F164, R165, P176, S179, E223, E260, D261, T310, P316, Y325, H328, H329, K336, R340, Y347, F351, F352, I363, D364, R403, P411, Q413, I414, Y430, F432, I448, P454, L455, R457, N459, S460, R461, F462, R467, T472, Y515, R557, R653, R661, S719, L720, H721, K735, L738, K742, M751, S777, E779, D850, E910, L911, K918, Q933, R951, V982, K1085, M1089, P1090, Q1091, T1098, E1099, V1100, T1102, G1103, F1105, E1108, K1113, K1123, K1124, F1134, W1126, R1171, K1197, K1211, R1279, P1301, H1311, H1349, Y1356, and T1358 of SEQ ID NO:2.

8) The tsCas9 according to any of embodiments 1-3, wherein the at least one amino acid alteration is in a position corresponding to a position selected from the group consisting of T13, N14, S55, P176, S179, E223, E260, D261, D269, T310, P316, P411, Q413, I414, Y430, F432, Y450, R457, N459, R461, F491, M495, N497, R557, G582, R653, T657, R661, Q695, H698, R765, S777, Q894, E910, L911, F916, K918, Q920, T924, R925, Q926, K929, R951, S960, S964, K968, R976, Y1013, G1030, A1032, K1107, E1108, S1109, R1114, N1115, S1116, D1117, K1118, K1123, W1126, D1135, S1216, E1219, E1243, D1284, P1301, H1311, R1333, K1334, R1335, T1337, and S1338 of SEQ ID NO:2.

9) The tsCas9 according to any of embodiments 1-3, wherein the at least one amino acid alteration is in a position corresponding to a position selected from the group consisting of T13, N14, S29, K31, F32, L35, K44, N46, S55, E57, T62, R63, K65, R66, R69, R70, R71, Y72, R74, R75, R78, S104, F105, V107, E108, R115, H116, V126, H129, Y136, H160, K163, F164, R165, D269, Y325, H328, H329, K336, R340, Y347, F351, F352, I363, D364, R403, I448, Y450, P454, L455, R457, N459, S460, R461, F462, R467, T472, F491, M495, N497, Y515, G582, T657, R661, Q695, H698, S719, L720, H721, K735, L738, K742, M751, R765, S777, E779, D850, Q894, F916, K918, Q920, T924, R925, Q926, K929, Q933, S960, S964, K968, R976, V982, Y1013, G1030, A1032, K1085, M1089, P1090, Q1091, T1098, E1099, V1100, T1102, G1103, F1105, K1107, E1108, S1109, K1113, R1114, N1115, S1116, D1117, K1118, K1123, K1124, F1134, D1135, R1171, K1197, K1211, S1216, E1219, E1243, R1279, D1284, R1333, K1334, R1335, T1337, S1338, H1349, Y1356, and T1358 of SEQ ID NO:2.

10) The tsCas9 according to any of embodiments 1-3, wherein the at least one alteration is in a position corresponding to a position selected from the group consisting of S104, F105, V107, P176, S179, E223, N235, T310, P316, P411, Q413, I414, Y430, F432, R457, N459, P475, W476, R557, R653, and R951 of SEQ ID NO:2.

11) The tsCas9 according to any of embodiments 1-3, wherein the at least one amino acid alteration is selected from the group consisting of T13S, T13A, N14D, N14S, N14A, S29A, S29G, S29T, S29V, K31R, K31Q, K31H, K31M, K31L, F32Y, F32H, F32H, F32L, F32I, F32M, L35V, L35A, L35T, L35I, L35M, L35F, L35H, K44R, K44Q, K44H, K44M, K44L, K44D, K44N, K44H, K44M, K44L, N46D, N46S, N46T, N46Q, S55A, E57Q, E57D, E57N, E57H, T62S, T62A, T62V, T62P, R63K, R63H, R63Q, R63E, K65R, K65H, K65Q, K65M, K65L, R66K, R66H, R66Q, R66E, R69K, R69H, R69Q, R69E, R70K, R70H, R70Q, R70E, R71K, R71H, R71Q, R71E, Y72F, Y72H, Y72L, Y72M, Y72I, Y72W, R74K, R74H, R74Q, R74E, R75K, R75H, R75Q, R75E, R78K, R78H, R78Q, R78E, S104A, S104A, S104T, S104V, F105H, F105L, F105M, F105I, F105Y, F105W, V107S, V107A, V107I, V107L, V107T, E108Q, E108D, E108N, R115K, R115H, R115Q, R115E, H116F, H116Y, H116Q, H116E, H116N, H116D, H116S, H116T, V126I, V126L, V126A, V126T, V126S, H129F, H129Y, H129Q, H129E, H129N, H129D, H129S, H129T, Y136F, Y136H, Y136L, Y136M, Y136I, Y136W, H160F, H160Y, H160Q, H160E, H160N, H160D, H160S, H160T, K163R, K163H, K163Q, K163E, K163M, K163L, F164Y, F164H, F164M, F164L, F164I, F164W, R165K, R165H, R165Q, R165E, P176A, P176G, P176S, P176T, P176V, S179A, S179G, S179T, S179V, S179P, E223Q, E223D, E223N, E223H, E223S, E223A, E223I, E223L, E223T, E223V, E223P, E223K, E223Y, E223W, E223F, E223G, E223C, E223R, N235NGSGAGGSY, E260Q, E260S, D261N, D261S, D269N, D269S, D269A, T310A, T310G, P316G, P316D, Y325F, Y325H, Y325L, Y325M, Y325I, Y325W, H328F, H328Y, H328Q, H328E, H328N, H328D, H328S, H328T, H329F, H329Y, H329Q, H329E, H329N, H329D, H329S, H329T, K336R, K336H, K336M, K336L, R340K, R340H, R340Q, R340E, Y347F, Y347H, Y347M, Y347L, Y347I, F351H, F351Y, F351M, F351L, F351I, F351W, F352H, F352Y, F352M, F352L, F352I, F352W, I363F, I363L, I363M, I363V, I363A, I363T, D364E, D364Q, D364H, D364N, D364S, D364T, R403K, R403H, R403Q, R403E, P411A, P411G, Q413N, Q413A, I414V, I414A, Y430F, Y430V, F432H, F432V, I448L, I448M, I448V, I448A, Y450H, Y450L, Y450F, P454A, P454G, P454S, P454T, P454V, L455I, L455V, L455M, L455T, L455N, L455F, L455A, R457K, R457H, R457Q, R457E, R457S, N459D, N459E, N459Q, N459H, N459S, N459T, N459A, S460A, S460G, S460T, S460V, R461K, R461H, R461Q, R461E, R461S, F462Y, F462H, F462L, F462I, F462V, F462W, R467K, R467H, R467Q, R467E, T472A, T472P, T472S, T472V, F491H, F491L, M495K, M495Q, M495L, N497D, N497Q, N497E, N497S, Y515F, Y515H, Y515M, Y515L, R557K, R557S, G582A, G582S, G582T, G582V, R653H, R653N, T657S, T657A, T657N, R661K, R661H, R661Q, R661E, Q695E, Q695N, Q695D, H698F, H698Q, H698N, S719A, S719T, S719V, L720I, L720V, L720M, L720T, L720N, L720F, L720A, H721F, H721Y, H721Q, H721E, H721N, H721D, H721S, H721T, K735R, K735Q, K735H, K735M, K735L, L738I, L738V, L738M, L738T, L738N, L738F, L738A, K742R, K742Q, K742H, K742M, K742L, M751L, M751I, M751V, M751T, M751K, R765K, R765H, R765Q, S777A, S777N, S777D, E779Q, E779H, E779D, E779N, D850N, D850S, D850A, Q894E, Q894N, Q894S, E910Q, E910S, L911V, L911A, F916H, F916L, F916M, F916I, F916A, K918Q, K918R, K918H, K918M, K918L, Q920E, Q920N, Q920S, Q920A, T924S, T924A, R925K, R925H, R925Q, Q926K, Q926E, Q926N, Q926D, K929R, K929H, K929Q, Q933E, Q933H, Q933K, Q933N, R951Q, R951S, S960A, S964A, K968Q, K968H, K968M, K968L, R976K, R976Q, R976H, V982A, V982T, V982L, V982I, Y1013F, Y1013H, G1030A, G1030S, G1030T, G1030V, A1032G, A1032S, A1032T, A1032V, K1085R, K1085Q, K1085H, K1085M, K1085L, M1089L, M1089I, M1089V, M1089T, M1089K, P1090A, P1090G, P1090S, P1090T, P1090V, Q1091D, Q1091S, Q1091E, Q1091H, Q1091K, Q1091N, T1098A, T1098P, T1098S, T1098V, E1099, E1099Q, E1099D, E1099N, E1099H, V1100A, V1100G, V1100T, V1100S, V1100I, V1100L, T1102A, T1102P, T1102S, T1102V, G1103A, G1103P, G1103S, F1105Y, F1105H, F1105L, F1105I, F1105V, F1105W, K1107R, K1107Q, K1107M, E1108Q, E1108D, E1108N, S1109A, S1109T, S1109N, S1109D, K1113R, K1113Q, K1113H, K1113M, K1113L, R1114K, R1114Q, R1114H, N1115D, N1115A, N1115S, S1116A, D1117N, D1117S, D1117A, K1118Q, K1118H, K1118M, K1118L, K1123R, K1123Q, K1123H, K1123M, K1123L, K1123S, K1124R, K1124Q, K1124H, K1124M, K1124L, W1126Y, W1126S, F1134Y, F1134H, F1134L, F1134I, F1134V, F1134W, D1135N, D1135S, D1135A, R1171K, R1171H, R1171Q, R1171E, K1197R, K1197Q, K1197H, K1197M, K1197L, K1211R, K1211Q, K1211H, K1211M, K1211L, S1216A, E1219Q, E1219D, E1219N, E1243Q, E1243D, E1243N, E1243S, E1243A, R1279K, R1279H, R1279Q, R1279E, D1284N, D1284S, D1284A, P1301S, P1301G, H1311Y, H1311S, R1333K, R1333Q, R1333H, K1334R, K1334Q, K1334M, K1334L, R1335K, R1335Q, R1335H, T1337A, T1337S, S1338A, H1349F, H1349Y, H1349Q, H1349E, H1349N, H1349D, H1349S, H1349T, Y1356F, Y1356W, Y1356H, Y1356Q, Y1356E, Y1356N, Y1356D, Y1356S, Y1356T, T1358A, T1358P, T1358S, and T1358V.

12) The tsCas9 according to embodiment 11, wherein the at least one amino acid alteration is selected from the group consisting of P176A, P176G, P176S, P176T, P176V, S179A, S179G, S179T, S179V, S179P, E223Q, E223D, E223N, E223H, E223S, E223A, E223I, E223L, E223T, E223V, E223P, E223K, E223Y, E223W, E223F, E223G, E223C, E223R, E260Q, E260S, D261N, D261S, T310A, T310G, P316G, P316D, P411A, P411G, Q413N, Q413A, I414V, I414A, Y430F, Y430V, F432H, F432V, R457K, R457H, R457Q, R457E, R457S, N459D, N459E, N459Q, N459H, N459S, N459T, N459A, R461K, R461H, R461Q, R461E, R461S, R557K, R557S, R653H, R653N, E910Q, E910S, L911V, L911A, R951Q, R951S, K1123R, K1123Q, K1123H, K1123M, K1123L, K1123S, P1301S, P1301G, H1311Y, and H1311S.

13) The tsCas9 according to embodiment 11, wherein the at least one amino acid alteration is selected from the group consisting of S29A, S29G, S29T, S29V, K31R, K31Q, K31H, K31M, K31L, F32Y, F32H, F32H, F32L, F32I, F32M, L35V, L35A, L35T, L35I, L35M, L35F, L35H, K44R, K44Q, K44H, K44M, K44L, E57Q, K44D, K44N, K44H, K44M, K44L, N46D, N46S, N46T, N46Q, E57Q, E57D, E57N, E57H, T62S, T62A, T62V, T62P, R63K, R63H, R63Q, R63E, K65R, K65H, K65Q, K65M, K65L, R66K, R66H, R66Q, R66E, R69K, R69H, R69Q, R69E, R70K, R70H, R70Q, R70E, R71K, R71H, R71Q, R71E, Y72F, Y72H, Y72L, Y72M, Y72I, Y72W, R74K, R74H, R74Q, R74E, R75K, R75H, R75Q, R75E, R78K, R78H, R78Q, R78E, S104A, S104A, S104T, S104V, F105H, F105L, F105M, F105I, F105Y, F105W, V107S, V107A, V107I, V107L, V107T, E108Q, E108D, E108N, R115K, R115H, R115Q, R115E, H116F, H116Y, H116Q, H116E, H116N, H116D, H116S, H116T, V126I, V126L, V126A, V126T, V126S, H129F, H129Y, H129Q, H129E, H129N, H129D, H129S, H129T, Y136F, Y136H, Y136L, Y136M, Y136I, Y136W, H160F, H160Y, H160Q, H160E, H160N, H160D, H160S, H160T, K163R, K163H, K163Q, K163E, K163M, K163L, F164Y, F164H, F164M, F164L, F164I, F164W, R165K, R165H, R165Q, R165E, Y325F, Y325H, Y325L, Y325M, Y325I, Y325W, H328F, H328Y, H328Q, H328E, H328N, H328D, H328S, H328T, H329F, H329Y, H329Q, H329E, H329N, H329D, H329S, H329T, K336R, K336H, K336M, K336L, R340K, R340H, R340Q, R340E, Y347F, Y347H, Y347M, Y347L, Y347I, F351H, F351Y, F351M, F351L, F351I, F351W, F352H, F352Y, F352M, F352L, F352I, F352W, I363F, I363L, I363M, I363V, I363A, I363T, D364E, D364Q, D364H, D364N, D364S, D364T, R403K, R403H, R403Q, R403E, I448L, I448M, I448V, I448A, P454A, P454G, P454S, P454T, P454V, L455I, L455V, L455M, L455T, L455N, L455F, L455A, R457K, R457H, R457Q, R457E, R457S, N459D, N459E, N459Q, N459H, N459S, N459T, N459A, S460A, S460G, S460T, S460V, R461K, R461H, R461Q, R461E, R461S, F462Y, F462H, F462L, F462I, F462V, F462W, R467K, R467H, R467Q, R467E, T472A, T472P, T472S, T472V, Y515F, Y515H, Y515M, Y515L, R661K, R661H, R661Q, R661E, S719A, S719T, S719V, L720I, L720V, L720M, L720T, L720N, L720F, L720A, H721F, H721Y, H721Q, H721E, H721N, H721D, H721S, H721T, K735R, K735Q, K735H, K735M, K735L, L738I, L738V, L738M, L738T, L738N, L738F, L738A, K742R, K742Q, K742H, K742M, K742L, M751L, M751I, M751V, M751T, M751K, S777A, S777N, S777D, E779Q, E779H, E779D, E779N, D850N, D850S, D850A, K918Q, K918R, K918H, K918M, K918L, Q933E, Q933H, Q933K, Q933N, V982A, V982T, V982L, V982I, K1085R, K1085Q, K1085H, K1085M, K1085L, M1089L, M1089I, M1089V, M1089T, M1089K, P1090A, P1090G, P1090S, P1090T, P1090V, Q1091D, Q1091S, Q1091E, Q1091H, Q1091K, Q1091N, T1098A, T1098P, T1098S, T1098V, E1099, E1099Q, E1099D, E1099N, E1099H, V1100A, V1100G, V1100T, V1100S, V1100I, V1100L, T1102A, T1102P, T1102S, T1102V, G1103A, G1103P, G1103S, F1105Y, F1105H, F1105L, F1105I, F1105V, F1105W, E1108Q, E1108D, E1108N, K1113R, K1113Q, K1113H, K1113M, K1113L, K1123R, K1123Q, K1123H, K1123M, K1123L, K1123S, K1124R, K1124Q, K1124H, K1124M, K1124L, F1134Y, F1134H, F1134L, F1134I, F1134V, F1134W, R1171K, R1171H, R1171Q, R1171E, K1197R, K1197Q, K1197H, K1197M, K1197L, K1211R, K1211Q, K1211H, K1211M, K1211L, R1279K, R1279H, R1279Q, R1279E, H1349F, H1349Y, H1349Q, H1349E, H1349N, H1349D, H1349S, H1349T, Y1356F, Y1356W, Y1356H, Y1356Q, Y1356E, Y1356N, Y1356D, Y1356S, Y1356T, T1358A, T1358P, T1358S, and T1358V.

14) The tsCas9 according to embodiment 11, wherein the at least one amino acid alteration is selected from the group consisting of T13S, T13A, N14D, N14S, N14A, S55A, D269N, D269S, D269A, Y450H, Y450L, Y450F, F491H, F491L, M495K, M495Q, M495L, N497D, N497Q, N497E, N497S, G582A, G582S, G582T, G582V, T657S, T657A, T657N, R661K, R661H, R661Q, R661E, Q695E, Q695N, Q695D, H698F, H698Q, H698N, R765K, R765H, R765Q, S777A, S777N, S777D, Q894E, Q894N, Q894S, F916H, F916L, F916M, F916I, F916A, K918Q, K918R, K918H, K918M, K918L, Q920E, Q920N, Q920S, Q920A, T924S, T924A, R925K, R925H, R925Q, Q926K, Q926E, Q926N, Q926D, K929R, K929H, K929Q, S960A, S964A, K968Q, K968H, K968M, K968L, R976K, R976Q, R976H, Y1013F, Y1013H, G1030A, G1030S, G1030T, G1030V, A1032G, A1032S, A1032T, A1032V, K1107R, K1107Q, K1107M, E1108Q, E1108D, E1108N, S1109A, S1109T, S1109N, S1109D, R1114K, R1114Q, R1114H, N1115D, N1115A, N1115S, S1116A, D1117N, D1117S, D1117A, K1118Q, K1118H, K1118M, K1118L, D1135N, D1135S, D1135A, S1216A, E1219Q, E1219D, E1219N, E1243Q, E1243D, E1243N, E1243S, E1243A, D1284N, D1284S, D1284A, R1333K, R1333Q, R1333H, K1334R, K1334Q, K1334M, K1334L, R1335K, R1335Q, R1335H, T1337A, T1337S, and S1338A.

15) The tsCas9 according to embodiment 11, wherein the at least one amino acid alteration is selected from the group consisting of S29A, S29G, S29T, S29V, K31R, K31Q, K31H, K31M, K31L, F32Y, F32H, F32H, F32L, F32I, F32M, L35V, L35A, L35T, L35I, L35M, L35F, L35H, K44R, K44Q, K44H, K44M, K44L, E57Q, K44D, K44N, K44H, K44M, K44L, N46D, N46S, N46T, N46Q, E57Q, E57D, E57N, E57H, T62S, T62A, T62V, T62P, R63K, R63H, R63Q, R63E, K65R, K65H, K65Q, K65M, K65L, R66K, R66H, R66Q, R66E, R69K, R69H, R69Q, R69E, R70K, R70H, R70Q, R70E, R71K, R71H, R71Q, R71E, Y72F, Y72H, Y72L, Y72M, Y72I, Y72W, R74K, R74H, R74Q, R74E, R75K, R75H, R75Q, R75E, R78K, R78H, R78Q, R78E, S104A, S104A, S104T, S104V, F105H, F105L, F105M, F105I, F105Y, F105W, V107S, V107A, V107I, V107L, V107T, E108Q, E108D, E108N, R115K, R115H, R115Q, R115E, H116F, H116Y, H116Q, H116E, H116N, H116D, H116S, H116T, V126I, V126L, V126A, V126T, V126S, H129F, H129Y, H129Q, H129E, H129N, H129D, H129S, H129T, Y136F, Y136H, Y136L, Y136M, Y136I, Y136W, H160F, H160Y, H160Q, H160E, H160N, H160D, H160S, H160T, K163R, K163H, K163Q, K163E, K163M, K163L, F164Y, F164H, F164M, F164L, F164I, F164W, R165K, R165H, R165Q, R165E, P176A, P176G, P176S, P176T, P176V, S179A, S179G, S179T, S179V, S179P, E223Q, E223D, E223N, E223H, E223S, E223A, E223I, E223L, E223T, E223V, E223P, E223K, E223Y, E223W, E223F, E223G, E223C, E223R, E260Q, E260S, D261N, D261S, T310A, T310G, P316G, P316D, Y325F, Y325H, Y325L, Y325M, Y325I, Y325W, H328F, H328Y, H328Q, H328E, H328N, H328D, H328S, H328T, H329F, H329Y, H329Q, H329E, H329N, H329D, H329S, H329T, K336R, K336H, K336M, K336L, R340K, R340H, R340Q, R340E, Y347F, Y347H, Y347M, Y347L, Y347I, F351H, F351Y, F351M, F351L, F351I, F351W, F352H, F352Y, F352M, F352L, F352I, F352W, I363F, I363L, I363M, I363V, I363A, I363T, D364E, D364Q, D364H, D364N, D364S, D364T, R403K, R403H, R403Q, R403E, P411A, P411G, Q413N, Q413A, I414V, I414A, Y430F, Y430V, F432H, F432V, I448L, I448M, I448V, I448A, P454A, P454G, P454S, P454T, P454V, L455I, L455V, L455M, L455T, L455N, L455F, L455A, R457K, R457H, R457Q, R457E, R457S, N459D, N459E, N459Q, N459H, N459S, N459T, N459A, S460A, S460G, S460T, S460V, R461K, R461H, R461Q, R461E, R461S, F462Y, F462H, F462L, F462I, F462V, F462W, R467K, R467H, R467Q, R467E, T472A, T472P, T472S, T472V, Y515F, Y515H, Y515M, Y515L, R557K, R557S, R653H, R653N, R661K, R661H, R661Q, R661E, S719A, S719T, S719V, L720I, L720V, L720M, L720T, L720N, L720F, L720A, H721F, H721Y, H721Q, H721E, H721N, H721D, H721S, H721T, K735R, K735Q, K735H, K735M, K735L, L738I, L738V, L738M, L738T, L738N, L738F, L738A, K742R, K742Q, K742H, K742M, K742L, M751L, M751I, M751V, M751T, M751K, S777A, S777N, S777D, E779Q, E779H, E779D, E779N, D850N, D850S, D850A, E910Q, E910S, L911V, L911A, K918Q, K918R, K918H, K918M, K918L, Q933E, Q933H, Q933K, Q933N, R951Q, R951S, V982A, V982T, V982L, V982I, K1085R, K1085Q, K1085H, K1085M, K1085L, M1089L, M1089I, M1089V, M1089T, M1089K, P1090A, P1090G, P1090S, P1090T, P1090V, Q1091D, Q1091S, Q1091E, Q1091H, Q1091K, Q1091N, T1098A, T1098P, T1098S, T1098V, E1099, E1099Q, E1099D, E1099N, E1099H, V1100A, V1100G, V1100T, V1100S, V1100I, V1100L, T1102A, T1102P, T1102S, T1102V, G1103A, G1103P, G1103S, F1105Y, F1105H, F1105L, F1105I, F1105V, F1105W, E1108Q, E1108D, E1108N, K1113R, K1113Q, K1113H, K1113M, K1113L, K1123R, K1123Q, K1123H, K1123M, K1123L, K1123S, K1124R, K1124Q, K1124H, K1124M, K1124L, F1134Y, F1134H, F1134L, F1134I, F1134V, F1134W, R1171K, R1171H, R1171Q, R1171E, K1197R, K1197Q, K1197H, K1197M, K1197L, K1211R, K1211Q, K1211H, K1211M, K1211L, R1279K, R1279H, R1279Q, R1279E, P1301S, P1301G, H1311Y, H1311S, H1349F, H1349Y, H1349Q, H1349E, H1349N, H1349D, H1349S, H1349T, Y1356F, Y1356W, Y1356H, Y1356Q, Y1356E, Y1356N, Y1356D, Y1356S, Y1356T, T1358A, T1358P, T1358S, and T1358V.

16) The tsCas9 according to embodiment 11, wherein the at least one amino acid alteration is selected from the group consisting of T13S, T13A, N14D, N14S, N14A, S55A, P176A, P176G, P176S, P176T, P176V, S179A, S179G, S179T, S179V, S179P, E223Q, E223D, E223N, E223H, E223S, E223A, E223I, E223L, E223T, E223V, E223P, E223K, E223Y, E223W, E223F, E223G, E223C, E223R, E260Q, E260S, D261N, D261S, D269N, D269S, D269A, T310A, T310G, P316G, P316D, P411A, P411G, Q413N, Q413A, I414V, I414A, Y430F, Y430V, F432H, F432V, Y450H, Y450L, Y450F, R457K, R457H, R457Q, R457E, R457S, N459D, N459E, N459Q, N459H, N459S, N459T, N459A, R461K, R461H, R461Q, R461E, R461S, F491H, F491L, M495K, M495Q, M495L, N497D, N497Q, N497E, N497S, R557K, R557S, G582A, G582S, G582T, G582V, R653H, R653N, T657S, T657A, T657N, R661K, R661H, R661Q, R661E, Q695E, Q695N, Q695D, H698F, H698Q, H698N, R765K, R765H, R765Q, S777A, S777N, S777D, Q894E, Q894N, Q894S, E910Q, E910S, L911V, L911A, F916H, F916L, F916M, F916I, F916A, K918Q, K918R, K918H, K918M, K918L, Q920E, Q920N, Q920S, Q920A, T924S, T924A, R925K, R925H, R925Q, Q926K, Q926E, Q926N, Q926D, K929R, K929H, K929Q, R951Q, R951S, S960A, S964A, K968Q, K968H, K968M, K968L, R976K, R976Q, R976H, Y1013F, Y1013H, G1030A, G1030S, G1030T, G1030V, A1032G, A1032S, A1032T, A1032V, K1107R, K1107Q, K1107M, E1108Q, E1108D, E1108N, S1109A, S1109T, S1109N, S1109D, R1114K, R1114Q, R1114H, N1115D, N1115A, N1115S, S1116A, D1117N, D1117S, D1117A, K1118Q, K1118H, K1118M, K1118L, K1123R, K1123Q, K1123H, K1123M, K1123L, K1123S, D1135N, D1135S, D1135A, S1216A, E1219Q, E1219D, E1219N, E1243Q, E1243D, E1243N, E1243S, E1243A, D1284N, D1284S, D1284A, P1301S, P1301G, H1311Y, H1311S, R1333K, R1333Q, R1333H, K1334R, K1334Q, K1334M, K1334L, R1335K, R1335Q, R1335H, T1337A, T1337S, and S1338A.

17) The tsCas9 according to embodiment 11, wherein the at least one amino acid alteration is selected from the group consisting of T13S, T13A, N14D, N14S, N14A, S29A, S29G, S29T, S29V, K31R, K31Q, K31H, K31M, K31L, F32Y, F32H, F32H, F32L, F32I, F32M, L35V, L35A, L35T, L35I, L35M, L35F, L35H, K44R, K44Q, K44H, K44M, K44L, E57Q, K44D, K44N, K44H, K44M, K44L, N46D, N46S, N46T, N46Q, S55A, E57Q, E57D, E57N, E57H, T62S, T62A, T62V, T62P, R63K, R63H, R63Q, R63E, K65R, K65H, K65Q, K65M, K65L, R66K, R66H, R66Q, R66E, R69K, R69H, R69Q, R69E, R70K, R70H, R70Q, R70E, R71K, R71H, R71Q, R71E, Y72F, Y72H, Y72L, Y72M, Y72I, Y72W, R74K, R74H, R74Q, R74E, R75K, R75H, R75Q, R75E, R78K, R78H, R78Q, R78E, S104A, S104A, S104T, S104V, F105H, F105L, F105M, F105I, F105Y, F105W, V107S, V107A, V107I, V107L, V107T, E108Q, E108D, E108N, R115K, R115H, R115Q, R115E, H116F, H116Y, H116Q, H116E, H116N, H116D, H116S, H116T, V126I, V126L, V126A, V126T, V126S, H129F, H129Y, H129Q, H129E, H129N, H129D, H129S, H129T, Y136F, Y136H, Y136L, Y136M, Y136I, Y136W, H160F, H160Y, H160Q, H160E, H160N, H160D, H160S, H160T, K163R, K163H, K163Q, K163E, K163M, K163L, F164Y, F164H, F164M, F164L, F164I, F164W, R165K, R165H, R165Q, R165E, D269N, D269S, D269A, Y325F, Y325H, Y325L, Y325M, Y325I, Y325W, H328F, H328Y, H328Q, H328E, H328N, H328D, H328S, H328T, H329F, H329Y, H329Q, H329E, H329N, H329D, H329S, H329T, K336R, K336H, K336M, K336L, R340K, R340H, R340Q, R340E, Y347F, Y347H, Y347M, Y347L, Y347I, F351H, F351Y, F351M, F351L, F351I, F351W, F352H, F352Y, F352M, F352L, F352I, F352W, I363F, I363L, I363M, I363V, I363A, I363T, D364E, D364Q, D364H, D364N, D364S, D364T, R403K, R403H, R403Q, R403E, I448L, I448M, I448V, I448A, Y450H, Y450L, Y450F, P454A, P454G, P454S, P454T, P454V, L455I, L455V, L455M, L455T, L455N, L455F, L455A, R457K, R457H, R457Q, R457E, R457S, N459D, N459E, N459Q, N459H, N459S, N459T, N459A, S460A, S460G, S460T, S460V, R461K, R461H, R461Q, R461E, R461S, F462Y, F462H, F462L, F462I, F462V, F462W, R467K, R467H, R467Q, R467E, T472A, T472P, T472S, T472V, F491H, F491L, M495K, M495Q, M495L, N497D, N497Q, N497E, N497S, Y515F, Y515H, Y515M, Y515L, G582A, G582S, G582T, G582V, T657S, T657A, T657N, R661K, R661H, R661Q, R661E, Q695E, Q695N, Q695D, H698F, H698Q, H698N, S719A, S719T, S719V, L720I, L720V, L720M, L720T, L720N, L720F, L720A, H721F, H721Y, H721Q, H721E, H721N, H721D, H721S, H721T, K735R, K735Q, K735H, K735M, K735L, L738I, L738V, L738M, L738T, L738N, L738F, L738A, K742R, K742Q, K742H, K742M, K742L, M751L, M751I, M751V, M751T, M751K, R765K, R765H, R765Q, S777A, S777N, S777D, E779Q, E779H, E779D, E779N, D850N, D850S, D850A, Q894E, Q894N, Q894S, F916H, F916L, F916M, F916I, F916A, K918Q, K918R, K918H, K918M, K918L, Q920E, Q920N, Q920S, Q920A, T924S, T924A, R925K, R925H, R925Q, Q926K, Q926E, Q926N, Q926D, K929R, K929H, K929Q, Q933E, Q933H, Q933K, Q933N, S960A, S964A, K968Q, K968H, K968M, K968L, R976K, R976Q, R976H, V982A, V982T, V982L, V982I, Y1013F, Y1013H, G1030A, G1030S, G1030T, G1030V, A1032G, A1032S, A1032T, A1032V, K1085R, K1085Q, K1085H, K1085M, K1085L, M1089L, M1089I, M1089V, M1089T, M1089K, P1090A, P1090G, P1090S, P1090T, P1090V, Q1091D, Q1091S, Q1091E, Q1091H, Q1091K, Q1091N, T1098A, T1098P, T1098S, T1098V, E1099, E1099Q, E1099D, E1099N, E1099H, V1100A, V1100G, V1100T, V1100S, V1100I, V1100L, T1102A, T1102P, T1102S, T1102V, G1103A, G1103P, G1103S, F1105Y, F1105H, F1105L, F1105I, F1105V, F1105W, K1107R, K1107Q, K1107M, E1108Q, E1108D, E1108N, S1109A, S1109T, S1109N, S1109D, K1113R, K1113Q, K1113H, K1113M, K1113L, R1114K, R1114Q, R1114H, N1115D, N1115A, N1115S, S1116A, D1117N, D1117S, D1117A, K1118Q, K1118H, K1118M, K1118L, K1123R, K1123Q, K1123H, K1123M, K1123L, K1123S, K1124R, K1124Q, K1124H, K1124M, K1124L, F1134Y, F1134H, F1134L, F1134I, F1134V, F1134W, D1135N, D1135S, D1135A, R1171K, R1171H, R1171Q, R1171E, K1197R, K1197Q, K1197H, K1197M, K1197L, K1211R, K1211Q, K1211H, K1211M, K1211L, S1216A, E1219Q, E1219D, E1219N, E1243Q, E1243D, E1243N, E1243S, E1243A, R1279K, R1279H, R1279Q, R1279E, D1284N, D1284S, D1284A, R1333K, R1333Q, R1333H, K1334R, K1334Q, K1334M, K1334L, R1335K, R1335Q, R1335H, T1337A, T1337S, S1338A, H1349F, H1349Y, H1349Q, H1349E, H1349N, H1349D, H1349S, H1349T, Y1356F, Y1356W, Y1356H, Y1356Q, Y1356E, Y1356N, Y1356D, Y1356S, Y1356T, T1358A, T1358P, T1358S, and T1358V.

18) The tsCas9 according to embodiment 11, wherein the at least one amino acid alteration is selected from the group consisting of S104A, F105H, V107S, P176S, S179A, E223A, N235NGSGAGGSY, E260Q, D261N, T310A, T310G, P316G, P316D, P411A, P411G, Q413N, Q413A, I414V, I414A, Y430F, Y430V, F432H, F432V, R457K, N459S, P475S, W476H, R557K, R653H, Q739S, E910S, L911A, R951Q, K1123S, W1126S, and H1311Y; preferably the at least one amino acid alteration is selected from the group consisting of S104A, F105H, V107S, P176S, S179A, E223A, E260Q, D261N, T310A, T310G, P316G, P316D, P411A, P411G, Q413N, Q413A, I414V, I414A, Y430F, Y430V, F432H, F432V, R457K, N459S, P475S, W476H, R557K, R653H, Q739S, E910S, L911A, R951Q, K1123S, W1126S, and H1311Y.

19) The tsCas9 according to embodiment 11, wherein the at least one amino acid alteration include N235NGSGAGGSY; S104A, F105H, and V107S; P176S and S179A; E223A; T310A and P316G; P411A, Q413N, and I414V; P411A, Q413N, I414V, R457K, and N495S; Y430F and F432H; R457K and N495S; P475S and W476H; R557K; R653H; R951Q; S104A, F105H, V107S, P176S, S179A, E223S, E260Q, D261N, R653H, Q739S, and H1311Y; S104A, F105H, V107S, E260Q, D261N, T310A, P316G, Y430F, F432H, Q739S, and H1311Y; S104A, F105H, V107S, T310A, P316G, Q739S, and R951Q; S104A, F105H, V107S, E260Q, D261N, Y430F, F432H, and Q739S; S104A, F105H, V107S, Q739S, R951Q, and H1311Y; S104A, F105H, V107S, P411A, Q413N, and I414V; S104A, F105H, V107S, P411A, Q413N, I414V, R457K, and N459S; T310G and P316D; P411G, Q413A, and I414A; Y430V and F432V; E910S and L911A; and K1123S and W1126S; preferably the at least one amino acid alteration include S104A, F105H, and V107S; P176S and S179A; E223A; T310A and P316G; P411A, Q413N, and I414V; P411A, Q413N, I414V, R457K, and N495S; Y430F and F432H; R457K and N495S; P475S and W476H; R557K; R653H; R951Q; S104A, F105H, V107S, P176S, S179A, E223S, E260Q, D261N, R653H, Q739S, and H1311Y; S104A, F105H, V107S, E260Q, D261N, T310A, P316G, Y430F, F432H, Q739S, and H1311Y; S104A, F105H, V107S, T310A, P316G, Q739S, and R951Q; S104A, F105H, V107S, E260Q, D261N, Y430F, F432H, and Q739S; S104A, F105H, V107S, Q739S, R951Q, and H1311Y; S104A, F105H, V107S, P411A, Q413N, and I414V; S104A, F105H, V107S, P411A, Q413N, I414V, R457K, and N459S; T310G and P316D; P411G, Q413A, and I414A; Y430V and F432V; E910S and L911A; and K1123S and W1126S.

20) The tsCas9 according to any of the preceding embodiments, which has a permissive temperature range selected from the group consisting of 30-32° C., 30-33° C., 29-32° C., 30-34° C., 29-33° C., 30-35° C., 29-34° C., 30-36° C., 28-34° C., 31-35° C., 28-35° C., 26-34° C., and 27-34° C.; preferably, the permissive temperature range is selected from the group consisting of 30-32° C., 29.5-32.5° C., 29-33° C., 28.5-33.5° C., 28-34° C., 27.5-34.5° C., 27-35° C., 26.5-35.5° C., and 26-36° C.; more preferably, the permissive temperature range is selected from the group consisting of 29-33° C., 28.5-33.5° C., 28-34° C., 27.5-34.5° C., and 27-35° C.; even more preferably, the permissive temperature range is selected from the group consisting of 28.5-33.5° C., 28-34° C., and 27.5-34.5° C.; most preferably, the permissive temperature range is 28-34° C.

21) The tsCas9 according to any of the preceding embodiments, which has a restrictive temperature range selected from the group consisting of 38-40° C., 38-41° C., 37-40° C., 38-42° C., 37-41° C., 37-42° C., 36-40° C., 36-41° C., 36-42° C., and 35-43° C.; preferably, the restrictive temperature range is selected from the group consisting of 39-40° C., 38.5-41.5° C., 38-42° C., 37.5-42° C., 38-42.5° C., 36.5-41.5° C., 36.5-42° C., 37-42° C., 37-42.5° C., and 36-43° C.; more preferably, the restrictive temperature range is selected from the group consisting of 38-42° C., 37.5-42° C., 38-42.5° C., 36.5-41.5° C., 36.5-42° C., 37-42° C., 37-42.5° C., 36-43° C., and 36-44° C.; even more preferably, the restrictive temperature range is selected from the group consisting of 36.5-41.5° C., 36.5-42° C., 37-42° C., and 37-42.5° C.; most preferably, the restrictive temperature range is 37-42° C.

22) The tsCas9 according to any of the preceding embodiments, which has a dissociative temperature range selected from the group consisting of 40-50° C., 41-50° C., 42-50° C., 43-50° C., 44-50° C., and 45-50° C.; preferably, the dissociative temperature range is selected from the group consisting of 41-50° C., 42-50° C., and 43-50° C.; more preferably, the dissociative temperature range is selected from the group consisting of 41-50° C., 41.5-50° C., 42-50° C., 42.5-50° C., and 43-50° C.; even more preferably, the dissociative temperature range is selected from the group consisting of 41.5-50° C., 42-50° C., and 42.5-50° C.; most preferably, the dissociative temperature range is 42-50° C.

23) A polynucleotide encoding a tsCas9 according to any of embodiments 1-22.

24) The polynucleotide according to embodiment 23, said polynucleotide having at least 80% sequence identity to SEQ ID NO:1.

25) A nucleic acid construct comprising a polynucleotide according to any of embodiments 23-24.

26) An expression vector comprising a polynucleotide according to any of embodiments 23-24.

27) A host cell comprising a tsCas9 as defined in any of embodiments 1-22, a polynucleotide as defined in any of embodiments 23-24, a nucleic acid construct as defined in embodiment 25, and/or an expression vector as defined in embodiment 26.

28) The host cell according to embodiment 27, said host cell being a eukaryotic or prokaryotic host cell.

29) The host cell according to any of embodiments 27-28, said host cell being a microorganism host cell selected from the group consisting of bacterial, fungal, yeast, and archeal host cell.

30) The host cell according to any of embodiments 27-29, said host cell being a bacterial host cell selected from the group consisting of Bacillus, Escherichia, Lactobacillus, Lactococcus Streptococcus, and Streptomyces cell; preferably the host cell is selected from the group consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, Bacillus thuringiensis, Escherichia coli, Lactobacillus acidophilus, Lactobacillus amylovorus, Lactobacillus brevis, Lactobacillus (para)casei, Lactobacillus cellobiosus, Lactobacillus crispatus, Lactobacillus curvatus, Lactobacillus delbrueckii subsp. bulgaricus, L. delbrueckii subsp. lactis, Lactobacillus fermentum, Lactobacillus gallinarum, Lactobacillus gasseri, Lactobacillus helveticus, Lactobacillus johnsonii, Lactobacillus plantarum, Lactobacillus reuteri, Lactobacillus rhamnosus, Lactobacillus salivarius, Lactococcus chungangensis, Lactococcus formosensis, Lactococcus fujiensis, Lactococcus garvieae, Lactococcus lactis, Lactococcus piscium, Lactococcus plantarum, Lactococcus raffinolactis, Lactococcus taiwanensi, Streptococcus equisimilis, Streptococcus pyogenes, Streptococcus uberis, Streptococcus equi subsp. zooepidemicus, Streptomyces achromogenes, Streptomyces avermitilis, Streptomyces coelicolor, Streptomyces griseus, and Streptomyces lividans; more preferably the host cell is Bacillus licheniformis.

31) The host cell according to any of embodiments 27-30, said host cell being a fungal host cell selected from the group of consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell.

32) The host cell according to any of embodiments 27-31, said host cell being a yeast host cell selected from the group consisting of Candida, Hansenula, Kluyveromyces, Pichia, Saccharomyces, Schizosaccharomyces, and Yarrowia cell; preferably the host cell is selected from the group consisting of Kluyveromyces lactis, Pichia pastoris, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell.

33) The host cell of any of embodiments 27-32, wherein the tsCas9 is a nuclease-null variant; preferably said variant comprises an amino acid alteration in a position corresponding to position 10 and position 840 of SEQ ID NO:2; more preferably said variant comprises a substitution of aspartic acid for alanine, D10A, and a substitution of histidine for alanine, H840A.

34) A method of inducing expression of one or more genome target sequence of interest, the method comprising the steps of:
a) providing a host cell according to embodiment 33, said host cell further comprising one or more suitable gRNA and one or more genome target sequence of interest;
b) cultivating the host cell at a dissociative temperature of the tsCas9, whereby a complex formed in the host cell by the tsCas9 with the one or more suitable gRNA and the one or more genome target sequence of interest disassociates; and subsequently
c) lowering the temperature to a restrictive temperature of the tsCas9 and cultivating the host cell, whereby expression of the one or more target sequence is induced.

35) The method according to embodiment 34, wherein the dissociative temperature is 42-50° C. and the restrictive temperature is 37-42° C.

36) The method according to any of embodiments 34-35, said method comprising the following step after providing the host cell and before step (b) of: cultivating the host cell at a permissive temperature of the tsCas9, whereby a complex is formed in the host cell by the tsCas9, the one or more suitable gRNA and the one or more genome target sequence of interest, whereby expression of the one or more genome target sequence is repressed.

37) The method according to any of embodiments 34-36, said method comprising the additional step of:
d) increasing the temperature to a permissive temperature of the tsCas9, wherein a complex is formed in the host cell by the tsCas9, the one or more suitable gRNA, and the one or more genome target sequence of interest, and cultivating the host cell, whereby expression of the one or more genome target sequence of interest is repressed.

38) The method according to any of embodiments 36-37, wherein the permissive temperature is 28-34° C.

39) A method of repressing one or more genome target sequence of interest, the method comprising the steps of:
a) providing a host cell according to embodiment 33, said host cell further comprising one or more suitable gRNA and one or more genome target sequence of interest;
b) cultivating the host cell at a restrictive temperature of the tsCas9, wherein the one or more genome target sequence of interest is expressed; and subsequently
c) lowering the temperature to a permissive temperature of the tsCas9, whereby a complex is formed in the host cell by the tsCas9, the one or more suitable gRNA and the one or more genome target sequence of interest, and whereby expression of the one or more genome target sequence is repressed.

40) The method according to embodiment 39, wherein the permissive temperature is 28-34° C. and the restrictive temperature is 37-42° C.

41) The method according to any of embodiments 39-40, said method comprising the following step after providing the host cell and before step (b) of: cultivating the host cell at a dissociative temperature of the tsCas9 to ensure that no complex is formed in the host cell between the tsCas9, the one or more gRNA and the one or more genome target sequence of interest.

42) The method according to any of embodiments 39-41, said method comprising the additional steps of:
d) raising the temperature to a dissociative temperature of the tsCas9, whereby the complex disassociates; and subsequently
e) lowering the temperature to a restrictive temperature of the tsCas9, whereby expression of the one or more target sequence is induced.

43) The method according to any of embodiments 41-42, wherein the dissociative temperature is 42-50° C.

44) The method according to any of embodiments 34-43, wherein the one or more genome target sequence of interest comprises at least 20 nucleotides and further comprises or is flanked by a functional PAM sequence for a Class-II Cas9 protein; preferably the one or more genome target sequence is comprised in an open reading frame encoding a polypeptide or in a promoter region.

45) The method according to any of embodiments 34-44, wherein the one or more genome target sequence of interest encode one or more enzyme selected from the group consisting of hydrolase, isomerase, ligase, lyase, oxidoreductase, or a transferase; preferably the one or more enzyme is an alpha-amylase, alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, mannosidase, mutanase, oxidase, pectinolytic enzyme, peroxidase, phosphodiesterase, phytase, polyphenoloxidase, proteolytic enzyme, ribonuclease, transglutaminase, and xylanase.

46) The method according to any of embodiments 34-45, wherein the host cell is a eukaryotic or prokaryotic cell; preferably, the host cell is a microorganism host cell; even more preferably, the host cell is a fungal, bacterial, or archeal cell.

47) The method according to any of embodiments 34-46, wherein the host cell is a Bacillus host cell; preferably the host cell is selected from the group of Bacillus species consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis; more preferably the host cell is Bacillus licheniformis.

48) The method according to any of embodiments 34-47, wherein the one or more gRNA comprises a first RNA comprising 20 or more nucleotides that are at least 85% complementary to and capable of hybridizing to the one or more genome target sequence; preferably the 20 or more nucleotides are at least 90%, 95%, 97%, 98%, 99% or even 100% complementary to and capable of hybridizing to the one or more genome target sequence.

49) Use of a tsCas9 according to any of embodiments 1-22, a polynucleotide according to any of embodiments 23-24, a nucleic acid construct according to embodiment 25, an expression vector according to embodiment 26, a host cell according to any of embodiments 27-33, and/or a method according to any of embodiments 34-48 in medicine or cosmetics.

50) Use of a tsCas9 according to any of embodiments 1-22, a polynucleotide according to any of embodiments 23-24, a nucleic acid construct according to embodiment 25, an expression vector according to embodiment 26, a host cell according to any of embodiments 27-33, and/or a method according to any of embodiments 34-48 in medical or biotechnological research or production.

51) Use of a tsCas9 according to any of embodiments 1-22, a polynucleotide according to any of embodiments 23-24, a nucleic acid construct according to embodiment 25, an expression vector according to embodiment 26, a host cell according to any of embodiments 27-33, and/or a method according to any of embodiments 34-48 in genome editing, regulation of gene expression, or CRISPR inhibition.

52) Use of a tsCas9 according to any of embodiments 1-22, a polynucleotide according to any of embodiments 23-24, a nucleic acid construct according to embodiment 25, an expression vector according to embodiment 26, a host cell according to any of embodiments 27-33, and/or a method according to any of embodiments 34-48 in enzyme research, development, and/or production.

1. A temperature-sensitive variant of a Class-II Cas9 protein (tsCas9), said variant comprising at least one alteration of one or more amino acid important for protein stability or for stability of a complex formed between the Class-II Cas9 protein, one or more guide-RNA (gRNA), and one or more corresponding genome target sequence, wherein the at least one alteration is a substitution, insertion, or deletion of 1-10 amino acids; preferably the at least one alteration is a substitution or deletion of 1-10 amino acids; most preferably the at least one alteration is a substitution.

2. The tsCas9 according to claim 1, said variant being a nickase or nuclease-null variant; preferably, said tsCas9 comprising an alteration of an amino acid corresponding to position 10 and/or position 840 of SEQ ID NO:2; more preferably said variant comprises a substitution of aspartic acid for alanine, D10A, and/or a substitution of histidine for alanine, H840A.

3. The tsCas9 according to any of the preceding claims, wherein the at least one alteration is in a position corresponding to a position selected from the group consisting of T13, N14, S29, K31, F32, L35, K44, N46, S55, E57, T62, R63, K65, R66, R69, R70, R71, Y72, R74, R75, R78, S104, F105, V107, E108, R115, H116, V126, H129, Y136, H160, K163, F164, R165, P176, S179, E223, N235, E260, D261, D269, T310, P316, Y325, H328, H329, K336, R340, Y347, F351, F352, I363, D364, R403, P411, Q413, I414, Y430, F432, I448, Y450, P454, L455, R457, N459, S460, R461, F462, R467, T472, F491, M495 N497, Y515, R557, G582, R653, T657, R661, Q695, H698, S719, L720, H721, K735, L738, K742, M751, R765 S777, E779, D850, Q894, E910, L911, F916, K918, Q920, T924, R925, Q926, K929, Q933, R951, S960, S964, K968, R976, V982, Y1013, G1030, A1032, K1085, M1089, P1090, Q1091, T1098, E1099, V1100, T1102, G1103, F1105, K1107 E1108, S1109, K1113, R11414, N1115, S1116, D1117, K1118, K1123, K1124, F1134, D1135, W1126, K1135, R1171, K1197, K1211, S1216, E1219, E1243, R1279, D1284, P1301, H1311, R1333, K1334, R1335, T1337, S1338, H1349, Y1356, and T1358 of SEQ ID NO:2.

4. The tsCas9 according to any of the preceding claims, wherein the at least one alteration is in a position corresponding to a position selected from the group consisting of S104, F105, V107, P176, S179, E223, N235, E260, D261, T310, P316, P411, Q413, I414, Y430, F432, R457, N459, P475, W476, R557, R653, Q739, E910, L911, R951, K1123, W1126S, and H1311 of SEQ ID NO:2; preferably the at least one alteration is in a position corresponding to a position selected from the group consisting of S104, F105, V107, P176, S179, E223, E260, D261, T310, P316, P411, Q413, I414, Y430, F432, R457, N459, P475, W476, R557, R653, Q739, E910, L911, R951, K1123, W1126S, and H1311 of SEQ ID NO:2.

5. The tsCas9 according to any of claims 1-3, wherein the at least one amino acid alteration is selected from the group consisting of T13S, T13A, N14D, N14S, N14A, S29A, S29G, S29T, S29V, K31R, K31Q, K31H, K31M, K31L, F32Y, F32H, F32H, F32L, F32I, F32M, L35V, L35A, L35T, L35I, L35M, L35F, L35H, K44R, K44Q, K44H, K44M, K44L, K44D, K44N, K44H, K44M, K44L, N46D, N46S, N46T, N46Q, S55A, E57Q, E57D, E57N, E57H, T62S, T62A, T62V, T62P, R63K, R63H, R63Q, R63E, K65R, K65H, K65Q, K65M, K65L, R66K, R66H, R66Q, R66E, R69K, R69H, R69Q, R69E, R70K, R70H, R70Q, R70E, R71K, R71H, R71Q, R71E, Y72F, Y72H, Y72L, Y72M, Y72I, Y72W, R74K, R74H, R74Q, R74E, R75K, R75H, R75Q, R75E, R78K, R78H, R78Q, R78E, S104A, S104A, S104T, S104V, F105H, F105L, F105M, F105I, F105Y, F105W, V107S, V107A, V107I, V107L, V107T, E108Q, E108D, E108N, R115K, R115H, R115Q, R115E, H116F, H116Y, H116Q, H116E, H116N, H116D, H116S, H116T, V126I, V126L, V126A, V126T, V126S, H129F, H129Y, H129Q, H129E, H129N, H129D, H129S, H129T, Y136F, Y136H, Y136L, Y136M, Y136I, Y136W, H160F, H160Y, H160Q, H160E, H160N, H160D, H160S, H160T, K163R, K163H, K163Q, K163E, K163M, K163L, F164Y, F164H, F164M, F164L, F164I, F164W, R165K, R165H, R165Q, R165E, P176A, P176G, P176S, P176T, P176V, S179A, S179G, S179T, S179V, S179P, E223Q, E223D, E223N, E223H, E223S, E223A, E223I, E223L, E223T, E223V, E223P, E223K, E223Y, E223W, E223F, E223G, E223C, E223R, N235NGSGAGGSY, E260Q, E260S, D261N, D261S, D269N, D269S, D269A, T310A, T310G, P316G, P316D, Y325F, Y325H, Y325L, Y325M, Y325I, Y325W, H328F, H328Y, H328Q, H328E, H328N, H328D, H328S, H328T, H329F, H329Y, H329Q, H329E, H329N, H329D, H329S, H329T, K336R, K336H, K336M, K336L, R340K, R340H, R340Q, R340E, Y347F, Y347H, Y347M, Y347L, Y347I, F351H, F351Y, F351M, F351L, F351I, F351W, F352H, F352Y, F352M, F352L, F352I, F352W, I363F, I363L, I363M, I363V, I363A, I363T, D364E, D364Q, D364H, D364N, D364S, D364T, R403K, R403H, R403Q, R403E, P411A, P411G, Q413N, Q413A, I414V, I414A, Y430F, Y430V, F432H, F432V, I448L, I448M, I448V, I448A, Y450H, Y450L, Y450F, P454A, P454G, P454S, P454T, P454V, L455I, L455V, L455M, L455T, L455N, L455F, L455A, R457K, R457H, R457Q, R457E, R457S, N459D, N459E, N459Q, N459H, N459S, N459T, N459A, S460A, S460G, S460T, S460V, R461K, R461H, R461Q, R461E, R461S, F462Y, F462H, F462L, F462I, F462V, F462W, R467K, R467H, R467Q, R467E, T472A, T472P, T472S, T472V, F491H, F491L, M495K, M495Q, M495L, N497D, N497Q, N497E, N497S, Y515F, Y515H, Y515M, Y515L, R557K, R557S, G582A, G582S, G582T, G582V, R653H, R653N, T657S, T657A, T657N, R661K, R661H, R661Q, R661E, Q695E, Q695N, Q695D, H698F, H698Q, H698N, S719A, S719T, S719V, L720I, L720V, L720M, L720T, L720N, L720F, L720A, H721F, H721Y, H721Q, H721E, H721N, H721D, H721S, H721T, K735R, K735Q, K735H, K735M, K735L, L738I, L738V, L738M, L738T, L738N, L738F, L738A, K742R, K742Q, K742H, K742M, K742L, M751L, M751I, M751V, M751T, M751K, R765K, R765H, R765Q, S777A, S777N, S777D, E779Q, E779H, E779D, E779N, D850N, D850S, D850A, Q894E, Q894N, Q894S, E910Q, E910S, L911V, L911A, F916H, F916L, F916M, F916I, F916A, K918Q, K918R, K918H, K918M, K918L, Q920E, Q920N, Q920S, Q920A, T924S, T924A, R925K, R925H, R925Q, Q926K, Q926E, Q926N, Q926D, K929R, K929H, K929Q, Q933E, Q933H, Q933K, Q933N, R951Q, R951S, S960A, S964A, K968Q, K968H, K968M, K968L, R976K, R976Q, R976H, V982A, V982T, V982L, V982I, Y1013F, Y1013H, G1030A, G1030S, G1030T, G1030V, A1032G, A1032S, A1032T, A1032V, K1085R, K1085Q, K1085H, K1085M, K1085L, M1089L, M1089I, M1089V, M1089T, M1089K, P1090A, P1090G, P1090S, P1090T, P1090V, Q1091D, Q1091S, Q1091E, Q1091H, Q1091K, Q1091N, T1098A, T1098P, T1098S, T1098V, E1099, E1099Q, E1099D, E1099N, E1099H, V1100A, V1100G, V1100T, V1100S, V1100I, V1100L, T1102A, T1102P, T1102S, T1102V, G1103A, G1103P, G1103S, F1105Y, F1105H, F1105L, F1105I, F1105V, F1105W, K1107R, K1107Q, K1107M, E1108Q, E1108D, E1108N, S1109A, S1109T, S1109N, S1109D, K1113R, K1113Q, K1113H, K1113M, K1113L, R1114K, R1114Q, R1114H, N1115D, N1115A, N1115S, S1116A, D1117N, D1117S, D1117A, K1118Q, K1118H, K1118M, K1118L, K1123R, K1123Q, K1123H, K1123M, K1123L, K1123S, K1124R, K1124Q, K1124H, K1124M, K1124L, W1126Y, W1126S, F1134Y, F1134H, F1134L, F1134I, F1134V, F1134W, D1135N, D1135S, D1135A, R1171K, R1171H, R1171Q, R1171E, K1197R, K1197Q, K1197H, K1197M, K1197L, K1211R, K1211Q, K1211H, K1211M, K1211L, S1216A, E1219Q, E1219D, E1219N, E1243Q, E1243D, E1243N, E1243S, E1243A, R1279K, R1279H, R1279Q, R1279E, D1284N, D1284S, D1284A, P1301S, P1301G, H1311Y, H1311S, R1333K, R1333Q, R1333H, K1334R, K1334Q, K1334M, K1334L, R1335K, R1335Q, R1335H, T1337A, T1337S, S1338A, H1349F, H1349Y, H1349Q, H1349E, H1349N, H1349D, H1349S, H1349T, Y1356F, Y1356W, Y1356H, Y1356Q, Y1356E, Y1356N, Y1356D, Y1356S, Y1356T, T1358A, T1358P, T1358S, and T1358V.

6. The tsCas9 according to claim 5, wherein the at least one amino acid alteration is selected from the group consisting of S104A, F105H, V107S, P176S, S179A, E223A, N235NGSGAGGSY, E260Q, D261N, T310A, T310G, P316G, P316D, P411A, P411G, Q413N, Q413A, I414V, I414A, Y430F, Y430V, F432H, F432V, R457K, N459S, P475S, W476H, R557K, R653H, Q739S, E910S, L911A, R951Q, K1123S, W1126S, and H1311Y; preferably the at least one amino acid alteration is selected from the group consisting of S104A, F105H, V107S, P176S, S179A, E223A, E260Q, D261N, T310A, T310G, P316G, P316D, P411A, P411G, Q413N, Q413A, I414V, I414A, Y430F, Y430V, F432H, F432V, R457K, N459S, P475S, W476H, R557K, R653H, Q739S, E910S, L911A, R951Q, K1123S, W1126S, and H1311Y.

7. The tsCas9 according to any of claims 5-6, wherein the at least one amino acid alteration include N235NGSGAGGSY; S104A, F105H, and V107S; P176S and S179A; E223A; T310A and P316G; P411A, Q413N, and I414V; P411A, Q413N, I414V, R457K, and N495S; Y430F and F432H; R457K and N495S; P475S and W476H; R557K; R653H; R951Q; S104A, F105H, V107S, P176S, S179A, E223S, E260Q, D261N, R653H, Q739S, and H1311Y; S104A, F105H, V107S, E260Q, D261N, T310A, P316G, Y430F, F432H, Q739S, and H1311Y; S104A, F105H, V107S, T310A, P316G, Q739S, and R951Q; S104A, F105H, V107S, E260Q, D261N, Y430F, F432H, and Q739S; S104A, F105H, V107S, Q739S, R951Q, and H1311Y; S104A, F105H, V107S, P411A, Q413N, and I414V; S104A, F105H, V107S, P411A, Q413N, I414V, R457K, and N459S; T310G and P316D; P411G, Q413A, and I414A; Y430V and F432V; E910S and L911A; and K1123S W1126S; preferably the at least one amino acid alteration include S104A, F105H, and V107S; P176S and S179A; E223A; T310A and P316G; P411A, Q413N, and I414V; P411A, Q413N, I414V, R457K, and N495S; Y430F and F432H; R457K and N495S; P475S and W476H; R557K; R653H; R951Q; S104A, F105H, V107S, P176S, S179A, E223S, E260Q, D261N, R653H, Q739S, and H1311Y; S104A, F105H, V107S, E260Q, D261N, T310A, P316G, Y430F, F432H, Q739S, and H1311Y; S104A, F105H, V107S, T310A, P316G, Q739S, and R951Q; S104A, F105H, V107S, E260Q, D261N, Y430F, F432H, and Q739S; S104A, F105H, V107S, Q739S, R951Q, and H1311Y; S104A, F105H, V107S, P411A, Q413N, and I414V; S104A, F105H, V107S, P411A, Q413N, I414V, R457K, and N459S; T310G and P316D; P411G, Q413A, and I414A; Y430V and F432V; E910S and L911A; and K1123S W1126S.

8. A polynucleotide encoding a tsCas9 according to any of claims 1-7.

9. The polynucleotide according to claim 8, said polynucleotide having at least 80% sequence identity to SEQ ID NO:1.

10. A nucleic acid construct comprising a polynucleotide according to any of claims 8-9.

11. An expression vector comprising a polynucleotide according to any of claims 8-9.

12. A host cell comprising a tsCas9 as defined in any of claims 1-7, a polynucleotide as defined in any of claims 8-9, a nucleic acid construct as defined in claim 10, and/or an expression vector as defined in claim 11.

13. The host cell according to claim 12, said host cell being a eukaryotic or prokaryotic host cell.

14. The host cell according to any of claims 12-13, said host cell being a microorganism host cell selected from the group consisting of bacterial, fungal, yeast, and archeal host cell.

15. The host cell according to any of claims 12-14, said host cell being a bacterial host cell selected from the group consisting of Bacillus, Escherichia, Lactobacillus, Lactococcus Streptococcus, and Streptomyces cell; preferably the host cell is selected from the group consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, Bacillus thuringiensis, Escherichia coli, Lactobacillus acidophilus, Lactobacillus amylovorus, Lactobacillus brevis, Lactobacillus (para)casei, Lactobacillus cellobiosus, Lactobacillus crispatus, Lactobacillus curvatus, Lactobacillus delbrueckii subsp. bulgaricus, L. delbrueckii subsp. lactis, Lactobacillus fermentum, Lactobacillus gallinarum, Lactobacillus gasseri, Lactobacillus helveticus, Lactobacillus johnsonii, Lactobacillus plantarum, Lactobacillus reuteri, Lactobacillus rhamnosus, Lactobacillus salivarius, Lactococcus chungangensis, Lactococcus formosensis, Lactococcus fujiensis, Lactococcus garvieae, Lactococcus lactis, Lactococcus piscium, Lactococcus plantarum, Lactococcus raffinolactis, Lactococcus taiwanensi, Streptococcus equisimilis, Streptococcus pyogenes, Streptococcus uberis, Streptococcus equi subsp. zooepidemicus, Streptomyces achromogenes, Streptomyces avermitilis, Streptomyces coelicolor, Streptomyces griseus, and Streptomyces lividans; more preferably the host cell is Bacillus licheniformis.

16. The host cell according to any of claims 12-15, said host cell being a fungal host cell selected from the group of consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell.

17. The host cell according to any of claims 12-16, said host cell being a yeast host cell selected from the group consisting of Candida, Hansenula, Kluyveromyces, Pichia, Saccharomyces, Schizosaccharomyces, and Yarrowia cell; preferably the host cell is selected from the group consisting of Kluyveromyces lactis, Pichia pastoris, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell.

18. The host cell of any of claims 12-17, wherein the tsCas9 is a nuclease-null variant; preferably said variant comprises an amino acid alteration in a position corresponding to position 10 and position 840 of SEQ ID NO:2; more preferably said variant comprises a substitution of aspartic acid for alanine, D10A, and a substitution of histidine for alanine, H840A.

19. A method of inducing expression of one or more genome target sequence of interest, the method comprising the steps of:
a) providing a host cell according to claim 18, said host cell further comprising one or more suitable gRNA and one or more genome target sequence of interest;
b) cultivating the host cell at a dissociative temperature of the tsCas9, whereby a complex formed in the host cell by the tsCas9 with the one or more suitable gRNA and the one or more genome target sequence of interest disassociates; and subsequently
c) lowering the temperature to a restrictive temperature of the tsCas9 and cultivating the host cell, whereby expression of the one or more target sequence is induced.

20. The method according to claim 19, wherein the dissociative temperature is 42-50° C. and the restrictive temperature is 37-42° C.

21. The method according to any of claims 19-20, said method comprising the following step after providing the host cell and before step (b) of: cultivating the host cell at a permissive temperature of the tsCas9, whereby a complex is formed in the host cell by the tsCas9, the one or more suitable gRNA and the one or more genome target sequence of interest, whereby expression of the one or more genome target sequence is repressed.

22. The method according to any of claims 19-21, said method comprising the additional step of:
d) increasing the temperature to a permissive temperature of the tsCas9, wherein a complex is formed in the host cell by the tsCas9, the one or more suitable gRNA, and the one or more genome target sequence of interest, and cultivating the host cell, whereby expression of the one or more genome target sequence of interest is repressed.

23. The method according to any of claims 21-22, wherein the permissive temperature is 28-34° C.

24. A method of repressing one or more genome target sequence of interest, the method comprising the steps of:
a) providing a host cell according to claim 18, said host cell further comprising one or more suitable gRNA and one or more genome target sequence of interest;
b) cultivating the host cell at a restrictive temperature of the tsCas9, wherein the one or more genome target sequence of interest is expressed; and subsequently
c) lowering the temperature to a permissive temperature of the tsCas9, whereby a complex is formed in the host cell by the tsCas9, the one or more suitable gRNA and the one or more genome target sequence of interest, and whereby expression of the one or more genome target sequence is repressed.

25. The method according to claim 24, wherein the permissive temperature is 28-34° C. and the restrictive temperature is 37-42° C.

26. The method according to any of claims 24-25, said method comprising the following step after providing the host cell and before step (b) of: cultivating the host cell at a dissociative temperature of the tsCas9 to ensure that no complex is formed in the host cell between the tsCas9, the one or more gRNA and the one or more genome target sequence of interest.

27. The method according to any of claims 24-26, said method comprising the additional steps of:
d) raising the temperature to a dissociative temperature of the tsCas9, whereby the complex disassociates; and subsequently
e) lowering the temperature to a restrictive temperature of the tsCas9, whereby expression of the one or more target sequence is induced.

28. The method according to any of claims 26-27, wherein the dissociative temperature is 42-50° C.

29. The method according to any of claims 19-28, wherein the one or more genome target sequence of interest comprises at least 20 nucleotides and further comprises or is flanked by a functional PAM sequence for a Class-II Cas9 protein; preferably the one or more genome target sequence is comprised in an open reading frame encoding a polypeptide or in a promoter region.

30. The method according to any of claims 19-29, wherein the one or more genome target sequence of interest encode one or more enzyme selected from the group consisting of hydrolase, isomerase, ligase, lyase, oxidoreductase, or a transferase; preferably the one or more enzyme is an alpha-amylase, alpha-galactosidase, alpha-glucosidase, aminopeptidase, amylase, asparaginase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, green fluorescent protein, glucano-transferase, glucoamylase, invertase, laccase, lipase, mannosidase, mutanase, oxidase, pectinolytic enzyme, peroxidase, phosphodiesterase, phytase, polyphenoloxidase, proteolytic enzyme, ribonuclease, transglutaminase, and xylanase.

31. The method according to any of claims 19-30, wherein the host cell is a eukaryotic or prokaryotic cell; preferably, the host cell is a microorganism host cell; even more preferably, the host cell is a fungal, bacterial, or archeal cell.

32. The method according to any of claims 19-31 wherein the host cell is a Bacillus host cell; preferably the host cell is selected from the group of Bacillus species consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis; more preferably the host cell is Bacillus licheniformis.

33. The method according to any of claims 19-32, wherein the one or more gRNA comprises a first RNA comprising 20 or more nucleotides that are at least 85% complementary to and capable of hybridizing to the one or more genome target sequence; preferably the 20 or more nucleotides are at least 90%, 95%, 97%, 98%, 99% or even 100% complementary to and capable of hybridizing to the one or more genome target sequence.

34. Use of a tsCas9 according to any of claims 1-7, a polynucleotide according to any of claims 8-9, a nucleic acid construct according to claim 10, an expression vector according to claim 11, a host cell according to any of claims 12-18, and/or a method according to any of claims 19-33 in medicine or cosmetics.

35. Use of a tsCas9 according to any of claims 1-7, a polynucleotide according to any of claims 8-9, a nucleic acid construct according to claim 10, an expression vector according to claim 11, a host cell according to any of claims 12-18, and/or a method according to any of claims 19-33 in medical or biotechnological research or production.

36. Use of a tsCas9 according to any of claims 1-7, a polynucleotide according to any of claims 8-9, a nucleic acid construct according to claim 10, an expression vector according to claim 11, a host cell according to any of claims 12-18, and/or a method according to any of claims 19-33 in genome editing, regulation of gene expression, or CRISPR inhibition.

37. Use of a tsCas9 according to any of claims 1-7, a polynucleotide according to any of claims 8-9, a nucleic acid construct according to claim 10, an expression vector according to claim 11, a host cell according to any of claims 12-18, and/or a method according to any of claims 19-33 in enzyme research, development, and/or production.


SEQUENCE LISTING (9 sequences)

SEQ ID NO: 1 - DNA, 4107 bp, Streptococcus pyogenes
  source 1..4107: mol_type = genomic DNA
atggacaaaaaatacagcatcggcctggatattggcacaaattcagttggctgggcagttatcacagacgaatataaagttccgagcaaaaaatttaaagtcctgggcaatacagatcgccatagcatcaaaaaaaacctgattggcgcactgctgtttgattcaggcgaaacagcagaagcaacaagacttaaaagaacagcaagacgcagatatacaagacgcaaaaatcgcatttgctatctgcaagaaatctttagcaacgaaatggcgaaagtcgacgacagcttttttcatagactggaagaatcatttctggtcgaagaagataaaaaacacgaacgccatccgatttttggcaacattgttgatgaagtcgcgtatcatgaaaaatacccgacaatttatcatctgcgcaaaaaactggttgacagcacagataaagcagatcttcgcctgatttatctggcactggcacatatgatcaaatttagaggccattttctgatcgaaggcgatctgaatccggataattcagatgtcgacaaactgtttattcagctggtccagacatataaccagctgtttgaagaaaatccgattaatgcatcaggcgttgatgcaaaagcaattctgtcagcaagactgtcaaaatcaagacgcctggaaaatctgattgcacaactgcctggcgaaaaaaaaaatggactgtttggcaatcttattgcactgtcactgggcctgacaccgaactttaaatcaaattttgatctggcggaagatgcgaaactgcaactttcaaaagatacgtatgatgacgatctggataatctgctggcgcaaattggcgatcaatatgcagatctttttctggcagcgaaaaatctgtcagatgcaattctgctgtcagatattctgcgcgtcaatacagaaattacaaaagcaccgctgagcgcgagcatgattaaaagatatgatgaacatcatcaggacctgacactgctgaaagcactggttagacaacaactgccggaaaaatacaaagaaatcttttttgatcagagcaaaaacggctatgcaggctatattgatggcggagcatcacaagaagaattttacaaatttatcaaaccgatcctggaaaaaatggatggaacagaagaactgctggttaaactgaatcgcgaagatttactgagaaaacagcgcacatttgataatggctcaattccgcatcaaattcatctgggcgaactgcatgcgattcttagacgccaagaagatttttatccgtttctgaaagacaaccgggaaaaaattgaaaaaatcctgacatttcgcatcccgtattatgtcggaccgctggcaagaggcaattcaagatttgcatggatgacacgcaaaagcgaagaaacaattacaccgtggaattttgaagaagtcgttgataaaggcgcaagcgcacaatcatttattgaacgcatgacgaactttgacaaaaacctgccgaatgaaaaagtcctgccgaaacattcactgctgtatgaatactttacggtctataatgaactgacgaaagtcaaatatgtcacagaaggcatgagaaaaccggcatttctgtcaggcgaacagaaaaaagcgattgtcgatcttctgtttaaaacgaaccgcaaagtcacagtgaaacagctgaaagaagattactttaaaaaaatcgaatgctttgatagcgtcgaaatctcaggcgtcgaagatagatttaatgcaagcctgggcacatatcatgatctgctgaaaatcatcaaagataaagattttctggataacgaagaaaacgaagatatcctggaagatattgtgctgacactgacgctttttgaagatcgcgaaatgattgaagaacgcctgaaaacatatgcgcacctgtttgatgataaagtcatgaaacaacttaaacgcagacgctatacaggctggggcagactttcaagaaaactgattaacggcattcgcgataaacaaagcggcaaaacaatcctggattttctgaaatcagatggctttgcgaatcgcaattttatgcagctgattcatgatgacagcctgacgtttaaagaagatattcagaaagcacaagtttcaggccaaggcgattcactgcatgaacatattgcaaatctggcaggctcaccggcaatcaaaaaaggcattctgcaaacagttaaagtcgtcgatgaactggttaaagttatgggcagacataaaccggaaaacatcgttattgaaatggcacgcgaaaatcagacaacacaaaaaggacagaaaaattcacgcgaacggatgaaaagaattgaagaaggcattaaagaactgggcagccaaatcctgaaagaacatccggttgaaaatacacagctgcagaacgaaaaactgtatctgtattatctgcagaatggacgcgatatgtatgtcgatcaagaactggatattaatcgcctgagcgattatgatgtggatcatattgttccgcagagctttcttaaagatgatagcatcgataacaaagtcctgacacgctcagataaaaacagaggcaaatcagataatgtcccgtcagaagaggttgtcaaaaaaatgaaaaactactggcgtcaactgctgaacgcgaaacttattacacaacgcaaatttgacaatctgacaaaagcagaaagaggcggactgtcagaacttgataaagcgggttttatcaaaagacagctggtcgaaacacgccagattacaaaacatgttgcgcaaattctggatagccgcatgaacacaaaatatgacgaaaacgataaactgatccgggaagtcaaagtcattacgctgaaatcaaaactggtcagcgattttcgcaaagactttcagttttacaaagtccgcgaaatcaacaactaccatcatgcacatgatgcatatctgaatgcagttgtcggcacagcgcttatcaaaaaataccctaaactggaaagcgaatttgtctacggcgactataaagtctatgatgtccgcaaaatgattgcgaaaagcgaacaagaaattggcaaagcgacagcgaaatactttttttacagcaacatcatgaacttttttaaaacggaaatcacactggcgaacggcgaaattagaaaaagaccgcttattgaaacgaacggtgaaacaggcgaaattgtttgggataaaggcagagattttgcaacagttagaaaagttctgagcatgccgcaagtcaacatcgtgaaaaaaacagaagttcagacaggcggatttagcaaagaatcaattcttccgaaacgcaactcagacaaactgattgcgcgtaaaaaagactgggacccgaaaaaatacggtggctttgattcaccgacagttgcatattcagttctggttgttgcgaaagtggaaaaaggcaaatccaaaaaacttaaaagcgtgaaagaacttctgggcatcacaattatggaacgctcgagctttgaaaaaaacccgatcgactttctggaagccaaaggctataaagaagtgaaaaaagaccttattatcaaactgccgaaatacagcctgtttgaactggaaaatggcagaaaacgcatgctggcatcagcaggcgaacttcagaaaggcaatgaactggcactgccgtcaaaatatgttaactttctgtatctggcgagccattacgaaaaacttaaaggctcaccggaagataacgaacagaaacaactgtttgtcgaacagcataaacattacctggacgaaatcatcgaacaaatcagcgaattttcaaaacgcgttattctggcagatgcgaacctggataaagttcttagcgcatataacaaacaccgggataaaccgattagagaacaagcggaaaatatcattcacctgtttacactgacaaatcttggcgcaccggcagcgtttaaatactttgatacaacaattgaccgcaaacgctacacaagcacaaaagaagttctggacgcaacactgattcatcaatcaattacaggcctttatgaaacgcgcattgatctgtcacaactgggaggcgattga

SEQ ID NO: 2 - protein, 1368 aa, Streptococcus pyogenes [sequence omitted]

SEQ ID NO: 3 - DNA, 9936 bp, synthetic construct
  misc_feature 1..9936: note = MOL2212-amyL locus
  source 1..9936: mol_type = other DNA
tctagaccttctttgtgcttggaagcagagcccaatattatcccgaaacgataaaacggatgctgaaggaaggaaacgaagtcggcaaccattcctgggaccatccgttattgacaaggctgtcaaacgaaaaagcgtatcaggagattaacgacacgcaagaaatgatcgaaaaaatcagcggacacctgcctgtacacttgcgtcctccatacggcgggatcaatgattccgtccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgttcgcagatgctgctgaagagattattaaaaagctgaaagcaaaaggctatcaattggtaactgtatctcagcgcatgcagcaggtagttctatcaaaccgtgaattgacagtatatagagaagatatttctcgcctggctgatcaagaacagcagacgctgattgatgcttttatgactaaggatcgggaaaaaggatttgatttgcaaaaagatcctttaatgcgtcttgccctttttgatagaggagacagccaatatacatgtgtctggacacatcaccatatcatcatggatgggtggtgtctcggcattattcttaaagagtttttcagcatgtatgattcgctcaaaaataactcacctgtacagcttggcagcacggtgccgtacagccgttatattgaatggctcggagaacaagatcaagaagagactgctgcctactggagcgaatatttgaaggagtacggcaatactgcttctattccccgaataaagcgccgcacggcagacgggaattataaagccgaccaggtcagcttttcattagcgccggatatggttgagaaactgacagaggctgcccaaaactggggagtgacattaaacacgctgtttatgagtatttggggcgtgctgctccatcgatataatgctgcagatgatgccgtttttggctccgtcatttcgggacgcccgtcagcgattgacgggattgaatcaatggtcggtttgtttatcaatactgttccagtgcgaatccgatctgcggaaggcataacattttcttcgctggtcaaagccgtgcaggaggatattttatcatctgaacagcatgggtattatccgctttatgagattcaaaaccatagtccattgaagcaagggttgatcgatcatatttttgtttttgaaaattaccctgtacagcttcatcaggcattaagcgtggagagtgaaaatgacgaaggcgctctgaagctgagtgacatttcaatgtcggaacaaaccaattacgactttaatatcgttatcgttccgggagaatcattttatatcaaattcagctacaacgcagacgtttacgagcgggaggagatgctgaggattcaggggcatctaaagcaagcactggattgcattttgacaaatcccgatgttgccgtcagtgacatcaatatcgtgccgccggaggagcaacaggttatccaattgtttaacgaaactgagcgtccttatgtgaacaaaacgataccccaattgtttgaggaacaagctcacaaaacacctgaagctgctgcattgaaaatgggaaacgagtgctggacctatcgccagttacaagtaagagccaatcagattgcgcacgccctgatagaaaaaggcgttggatctggggatatcgttgctgtaatgatgggccggtcaatggaaatgcctgcagctctgcttgggatttggaaagcaggcggcgcttatatgccgcttgatccacattttccagcagaacgtctttcttttcttctgaaggacagtcaagcggctcaattgctgatagaagaagaccttatttccttgatccctccgtcctatgaaggaaatacgataacgatagaacatacagaaagttaccaaacagaagcgccaaatatgccgccaggtgatctagcctacttgatctatacgtcgggaacgacggggcgccctaaaggggttttagttgatcatcacggtattgccaatacattgcaatggagacgggaagagtatagcatgaccgaacaggatatatccctccatttgttttcgtacgtgtttgacggctgtgtaacgagcttatttaccccgcttttatctggtgcgtgcgtactgctgacaacagatgacgaagcgaaggatgtgctggcccttaagcgaaaaatagcccgttataaggtcagccacatgatcattgttccttccctgtacagggtgttattggaagtgatgactgctgacgatgcaaaaagtcttcgtattgtgacatttgcgggtgaagcggtcacgcctgatctgcttgagttgaatcaaataatttgtccttctgctgaactggcaaatgaatacgggcccacagaaaacagtgtggcaacaacaatattgcggcatctgaataaaaaagagaggatcacgatcggacacccgatcagaaacacaaaagtatttgttttgcacggaaatcaaatgcagccgatcggcgcggcgggtgaactgtgtatttccggcgcgggtcttgcgagaggatactacaaacagcaagagctgacacagaaagcattctccgatcatccattccttgaaggggagcgtttataccgaacaggtgatgcaggccgctttttgcctgatggaacgattgaatatattggacgttttgatgatcaagtgaaaattaggggttatcgtattgaactgagagagattgaaacagttcttcgacaagcaccgggggtaaaagaagcagcggtactggcccgtgatgtttctgctgaggaaaaggagctcgttgcttatatcgttccggaaaagggaaacagcctcccagatttgtatcagcatcttgccgggacattgccgtcctatatgatcccagcaagtattatcaacatcagccagatgccattaacatccagcggcagatcacccgcgataccgtcattttcgacacatttctttctttgctacatcagataacgttgccatttcatccccgccttacctagggattctaaactgtcagcaatattcctgagggcttatgacactttgttaaaattaattataaaatgtaatcaacgaaatttataagacgggcaaaataaaaaaacggatttccttcaggaaatccgtcctctctgctcttctatctttgaacataaattgaaaccgacccgccgtttacgtgaaactctccccagccttccgaattgatgacaaccggctccgaacggtttccggtaatgtcatgccatgtctcaccggcgttttgccggccgacatacattcgctttgccccaccgggtccgtctgttattaatgccgccaaacctgaatttgcaaccgagctgtcgccttcccttgtccagccgacaatgtcatggtggtcgaaataatcatgctgtgctccgtacgcatactgttttctcgcttttaagatcggttcaattttgtgtttcaaggcaggaatttcgcgctgggagtctcctttcgtcccgtacatatccccgtagaaaacctgagggtatccagattcccttgtgagaataaaagcgtaagcaagcggcttaaaccatgtttggacagtcgactcaagcgattgccccggctgtgtatcatggttatcgacaaatgtaaccgatttcaacggatgcttggaaacgaccgtacccttcagcaatttcctcatatcatagccgcctccctgtgtcgatgcagcatggaactgataatgaagcggcacgtcaaacactgaatgattaaaatttgttttgttcaaatagttttccagcgcgcccaagtcattctgccaatattcagctaccgtaaacatttccttccccgttttttccctgacatgattaacccaatcccgcaaaaaagaaaatttaatgtgtttgacagcatcaagacggtttccgtccaattgcagttcattggcataccaagtgccccatctcttaatttctgctgcgacatcaggatggtcataatcgatgtcggcatacatcaaataatcatagttgccgttttcattggaaacttcccaatcccaagcctttccttgaaacttatagatgcggttcagctttcgggactcgtcccaatcggttccgtcaaaatggtaccaatgccatttaaaatcgctgtatgtgctgccggcccccggaaaatgaaaatgtgtccaggctttaattaggtgttctcctgagattacgcggttgcggtcagcgggatcgacttcaaccgcggttacatcttcggtcgcatcagcgccgcctttgtggttgatgaccacatccccgtaaacgttaatgtcgcgggaatgaagacttttgatcgcagattgcagctctccttttgtgccgtactttgtccgaaccgtccctttttgatgaaactcccctaaatcataaaggtcgtaagcaccgtagcccacatccgcttggctcgttcccttatatgccgggggaatccagacggcagtaataccgtgttcagccaaatatgccgagtcgttttgcaaacgcttccaatgttggccgtcattgggcatgtaccattcaaaatactgcatcagcgtcccattaagatttgccgccgctgctgcagaatgaggcagcaagaagatgagcgcaaataacagcgtcagcaatcgggcgtaaagccgtttttgttgtttcatgattctcctcccctttcaatgtgatacatatgatattgtataaatattccgaatttttaacaagtaccattttccctatattttcttccaagatctgtcgaccgcggctagagcggccgcacgcgtgctagccatggcttttacaatagaaggaaaagtcaccccgcagttttttcaaaacagcatcaacgctcttgtagaaagacatgatattttcagaacgatttttatcagccaaaatgtttcctccccccagcaggttgttctgagagaacgaaatgtcatcgtactggaagaagacattactcatttaaacgaggcggagcaatcacagtttatcgagcaatggaaagaaaaggaccgggaccgggggtttcatttgcaaaaggatgtgctcatgcgcatcgctctgattcaaacaggagagagccagtactcttgcatttggacctttcatcatatcatgatggacggttggtgcctcagcattgtgctaaaggagtttctgcatatttacgcatcttatgtaaatgcatccccaataacattggagccggtccagccatacggaaagtatatcaaatggctgatggagcaagacaaagagcaggcggtttcttattgggatcattatctttccggccacgaacagcaaaccgtgctcccgaagcagaaaaaaacaaagggaaaaagcaggcaggaacacgttacattttcattctctaaggaagaaagcagcaggctttcagagcttgctgctagagaagaagtgacactcagtacgatttttcataccatttggggcattctgctgcaaaaatataataataacgatgacgccgtattcggttctgtcatttcaggaagacccgcagaaatcgaaggcattgaacatatggtaggtcttttcattaatacgatgccggttcgggtgcagggggcaaaaacgccttttcttcaacttataaaagatatgcaaaaagaccgtcttgccgctgaagcgtacagctaccaccctttatatgagattcaatcccgctcagctgttaagcaagggctgattgatcatattctcgtatttgaaaactatccggtgcagcaagagattcaaatgctgaacaaacaggaacatgcttctgatctttttcagattcataatttcactgtcgcagatgaaaccaactacagcttttatcttatggtggcgccaggggaagaaattcatatcaaaatgaactatgatgcggagcagcacgatcggtcttttgtgctctctgtcaaagagcatcttctgaatgctgtatcacaaatcttaaataatccgaatcttccacctgaagaaatcgatatcacgacagatactgaaaagcggcagctgattggagaaatcacagatcaaacaccagtttacgaaaccatccatgccatgtttgaaaagcaagcggaaaagacaccagatgctcatgctgtaattgatcaagcctgctcattaacatacagagaactgaataaagcggctaacagattggcacggcatttacgaatgaaaggcgttgtgagacaggaacccgttgcgattatgatggaacgctcagcggcgtttatcacaggtgttcttggtatcttgaaagcaggcggcgccattgttccggttgatccgcattaccctgctgacagaatccgttatattctgcatgattgcggctgttcgcatgttgtttcacaagcgcatcttccctcatcgttagaagacaattacattatcactcatccagaagatatcgaaagcaaagtagacggcagtaacataaagtcagtcaacaatgctgacgatctgctgtatatgatttatacatcaggcacaacaggtaaaccaaagggtgttcaatttgagcatcgaaacatggctaacttgctgaagttcgaatatactcactccggcattgactttgaagcagatgttctgcaatttgcgacgccttccttcgacgtctgctatcaagaaatattttctgcgcttctgaaaggcggcacactccacatcgtgccagaagccataaaaagagatgtgcctcagctgtttgcatttataaacaagcatcagacgaatattgtgtttctcccaactgcttttatcaaaatgattttcagcgagcgagaacttgcgaactcgtttcctgatggcgtcaaacacctgatcgcagccggggaacaattaatgatttcagaccttttccaagatgtgttgcgtaaacgcggcatacacctgcacaatcattacggaccttcagaaactcatgtggtatctacgtataccattcatcctggagaccctattcctgagcttccgccaatcggtaaaccgataggctgtactgatctatatattttgaatcatcaaaaacagcttcagccatgcggcgtaccaggcgaactctatatttccggcgcaagtgttgccagaggatatgtcaatcacgataaactgacaagcgacaaattttcatccgatccattcaagcctgatgtgatcatgtaccggacaggtgatttggccagacggcttgaagacggcaacatcgaatatatcggacgggcagacaatcaagtgaaaatcagaggttatcgaattgagccgcaagaaatcgaagtcacgctaatgaatcatccggacattagtgaggctgccattctcatctggcaagatcaaaacggtgaacatgaattatgcgcttactattgcagtgtacaaaagctgaacacaatagatttacgcagttatatggctagtgagctcccagaatacatgattccggcgaaatggatatgggttgacagcatacctcttaccccaaacggaaaagtagatcgcgcagcacttccggagccagatgcttcaatcagcggaaacccatatactgcccctcgaaatctgttagaggccatggctagcacgcgtgcggccgccgcggtcgacagatcttggaagaaaatatagggaaaatggtacttgttaaaaattcggaatatttatacaatatcatatgtatcacattgaaaggggaggagaatcatgaaacaacaaaaacggctttacgcccgattgctgacgctgttatttgcgctcatcttcttgctgcctcattctgcagcagcggcggcaaatcttaatgggacgctgatgcagtattttgaatggtacatgcccaatgacggccaacattggaagcgtttgcaaaacgactcggcatatttggctgaacacggtattactgccgtctggattcccccggcatataagggaacgagccaagcggatgtgggctacggtgcttacgacctttatgatttaggggagtttcatcaaaaagggacggttcggacaaagtacggcacaaaaggagagctgcaatctgcgatcaaaagtcttcattcccgcgacattaacgtttacggggatgtggtcatcaaccacaaaggcggcgctgatgcgaccgaagatgtaaccgcggttgaagtcgatcccgctgaccgcaaccgcgtaatctcaggagaacacctaattaaagcctggacacattttcattttccgggggccggcagcacatacagcgattttaaatggcattggtaccattttgacggaaccgattgggacgagtcccgaaagctgaaccgcatctataagtttcaaggaaaggcttgggattgggaagtttccaatgaaaacggcaactatgattatttgatgtatgccgacatcgattatgaccatcctgatgtcgcagcagaaattaagagatggggcacttggtatgccaatgaactgcaattggacggaaaccgtcttgatgctgtcaaacacattaaattttcttttttgcgggattgggttaatcatgtcagggaaaaaacggggaaggaaatgtttacggtagctgaatattggcagaatgacttgggcgcgctggaaaactatttgaacaaaacaaattttaatcattcagtgtttgacgtgccgcttcattatcagttccatgctgcatcgacacagggaggcggctatgatatgaggaaattgctgaagggtacggtcgtttccaagcatccgttgaaatcggttacatttgtcgataaccatgatacacagccggggcaatcgcttgagtcgactgtccaaacatggtttaagccgcttgcttacgcttttattctcacaagggaatctggataccctcaggttttctacggggatatgtacgggacgaaaggagactcccagcgcgaaattcctgccttgaaacacaaaattgaaccgatcttaaaagcgagaaaacagtatgcgtacggagcacagcatgattatttcgaccaccatgacattgtcggctggacaagggaaggcgacagctcggttgcaaattcaggtttggcggcattaataacagacggacccggtggggcaaagcgaatgtatgtcggccggcaaaacgccggtgagacatggcatgacattaccggaaaccgttcggagccggttgtcatcaattcggaaggctggggagagtttcacgtaaacggcgggtcggtttcaatttatgttcaaagatagaagagcagagaggacggatttcctgaaggaaatccgtttttttattttgcccgtcttataaatttctttgattacattttataattaattttaacaaagtgtcatcagccctcaggaaggacttgctgacagtttgaatcgcataggtaaggcggggatgaaatggcaacgttatctgatgtagcaaagaaagcaaatgtgtcgaaaatgacggtatcgcgggtgatcaatcatcctgagactgtgacggatgaattgaaaaagcttgttcattccgcaatgaaggagctcaattatataccgaactatgcagcaagagcgctcgttcaaaacagaacacaggtcgtcaagctgctcatactggaagaaatggatacaacagaaccttattatatgaatctgttaacgggaatcagccgcgagctggaccgtcatcattatgctttgcagcttgtcacaaggaaatctctcaatatcggccagtgcgacggcattattgc

SEQ ID NO: 4 - DNA, 3276 bp, synthetic construct
  misc_feature 1..3276: note = MOL2212-xyl locus
  unsure 3139
  source 1..3276: mol_type = other DNA
gaattcaaaagccgcttccgccctggctttcgctttatccaaaggatgtgtcagccggttccacgcccggaacatcgtcccgtcgccgaaagggtctttcccgtccgcatcaaaggtatgccaatacgcaacagcgaagcgaaggtgttccttcatcgttttgccgccgacaaattcatcagggttgtaatatttaaatgcgtaaggattttccgaatcggccccttcatactcaatcattccgatatttctaaaaaacattccgatctcccccttcacttttccttgcaaacgttaaaaaacaatgtttgtttaaccattaaactaacttccttgtatgtattttacaggatcaattaatcgctttcaatggaaatagccgcggatcgatgctagcagatcttggaagaaaatatagggaaaatggtacttgttaaaaattcggaatatttatacaatatcatatgtatcacattgaaaggggaggagaatcatgaaacaacaaaaacggctttacgcccgattgctgacgctgttatttgcgctcatcttcttgctgcctcattctgcagcagcggcggcaaatcttaatgggacgctgatgcagtattttgaatggtacatgcccaatgacggccaacattggaagcgtttgcaaaacgactcggcatatttggctgaacacggtattactgccgtctggattcccccggcatataagggaacgagccaagcggatgtgggctacggtgcttacgacctttatgatttaggggagtttcatcaaaaagggacggttcggacaaagtacggcacaaaaggagagctgcaatctgcgatcaaaagtcttcattcccgcgacattaacgtttacggggatgtggtcatcaaccacaaaggcggcgctgatgcgaccgaagatgtaaccgcggttgaagtcgatcccgctgaccgcaaccgcgtaatctcaggagaacacctaattaaagcctggacacattttcattttccgggggccggcagcacatacagcgattttaaatggcattggtaccattttgacggaaccgattgggacgagtcccgaaagctgaaccgcatctataagtttcaaggaaaggcttgggattgggaagtttccaatgaaaacggcaactatgattatttgatgtatgccgacatcgattatgaccatcctgatgtcgcagcagaaattaagagatggggcacttggtatgccaatgaactgcaattggacggaaaccgtcttgatgctgtcaaacacattaaattttcttttttgcgggattgggttaatcatgtcagggaaaaaacggggaaggaaatgtttacggtagctgaatattggcagaatgacttgggcgcgctggaaaactatttgaacaaaacaaattttaatcattcagtgtttgacgtgccgcttcattatcagttccatgctgcatcgacacagggaggcggctatgatatgaggaaattgctgaagggtacggtcgtttccaagcatccgttgaaatcggttacatttgtcgataaccatgatacacagccggggcaatcgcttgagtcgactgtccaaacatggtttaagccgcttgcttacgcttttattctcacaagggaatctggataccctcaggttttctacggggatatgtacgggacgaaaggagactcccagcgcgaaattcctgccttgaaacacaaaattgaaccgatcttaaaagcgagaaaacagtatgcgtacggagcacagcatgattatttcgaccaccatgacattgtcggctggacaagggaaggcgacagctcggttgcaaattcaggtttggcggcattaataacagacggacccggtggggcaaagcgaatgtatgtcggccggcaaaacgccggtgagacatggcatgacattaccggaaaccgttcggagccggttgtcatcaattcggaaggctggggagagtttcacgtaaacggcgggtcggtttcaatttatgttcaaagatagaagagcagagaggacggatttcctgaaggaaatccgtttttttattttgcccgtcttataaatttcgttgattacattttataattaattttaacaaagtgtcataagccctcaggaatattgctgacagtttagaatccctaggtaaggcggggatgaaatggcaacgttatctgatgtagcaaagaaagaaatgtgtcgaaaatgacggtatcgcgggtgatctgcggccgcacgcgtgtcgacagtactgaaatagaggaaaaaataagttttcaaaatttgttatactataataaattcacagcttcaaagcaatggagtgattgacattgaatacagcggatcaagcccttgtaaaaaaaatgaacaaagccctgatttttgagcagattattgaaaacggacccgtctcaagggccaaactttcggaaataacaggattaaataaagcgaccgtttcctcccaagtgtcttccctgcttcaaaaggacatcatttatgaaaccggccccggcgagtcaagcggcggcagaaggcccgttatgctgaaattcaatcgaaaagccggctatgcggtcggtgtagatgtcggaaccaattatataattgttgcgttaaccgaccttgaagggcatttgattgaacaattcgaacgaacgcttgatgaagaagacattcaggcgacagaagaggcattaatcgaattgacggggcttgccgttgacaaaataccgccttccccattcggattgaccggaatcggcgtgtgcgtgcccggcttggtggacaacgaacggcatgtagtctttacgccaaataagccgatccatttgattccgataaaagaaaaactggaagaaaggttcggcgttcccattttaattgaaaacgaagccaacgccggcgccgttggcgagaaggaatacggggaaggcggccagcttgagcatgccgtgtttgtcagcattaataccgggatcggtttagggattttaatgaatggaaaattgtttagaggtgtgcaaggtttttccggagaagccgggcacatgtccattcactttgacggtccgttatgccggtgcggaaatagaggctgctggnagctctacgcttcagaaaaagccgtcttctctcactatgcagaagcttgcatgcctgcaggtcgattcacaaaaaataggcacacgaaaaacaagttaagggatgcagtttatgcatcccttaacttacttattaaa

SEQ ID NO: 5 - DNA, 3521 bp, synthetic construct
  misc_feature 1..3521: note = MOL2212-gnt locus
  source 1..3521: mol_type = other DNA
gcagcggttcgtttaatcgggaatgcctcgacttcgatggtcatctccctgttggtcgcgatctatacgatggggatcgccagaaagatcccgatcaaacaagtgatggattcctgttcaaccgccattacacaaatcggaatgatgctcttgatcatcgggggcggcggcgccttcaaacaagtcttgatcaatggcggagtaggcgactatgtagctgaattattcaaaggaacagccatgtcgccgatcttgctcgcctgggtcatcgccgcaattctgcgcatctccttaggatccgcgacagttgctgcattaagcacaaccggactcgttcttccgatgctcggacaaagcgatgtcaatcttgcgctggttgtgcttgcaacaggggccggaagtgtaatcgcttcccatgtcaatgacgcaggcttctggatgttcaaagagtatttcggactaagcatgaaagagacatttgccacctggacactgcttgaaaccattatcgcggtggccggattaggatttactttattgttaagcctatttgtataaaaaagatcacccgcgataccgtcattttcgacacatttctttctttgctacatcagataacgttgccatttcatccccgccttacctagggattctaaactgtcagcaatattcctgagggcttatgacactttgttaaaattaattataaaatgtaatcaacgaaatttataagacgggcaaaataaaaaaacggatttccttcaggaaatccgtcctctctgctcttctatctttgaacataaattgaaaccgacccgccgtttacgtgaaactctccccagccttccgaattgatgacaaccggctccgaacggtttccggtaatgtcatgccatgtctcaccggcgttttgccggccgacatacattcgctttgccccaccgggtccgtctgttattaatgccgccaaacctgaatttgcaaccgagctgtcgccttcccttgtccagccgacaatgtcatggtggtcgaaataatcatgctgtgctccgtacgcatactgttttctcgcttttaagatcggttcaattttgtgtttcaaggcaggaatttcgcgctgggagtctcctttcgtcccgtacatatccccgtagaaaacctgagggtatccagattcccttgtgagaataaaagcgtaagcaagcggcttaaaccatgtttggacagtcgactcaagcgattgccccggctgtgtatcatggttatcgacaaatgtaaccgatttcaacggatgcttggaaacgaccgtacccttcagcaatttcctcatatcatagccgcctccctgtgtcgatgcagcatggaactgataatgaagcggcacgtcaaacactgaatgattaaaatttgttttgttcaaatagttttccagcgcgcccaagtcattctgccaatattcagctaccgtaaacatttccttccccgttttttccctgacatgattaacccaatcccgcaaaaaagaaaatttaatgtgtttgacagcatcaagacggtttccgtccaattgcagttcattggcataccaagtgccccatctcttaatttctgctgcgacatcaggatggtcataatcgatgtcggcatacatcaaataatcatagttgccgttttcattggaaacttcccaatcccaagcctttccttgaaacttatagatgcggttcagctttcgggactcgtcccaatcggttccgtcaaaatggtaccaatgccatttaaaatcgctgtatgtgctgccggcccccggaaaatgaaaatgtgtccaggctttaattaggtgttctcctgagattacgcggttgcggtcagcgggatcgacttcaaccgcggttacatcttcggtcgcatcagcgccgcctttgtggttgatgaccacatccccgtaaacgttaatgtcgcgggaatgaagacttttgatcgcagattgcagctctccttttgtgccgtactttgtccgaaccgtccctttttgatgaaactcccctaaatcataaaggtcgtaagcaccgtagcccacatccgcttggctcgttcccttatatgccgggggaatccagacggcagtaataccgtgttcagccaaatatgccgagtcgttttgcaaacgcttccaatgttggccgtcattgggcatgtaccattcaaaatactgcatcagcgtcccattaagatttgccgccgctgctgcagaatgaggcagcaagaagatgagcgcaaataacagcgtcagcaatcgggcgtaaagccgtttttgttgtttcatgattctcctcccctttcaatgtgatacatatgatattgtataaatattccgaatttttaacaagtaccattttccctatattttcttccaagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctctaataattgttaatcatgttggagctcagtgagagcgaagcgaacacttgattttttaattttctatcttttataggtcattagagtatacttatttgtcctataaactatttagcagcataatagatttattgaataggtcatttaagttgagcatattagaggaggaaaatcttggagaaatatttgaagaacccgagaaaaatctttcttatggtcacagcaggcaaaccggttgattctgttatcgactcgctcgttcctctgcttgaagaaggggatgtcatcatggacggcggcaactcccactacgaagatacggaacgaagatatgacagtttgaaagcgaaaggtatcggctacttgggaatcggcatttccggcggcgaagtcggcgctttaaaaggcccatccattatgcccggcggagatcgggacgtctacgaaaaagcagctccgattctcacaaaaatcgccgcccaagtagaaggagatccatgctgtgtctacatcggtccaaaaggcgcggggcattttgttaaaatggtgcacaacggcatcgaatacgcagacatgcagcttatcgctgaagcgtatacatttttaagagaaaagcttcttttgccgatagatgaaatcgctgacattttcgacacgtggaatcaaggagagctgaaaagctatttaatcgaaatcacggcagagatcctgaggaaaaaggatgagcgaacgggcgctccgctcatcgacgtcatcctcgacaaaaccggccaaaaaggcacgggcaaatggacgagcctgcaggccatcgacaacggcattccatcatcaattatcacggaatccctgtttgcccgttacctgtcatcattaaaagacgaacggacagctgccgaaaacgtattggccgggcctgaaacagaagaac

SEQ ID NO: 6 - DNA, 2323 bp, synthetic construct
  misc_feature 1..2323: note = MOL2212-prsA locus
  unsure 1005
  unsure 1390
  source 1..2323: mol_type = other DNA
aattcattccgccttttgaagcggagtagtgaacatttttcggtctcggaatttgctgatgaacgctggatatattgagtatgcttcctttcatgcggcggtccgccatatattttaaggcttcgcgtgcgcagagaaacgcaccggttaaattaacgtcgatcactttctgccactcatcaagctcaagttcgtgcggaaaggcgcccgcgccgttaaatcctgaattattgaccataacatcaagtgaaccaaagtgttcaactgctgaattaatcagctttttcacgtcctcttcggaagacacgtcagcttctatcgcaagggcgtttccgcccgctgcttgaatattgtcgacggcttcccgtgcgcctccgggatcgctgtgataattgacaaccacattcatcttttcttttccaaaccgttctgcaaccgccttgcctataccttttgaagagccggtcacaattgctgtttttccttttaaatcactatacaacctaaacacccctcaatttcttttctccatgtacattacccggtatcagctagcagatccatgagtagaagcgccatatcggcgcttttcttttggaagaaaatatagggaaaatggtacttgttaaaaattcggaatatttatacaatatcatatgttacacattgaaaggggaggagaatcatgaaacaacaaaaacggctttacgcccgattgtgaattcataattcccctctgttgaaaatctttttacagcatgtcagaatatgatatgatacaattcaaaggaaagtttaaactgttatgattaggagtgtttgcatttatgaagaagattgcaattgcggcgattacagcgacaagcgtgctggctctcagcgcatgcagcgggggagattctgaggttgttgcggaaacaaaagctggaaatattacaaaagaagacctttatcaaacattaaaagacaatgccggagcggacgcactgaacatgcttgttcagnaaaaagtactcgatgataaatacgatgtctccgacaaagaaatcgacaaaaagctgaacgagtacaaaaaatcaatgggtgaccagctcaaccagctcattgaccaaaaaggcgaagacttcgtcaaagaacagatcaaatacgaacttctgatgcaaaaagccgcaaaggataacataaaagtaaccgatgatgacgtaaaagaatattatgacggcctgaaaggcaaaatccacttaagccacattcttgtgaaagaaaagaaaacggctgaagaagttgagaaaaagctgaaaaaaggcgaaaaattcgaagaccttgcaaaagagtattcaactgacggtacagccgaaaaaggcggcgacctcggctgggtcggcaaagncgataacatggacaaggatttcgtcaaagcggcatttgctttgaaaaccggcgaaatcagcggacctgtgaaatcccaattcggctatcacatcattaaaaaagacgaagaacgcggcaaatatgaagacatgaaaaaagagcttaaaaaagaagtccaagaacaaaagcaaaatgatcaaactgaactgcaatccgtcattgacaaacttgtcaaagatgctgatttaaaagtaaaagacaaagagttgaaaaaacaagtcgaccagcgtcaagctcagacaagcagcagcagctgacgccaaaaaagctgtcctcccctcgttggggtcggacagctttttttatgcgatggaatggctgtcagctgcaggtacccgggtctagagtcgacgcggccgtatacttaatttcctttaagcctgtactttttgccatctattgatatcgtgaaatttgaaggaccgctgatcggcaaataatagacaagctgaaactccgcttcctcaccaggtttaatggttttccagcttgatagagtcactctgatgcggtggaaatctcctgtcagtccgccaatgttcggtcccgtatgccctttagaaatcacttcaacatgatcgccgctccagcttctaaaccgaggggaagtggatgtcggcgcatcaaattcgataaaagaacctcccgtaatcgtgacatcgctgttgttgatcagcttcatcaccgggtggatcgggtaattttgatctcctaacggaaagttcgtcaactctacttctgcatctagtgattcatcaggcaaaggtgatgtgctgagtcgattatcgtatggtgttgatccgagaatttatgatactcgttctcgttaaggtattgccgataccgtattctcctttactgccgtttcgttcgggataccagtcataatcag

SEQ ID NO: 7 - DNA, 6165 bp, synthetic construct
  misc_feature 1..6165: note = forD:cas9d-gDNA(p4199)-cat locus
  source 1..6165: mol_type = other DNA
cggatcaaatgatcttcaggagtcagcataatacatccagttcaggtagataagatttgaatttggtgacttgcttttgttcttcttctttcattttctgactaatccaaactggaaaaagcaggtcttttaacagattaggaggtttctgacatgcaccattcggtcactaaccgaatgcagtaaaggacactgtggtgcttgccagccattagggtattgaggaggtgatcaaaatgctaggtgacagtatttcgtcgaagtggacaagtcgtgaccaaatgacctcggatcgagggttggtcatggaggaaaaaattgatgtctggtgacaaagaggagtcatgatcatggcaccgccaacgagggaaaaaactcttcccgcatcgacacggtatgtgggcggtgacaaactaacttatagagtaaatttattagtcgaatgaaagacgcgctaaaaatgaggagggaagcgaatggacaaaaaatacagcatcggcctggctattggcacaaattcagttggctgggcagttatcacagacgaatataaagttccgagcaaaaaatttaaagtcctgggcaatacagatcgccatagcatcaaaaaaaacctgattggcgcactgctgtttgattcaggcgaaacagcagaagcaacaagacttaaaagaacagcaagacgcagatatacaagacgcaaaaatcgcatttgctatctgcaagaaatctttagcaacgaaatggcgaaagtcgacgacagcttttttcatagactggaagaatcatttctggtcgaagaagataaaaaacacgaacgccatccgatttttggcaacattgttgatgaagtcgcgtatcatgaaaaatacccgacaatttatcatctgcgcaaaaaactggttgacagcacagataaagcagatcttcgcctgatttatctggcactggcacatatgatcaaatttagaggccattttctgatcgaaggcgatctgaatccggataattcagatgtcgacaaactgtttattcagctggtccagacatataaccagctgtttgaagaaaatccgattaatgcatcaggcgttgatgcaaaagcaattctgtcagcaagactgtcaaaatcaagacgcctggaaaatctgattgcacaactgcctggcgaaaaaaaaaatggactgtttggcaatcttattgcactgtcactgggcctgacaccgaactttaaatcaaattttgatctggcggaagatgcgaaactgcaactttcaaaagatacgtatgatgacgatctggataatctgctggcgcaaattggcgatcaatatgcagatctttttctggcagcgaaaaatctgtcagatgcaattctgctgtcagatattctgcgcgtcaatacagaaattacaaaagcaccgctgagcgcgagcatgattaaaagatatgatgaacatcatcaggacctgacactgctgaaagcactggttagacaacaactgccggaaaaatacaaagaaatcttttttgatcagagcaaaaacggctatgcaggctatattgatggcggagcatcacaagaagaattttacaaatttatcaaaccgatcctggaaaaaatggatggaacagaagaactgctggttaaactgaatcgcgaagatttactgagaaaacagcgcacatttgataatggctcaattccgcatcaaattcatctgggcgaactgcatgcgattcttagacgccaagaagatttttatccgtttctgaaagacaaccgggaaaaaattgaaaaaatcctgacatttcgcatcccgtattatgtcggaccgctggcaagaggcaattcaagatttgcatggatgacacgcaaaagcgaagaaacaattacaccgtggaattttgaagaagtcgttgataaaggcgcaagcgcacaatcatttattgaacgcatgacgaactttgacaaaaacctgccgaatgaaaaagtcctgccgaaacattcactgctgtatgaatactttacggtctataatgaactgacgaaagtcaaatatgtcacagaaggcatgagaaaaccggcatttctgtcaggcgaacagaaaaaagcgattgtcgatcttctgtttaaaacgaaccgcaaagtcacagtgaaacagctgaaagaagattactttaaaaaaatcgaatgctttgatagcgtcgaaatctcaggcgtcgaagatagatttaatgcaagcctgggcacatatcatgatctgctgaaaatcatcaaagataaagattttctggataacgaagaaaacgaagatatcctggaagatattgtgctgacactgacgctttttgaagatcgcgaaatgattgaagaacgcctgaaaacatatgcgcacctgtttgatgataaagtcatgaaacaacttaaacgcagacgctatacaggctggggcagactttcaagaaaactgattaacggcattcgcgataaacaaagcggcaaaacaatcctggattttctgaaatcagatggctttgcgaatcgcaattttatgcagctgattcatgatgacagcctgacgtttaaagaagatattcagaaagcacaagtttcaggccaaggcgattcactgcatgaacatattgcaaatctggcaggctcaccggcaatcaaaaaaggcattctgcaaacagttaaagtcgtcgatgaactggttaaagttatgggcagacataaaccggaaaacatcgttattgaaatggcacgcgaaaatcagacaacacaaaaaggacagaaaaattcacgcgaacggatgaaaagaattgaagaaggcattaaagaactgggcagccaaatcctgaaagaacatccggttgaaaatacacagctgcagaacgaaaaactgtatctgtattatctgcagaatggacgcgatatgtatgtcgatcaagaactggatattaatcgcctgagcgattatgatgtggatgctattgttccgcagagctttcttaaagatgatagcatcgataacaaagtcctgacacgctcagataaaaacagaggcaaatcagataatgtcccgtcagaagaggttgtcaaaaaaatgaaaaactactggcgtcaactgctgaacgcgaaacttattacacaacgcaaatttgacaatctgacaaaagcagaaagaggcggactgtcagaacttgataaagcgggttttatcaaaagacagctggtcgaaacacgccagattacaaaacatgttgcgcaaattctggatagccgcatgaacacaaaatatgacgaaaacgataaactgatccgggaagtcaaagtcattacgctgaaatcaaaactggtcagcgattttcgcaaagactttcagttttacaaagtccgcgaaatcaacaactaccatcatgcacatgatgcatatctgaatgcagttgtcggcacagcgcttatcaaaaaataccctaaactggaaagcgaatttgtctacggcgactataaagtctatgatgtccgcaaaatgattgcgaaaagcgaacaagaaattggcaaagcgacagcgaaatactttttttacagcaacatcatgaacttttttaaaacggaaatcacactggcgaacggcgaaattagaaaaagaccgcttattgaaacgaacggtgaaacaggcgaaattgtttgggataaaggcagagattttgcaacagttagaaaagttctgagcatgccgcaagtcaacatcgtgaaaaaaacagaagttcagacaggcggatttagcaaagaatcaattcttccgaaacgcaactcagacaaactgattgcgcgtaaaaaagactgggacccgaaaaaatacggtggctttgattcaccgacagttgcatattcagttctggttgttgcgaaagtggaaaaaggcaaatccaaaaaacttaaaagcgtgaaagaacttctgggcatcacaattatggaacgctcgagctttgaaaaaaacccgatcgactttctggaagccaaaggctataaagaagtgaaaaaagaccttattatcaaactgccgaaatacagcctgtttgaactggaaaatggcagaaaacgcatgctggcatcagcaggcgaacttcagaaaggcaatgaactggcactgccgtcaaaatatgttaactttctgtatctggcgagccattacgaaaaacttaaaggctcaccggaagataacgaacagaaacaactgtttgtcgaacagcataaacattacctggacgaaatcatcgaacaaatcagcgaattttcaaaacgcgttattctggcagatgcgaacctggataaagttcttagcgcatataacaaacaccgggataaaccgattagagaacaagcggaaaatatcattcacctgtttacactgacaaatcttggcgcaccggcagcgtttaaatactttgatacaacaattgaccgcaaacgctacacaagcacaaaagaagttctggacgcaacactgattcatcaatcaattacaggcctttatgaaacgagcattgatctgtcacaactgggaggcgattgatgctgtccagactgtccgctgtgtaaaaaaaaggaataaaggggggttgacattattttactgatatgtataatataatttgtataagaaaatgaatggtacttgttaaaaattgttttagagctagaaatagcaagttaaaataaggctagtccgttatcaacttgaaaaagtggcaccgagtcggtgctttgcggccgcagatctgggaccaataataatgactagagaagaaagaatgaagattgttcatgaaattaaggaacgaatattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactggctagcatggcaagacccagaaaagttctgggagatcccgctttgcataagcgtattatagtggatgacgcgggctttgttgtttacacttcttgcacctgctgacggcaatcatccctatctatgaaatcgagatttcagcaggccgttattttcgagagagttaaatctatattcattgtttttattttggtaaggacataccggattttaggtttggattaccggtcgagttagcttgtcttttcgcccactaccgtgtcgatgcgggagcaatttaccagaagcacttaccgattgatagttttttattccggtgattgcaaagtttcataaactctgagaattcaataggggtaataccccgctttgaggggcgcggcattttatgcgccccgagtatttattcttaaaatttttaaattaatgtatctatataaaaaggagatgctttcggtgtactgccaaagcatctccacaaaagatagtgcatatctgca

SEQ ID NO: 8 - DNA, 3629 bp, synthetic construct
  misc_feature 1..3629: note = amyE:GFP-gDNA(p4199).dna locus
  source 1..3629: mol_type = other DNA
aatgtgttacacctgttttgtaattctcaaggaaaacatgaggcgcttttgcaatatcatcaggataaagcacagctacagacctggcattgatcgtgcctgtcagtttaccatcgttcacttgaaatgaacccgctccagctttattgtcatacctgccatcaggcaattttgttgccgtattgatagagacagaggatgaacctgcatttgccagcacaacgccatgtgagccgcgctgattcataaatatctggttgtttccattcgggttcgagagttcctcaggctgtccagccatcacattgtgaaatctattgaccgcagtgatagcctgatcttcaaataaagcactcccgcgatcgcctatttggcttttccccgggaacctcacaccatttccgcctccctcaggtctggaaaagaaaagaggcgtactgcctgaacgagaagctatcaccgcccagcctaaacggatatcatcatcgctcatccatgtctgtagacaaattgtgaaaggatgtacttaaacgctaacggtcagctttattgaacagtaatttaagtatatgtccaatctagggtaagtaaattgagtatcaatataaactttatatgaacataatcaacgaggtgaaatcatgagcaatttgattaacggaaaaataccaaatcaagcgattcaaacattaaaaatcgtaaaagatttatttggaagttcaatagttggagtatatctatttggttcagcagtaaatggtggtttacgcatttacagcgatgtagatgttctagtcgtcgtgaatcatagtttacctcaattaactcgaaaaaaactaacagaaagactaatgactatatcaggaaagattggaaatacggattctgttagaccacttgaagttacggttataaataggagtgaagttgtcccttggcaatatcctccaaaaagagaatttatatacggtgagtggctcaggtgtggatttgagaatggacaaattcaggaaccaagctatgatcctgatttggctattgttttagcacaagcaagaaagaatagtatttctctatttggtcctgattcttcaagtatacttgtctccgtacctttgacagatattcgaagagcaattaaggattctttgccagaactaattgaggggataaaaggtgatgagcgtaatgtaattttaaccctagctcgaatgtggcaaacagtgactactggtgaaattacctcgaaagatgtcgctgcggaatgggctatacctcttttacctaaagagcatgtaactttactggatatagccagaaaaggctatcggggagagtgtgatgataagtgggaaggactatattcaaaggtgaaagcactcgttaagtatatgaaaaattctatagaaacttctctcaattaggctaattttattgcaataacaggtgcttacttttaaaactactgatttattgataaatattgaacaatttttgggaagaataaagcgtcctcttgtgaaattagagaacgctttattactttaatttagtgaaacaatttgtaactattgaaaatagaaagaaattgttccttcgatagtttattaatattagtggagctcagtgagagcgaagcgaacacttgattttttaattttctatcttttataggtcattagagtatacttatttgtcctataaactatttagcagcataatagatttattgaataggtcatttaagttgagcatattaggggaggaaaatcttggagaaatatttgaagaacccgagatctagatcaggtaccgcaacgttcgcagatgctgctgaagagattattaaaaagctgaaagcaaaaggctatcaattggtaactgtatctcagcttgaagaagtgaagaagcagagaggctattgaataaatgagtagaaagcgccatatcggcgcttttcttttggaagaaaatatagggaaaatggtacttgttaaaaattcggaatatttatacaatatcatatgtatcacattgaaaggaggagggaagctttatgagtaaaggagaagaacttttcactggagttgtcccaattcttgttgaattagatggcgatgttaatgggcaaaaattctctgttagtggagagggtgaaggtgatgcaacatacggaaaacttacccttaaatttatttgcactactgggaagctacctgttccatggccaacgcttgtcactactctcacttatggtgttcaatgcttttctagatacccagatcatatgaaacagcatgactttttcaagagtgccatgcccgaaggttatgtacaggaaagaactatattttacaaagatgacgggaactacaagacacgtgctgaagtcaagtttgaaggtgatacccttgttaatagaatcgagttaaaaggtattgattttaaagaagatggaaacattcttggacacaaaatggaatacaattataactcacataatgtatacatcatggcagacaaaccaaagaatggcatcaaagttaacttcaaaattagacacaacattaaagatggaagcgttcaattagcagaccattatcaacaaaatactccaattggcgatggccctgtccttttaccagacaaccattacctgtcccaccaatctgccctttccaaagatcccaacgaaaagagagatcacatgatccttcttgagtttgtaacagctgctgggattacacatggcatggatgaactatacaaataatgctgtccagactgtccgctgtgtaaaaaaaaggaataaaggggggttgacattattttactgatatgtataatataatttgtataagaaaatgaatggtacttgttaaaaattgttttagagctagaaatagcaagttaaaataaggctagtccgttatcaacttgaaaaagtggcaccgagtcggtgctttgcggccgcgtcgattgacactaaagggatccagaagcggcaacacgctaatcaataaaaaaacgctgtgcggttaaagggcacagcgttttttgtgtatgaatcgaaaaagagaacagatcgcaggtctcaaaaatcgagcgtaaagggctgatccgcggccgcgcgtcaacaatgacctttatgccatattcttcagcggctgcacacatttctttaaattcttgttcagtacctaagtaacggttgccaatttgatacgatgtcggctgatacagccagtaccagttcgacatgcttttatctccttgattcccttcctttacttggttaatcggagatgtctgaatggctgtatatcctgcatcatgaatatccttcatattgtgttttaacgtattgaacgaccaattccatgcatgaagaatggttccgcttttgatcgacggtgctgtaagctcattcgatttgttcgccgtttcagcactcgcagccgccggtcctgccagaaccaaatgaaacagcaataaaaatccagcgaataacggcagtaaagaggttttgaatcgttttgcaaacattcttgacactccttatttgattttttgaagacttacttcggagtcaaaaatccctcttacttcattcttccgcttcctccttt

SEQ ID NO: 9 - DNA, 6881 bp, synthetic construct
  misc_feature 1..6881: note = pel:cas9d-gDNA(GFP) locus
  source 1..6881: mol_type = other DNA
taggccaccagacgttgggatccaatgatggctggggcgcgtactcgaccggcacgacaggcggatcaaaagcatcctcctcaaatgtgtataccgtcagcaacagaaaccagcttgtctcggcattagggaaggaaacgaacacaacgccaaaaatcatttatatcaagggaacgattgacatgaacgtggatgacaatctgaagccgcttggcctaaatgactataaagatccggagtatgatttggacaaatatttgaaagcctatgatcctagcacatggggcaaaaaagagccgtcgggaacacaagaagaagcgagagcacgctctcagaaaaaccaaaaagcacgggtcatggtggatatccctgcaaacacgacgatcgtcggttcagggactaacgctaaagtcgtgggaggaaacttccaaatcaagagtgataacgtcattattcgcaacattgaattccaggatgcctatgactattttccgcaatggttgtaaaacgacggccagtgaattctgatcaaatggttcagtgagagcgaagcgaacacttgattttttaattttctatcttttataggtcattagagtatacttatttgtcctataaactatttagcagcataatagatttattgaataggtcatttaagttgagcatattagaggaggaaaatcttggagaaatatttgaagaacccgaggatctagatcaggtaccgcaacgttcgcagatgctgctgaagagattattaaaaagctgaaagcaaaaggctatcaattggtaactgtatctcagcttgaagaagtgaagaagcagagaggctattgaataaatgagtagaaagcgccatatcggcgcttttcttttggaagaaaatatagggaaaatggtacttgttaaaaattcgaaatatttatacaatatcatatgtatcacattgaaaggggaggagaatcatggacaaaaaatacagcatcggcctggctattggcacaaattcagttggctgggcagttatcacagacgaatataaagttccgagcaaaaaatttaaagtcctgggcaatacagatcgccatagcatcaaaaaaaacctgattggcgcactgctgtttgattcaggcgaaacagcagaagcaacaagacttaaaagaacagcaagacgcagatatacaagacgcaaaaatcgcatttgctatctgcaagaaatctttagcaacgaaatggcgaaagtcgacgacagcttttttcatagactggaagaatcatttctggtcgaagaagataaaaaacacgaacgccatccgatttttggcaacattgttgatgaagtcgcgtatcatgaaaaatacccgacaatttatcatctgcgcaaaaaactggttgacagcacagataaagcagatcttcgcctgatttatctggcactggcacatatgatcaaatttagaggccattttctgatcgaaggcgatctgaatccggataattcagatgtcgacaaactgtttattcagctggtccagacatataaccagctgtttgaagaaaatccgattaatgcatcaggcgttgatgcaaaagcaattctgtcagcaagactgtcaaaatcaagacgcctggaaaatctgattgcacaactgcctggcgaaaaaaaaaatggactgtttggcaatcttattgcactgtcactgggcctgacaccgaactttaaatcaaattttgatctggcggaagatgcgaaactgcaactttcaaaagatacgtatgatgacgatctggataatctgctggcgcaaattggcgatcaatatgcagatctttttctggcagcgaaaaatctgtcagatgcaattctgctgtcagatattctgcgcgtcaatacagaaattacaaaagcaccgctgagcgcgagcatgattaaaagatatgatgaacatcatcaggacctgacactgctgaaagcactggttagacaacaactgccggaaaaatacaaagaaatcttttttgatcagagcaaaaacggctatgcaggctatattgatggcggagcatcacaagaagaattttacaaatttatcaaaccgatcctggaaaaaatggatggaacagaagaactgctggttaaactgaatcgcgaagatttactgagaaaacagcgcacatttgataatggctcaattccgcatcaaattcatctgggcgaactgcatgcgattcttagacgccaagaagatttttatccgtttctgaaagacaaccgggaaaaaattgaaaaaatcctgacatttcgcatcccgtattatgtcggaccgctggcaagaggcaattcaagatttgcatggatgacacgcaaaagcgaagaaacaattacaccgtggaattttgaagaagtcgttgataaaggcgcaagcgcacaatcatttattgaacgcatgacgaactttgacaaaaacctgccgaatgaaaaagtcctgccgaaacattcactgctgtatgaatactttacggtctataatgaactgacgaaagtcaaatatgtcacagaaggcatgagaaaaccggcatttctgtcaggcgaacagaaaaaagcgattgtcgatcttctgtttaaaacgaaccgcaaagtcacagtgaaacagctgaaagaagattactttaaaaaaatcgaatgctttgatagcgtcgaaatctcaggcgtcgaagatagatttaatgcaagcctgggcacatatcatgatctgctgaaaatcatcaaagataaagattttctggataacgaagaaaacgaagatatcctggaagatattgtgctgacactgacgctttttgaagatcgcgaaatgattgaagaacgcctgaaaacatatgcgcacctgtttgatgataaagtcatgaaacaacttaaacgcagacgctatacaggctggggcagactttcaagaaaactgattaacggcattcgcgataaacaaagcggcaaaacaatcctggattttctgaaatcagatggctttgcgaatcgcaattttatgcagctgattcatgatgacagcctgacgtttaaagaagatattcagaaagcacaagtttcaggccaaggcgattcactgcatgaacatattgcaaatctggcaggctcaccggcaatcaaaaaaggcattctgcaaacagttaaagtcgtcgatgaactggttaaagttatgggcagacataaaccggaaaacatcgttattgaaatggcacgcgaaaatcagacaacacaaaaaggacagaaaaattcacgcgaacggatgaaaagaattgaagaaggcattaaagaactgggcagccaaatcctgaaagaacatccggttgaaaatacacagctgcagaacgaaaaactgtatctgtattatctgcagaatggacgcgatatgtatgtcgatcaagaactggatattaatcgcctgagcgattatgatgtggatgctattgttccgcagagctttcttaaagatgatagcatcgataacaaagtcctgacacgctcagataaaaacagaggcaaatcagataatgtcccgtcagaagaggttgtcaaaaaaatgaaaaactactggcgtcaactgctgaacgcgaaacttattacacaacgcaaatttgacaatctgacaaaagcagaaagaggcggactgtcagaacttgataaagcgggttttatcaaaagacagctggtcgaaacacgccagattacaaaacatgttgcgcaaattctggatagccgcatgaacacaaaatatgacgaaaacgataaactgatccgggaagtcaaagtcattacgctgaaatcaaaactggtcagcgattttcgcaaagactttcagttttacaaagtccgcgaaatcaacaactaccatcatgcacatgatgcatatctgaatgcagttgtcggcacagcgcttatcaaaaaataccctaaactggaaagcgaatttgtctacggcgactataaagtctatgatgtccgcaaaatgattgcgaaaagcgaacaagaaattggcaaagcgacagcgaaatactttttttacagcaacatcatgaacttttttaaaacggaaatcacactggcgaacggcgaaattagaaaaagaccgcttattgaaacgaacggtgaaacaggcgaaattgtttgggataaaggcagagattttgcaacagttagaaaagttctgagcatgccgcaagtcaacatcgtgaaaaaaacagaagttcagacaggcggatttagcaaagaatcaattcttccgaaacgcaactcagacaaactgattgcgcgtaaaaaagactgggacccgaaaaaatacggtggctttgattcaccgacagttgcatattcagttctggttgttgcgaaagtggaaaaaggcaaatccaaaaaacttaaaagcgtgaaagaacttctgggcatcacaattatggaacgctcgagctttgaaaaaaacccgatcgactttctggaagccaaaggctataaagaagtgaaaaaagaccttattatcaaactgccgaaatacagcctgtttgaactggaaaatggcagaaaacgcatgctggcatcagcaggcgaacttcagaaaggcaatgaactggcactgccgtcaaaatatgttaactttctgtatctggcgagccattacgaaaaacttaaaggctcaccggaagataacgaacagaaacaactgtttgtcgaacagcataaacattacctggacgaaatcatcgaacaaatcagcgaattttcaaaacgcgttattctggcagatgcgaacctggataaagttcttagcgcatataacaaacaccgggataaaccgattagagaacaagcggaaaatatcattcacctgtttacactgacaaatcttggcgcaccggcagcgtttaaatactttgatacaacaattgaccgcaaacgctacacaagcacaaaagaagttctggacgcaacactgattcatcaatcaattacaggcctttatgaaacgagcattgatctgtcacaactgggaggcgattgatgctgtccagactgtccgctgtgtaaaaaaaaggaataaaggggggttgacattattttactgatatgtataatataatttgtataagaaaatgtctgttagtggagagggtgagttttagagctagaaatagcaagttaaaataaggctagtccgttatcaacttgaaaaagtggcaccgagtcggtgctttgcggccgcagatctgggaccaataataatgactagagaagaaagaatgaagattgttcatgaaattaaggaacgaatattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaacgatacattaataggtacgaaaaagcaactttttttgcgcttaaaaccagtcataccaataacttaagggtaactagcctcgccggaaagagcgaaaatgcctcacatttgtgccacctaaaaaggagcgatttacatatgagttatgcagtttgtagaatgcaaaaagtgaaatcagctggactaaaaggggccgcagagtagaatggaaaaggggatcggaaaacaagtatataggaggagacctatttatggcttcagaaaaagacgcaggaaaacagtcagcagtaaagcttgttccattgcttattactgtcgctgtgggactaatcatctggtttattcccgctccgtccggacttgaacctaaagcttggcatttgtttgcgatttttgtcgcaacaattatcggctttatctccaagcccttgccaatgggtgcaattgcaatttttgcattggcggttactgcactaactggaacactatcaattgaggatacattaagcggattcgggaataagaccatttggcttatcgttatcgcattctttatttcccggggatttatcaaaaccggtctcggtgcgagaatttcgtatgtattcgttcagaaattcggaaaaaaaacccttggactttcttattcactgctattcagtgatttaatactttcacctgctattccaagtaatacggc